(19) United States
(12) Patent Application Publication
(10) Pub. No.: US 2022/0249561 A1
(43) Pub. Date: Aug. 11, 2022

(54) CHIMERIC ANTIGEN RECEPTOR-EXPRESSING CELLS TARGETING ALK

(71) Applicant: SHINSHU UNIVERSITY, Matsumoto-shi, Nagano (JP)

(72) Inventors: Yozo Nakazawa, Matsumoto-shi (JP); Shoji Saito, Matsumoto-shi (JP); Shigeki Yagyu, Kyoto-shi (JP); Shigeru Nakano, Azumino-shi (JP); Takaki Momose, Azumino-shi (JP); Kenta Hitomi, Azumino-shi (JP)

(73) Assignee: SHINSHU UNIVERSITY, Matsumoto-shi, Nagano (JP)

(21) Appl. No.: 17/614,766

(22) PCT Filed: May 29, 2020
(86) PCT No.: PCT/JP2020/021348

§ 371 Date: Apr. 19, 2022

(30) Foreign Application Priority Data
May 31, 2019 (JP) 2019-103074

(51) Int. Cl. A61K 35/17; C12N 15/62; C12N 15/85; C12N 5/0783; C07K 14/725; A61P 35/00

(52) U.S. Cl. CPC A61K 35/17; C12N 15/625; C12N 15/85; C12N 5/0636; C07K 14/7051; A61K 38/00; C12N 2510/00; C12N 2800/107; C12N 2501/2307; C12N 2501/2315; A61P 35/00

(57) ABSTRACT

The present invention is intended to develop a chimeric antigen receptor (CAR) that is effective against solid tumor expressing anaplastic lymphoma kinase (ALK). The present invention provides a polynucleotide encoding a CAR protein comprising a target binding domain binding to an extracellular ligand binding region of ALK, a transmembrane domain, and an intracellular signaling domain. The target binding domain of the polynucleotide is selected from among FAM150A, FAM150B, and fragments thereof binding to the extracellular ligand binding region of ALK. The present invention also provides a genetically modified cell comprising the polynucleotide introduced thereinto.

### TECHNICAL FIELD

The present invention relates to a genetically modified cell expressing a chimeric antigen receptor, which is useful in the field of adoptive immunotherapy, and a method for producing the same.

### BACKGROUND ART

Adoptive immunotherapy using T cells expressing a chimeric antigen receptor (CAR) (CAR-T) targeting a tumor-related antigen has been reported to have a potent antitumor effect, and its development has advanced rapidly in recent years. Particularly, the development of CARs aimed at the treatment of B cell tumor has advanced and already reached clinical application. In the field of solid tumors, however, the development of CARs is still in progress and clinical application thereof is not yet realized.

The correlation between anaplastic lymphoma kinase (ALK), which is a receptor tyrosine kinase expressed at a high level in tumor tissue of neuroblastoma, and solid tumor has been reported for a long time (Non-Patent Literature 1). In large-scale cohort studies conducted in Japan, it has been reported that, in addition to abnormality of ALK gene, high ALK scores (quantification of pathological ALK expression) would affect the prognosis of neuroblastoma (Non-Patent Literature 2). Thus, development of a novel therapeutic agent targeting ALK is expected for the treatment of solid tumors including neuroblastoma.

In the past, ALK inhibitory agents using small molecule compounds had been developed, and therapeutic agents for ALK fusion-positive lung cancer have been applied in clinical settings (Non-Patent Literature 3). However, ALK that is expressed at a high level in the case of neuroblastoma is not of a gene fusion type, but is reported to be a wild-type or point mutation type having an extracellular ligand-binding domain, and small molecule inhibitory agents are considered ineffective. Accordingly, development of novel therapeutic agents has been desired.

As CAR-T therapeutic techniques targeting ALK, research on scFv-type CAR-T using as an antigen binding domain, a single-chain antibody fragment (scFv) derived from an antibody against ALK has been reported (Patent Literature 1, Non-Patent Literature 4).

Meanwhile, ALK is a receptor tyrosine kinase (RTKs) as with the leukocyte tyrosine kinase (LTK), and it was known as an orphan receptor whose physiological ligand has not yet been found (Non-Patent Literature 5). In recent years, however, low-molecular-weight proteins, FAM150A (11.5 kDa) and FAM150B (14.5 kDa), were found to functionally bind to ALK and LTK (Non-Patent Literatures 6 and 7). Further, a fragment of the FAM150B referred to as an “AD domain (N terminal defect)” is reported to exert the binding ability and the phosphorylation ability equivalent to those exerted by the full-length sequence on ALK (Non-Patent Literature 8).

### CITATION LIST

### Patent Literatures

### Patent Literature 1: WO 2015/069922

### Non-Patent Literatures

Non-Patent Literature 1: Nat. Rev. Cancer, 2013; 13: 685-700
Non-Patent Literature 2: Oncotarget, 2017, vol. 8, No. 64, pp. 107513-107529
Non-Patent Literature 3: Cold Spring Harb. Mol. Case Stud., 2017: 3: a001115
Non-Patent Literature 4: Molecular Therapy, 2017, vol. 25, 9, 2189-2201
Non-Patent Literature 5: PNAS, 2014, vol. 111, 44, 15741-15745
Non-Patent Literature 6: eLife, 2015, 4, e09811
Non-Patent Literature 7: PNAS, 2015, vol. 112, 52, 15862-15867
Non-Patent Literature 8: PNAS, 2018, vol. 115, 33, 8340-8345

### SUMMARY OF INVENTION

### Technical Problem

Antitumor activity of scFv-type CAR-T is reported to be limited both in vitro and in vivo. A problem as a CAR-T therapeutic agent is also reported: antitumor activity of scFv-type CAR-T depends on the ALK expression level in a target tumor, and activity thereof on a tumor expressing low levels of ALK cannot be expected. Under the above circumstances, further development of CARs that can be effective on ALK-expressing solid tumors is desired. Solutions

The present inventors have conducted concentrated studies in consideration of applicability of ligand-type CAR using, as an antigen binding domain, FAM150A and FAM150B, which may be physiological ligands of ALK (hereinafter, referred to as “ALK.CAR”), as an adoptive immunotherapeutic agent against solid tumors. In addition, they have focused on regions highly homologous in FAM150A and FAM150B, and examined ALK.CAR using truncated fragments lacking the N terminus and/or the C terminus as an antigen binding domain (hereinafter, referred to as “TrALK.CAR”).

As a result, they discovered that T cells into which ALK.CAR and TrALK.CAR had been introduced (hereinafter, referred to as “ALK.CAR-T” and “TrALK.CAR-T,” respectively) exerts potent antitumor activity against ALK-expressing tumor cells, which has led to the completion of the present invention.

Specifically, the present invention provides the followings:
[1] A polynucleotide encoding a chimeric antigen receptor (CAR) protein comprising a target binding domain that binds to an extracellular ligand binding region of anaplastic lymphoma kinase (ALK), a transmembrane domain, and an intracellular signaling domain, wherein the target binding domain is selected from among FAM150A, FAM150B, and fragments thereof binding to the extracellular ligand binding region of ALK.
[2] The polynucleotide according to [1], wherein the target binding domain is a truncated fragment of FAM150A and/or FAM150B.
[3] The polynucleotide according to [1], wherein the target binding domain is a polypeptide consisting of an amino acid sequence selected from the group consisting of SEQ ID NO: 154 (FAM150A), SEQ ID NO: 146 (TrFAM150A), SEQ ID NO: 155 (FAM150B), and SEQ ID NO: 148 (TrFAM150B).
[4] The polynucleotide according to [1], wherein the target binding domain is a polypeptide consisting of an amino acid sequence having 90% or higher sequence identity to the amino acid sequence represented by SEQ ID NO: 146 (TrFAM150A) or SEQ ID NO: 148 (TrFAM150B).
[5] A vector comprising the polynucleotide according to any of [1] to [4].
[6] A genetically modified cell comprising the polynucleotide according to any of [1] to [4] or the vector according to [5] introduced thereinto.
[7] The cell according to [6], which expresses a CAR protein binding to an ALK-expressing cell on a cell membrane.
[8] A method for preparing a CAR protein-expressing cell comprising introducing the polynucleotide according to any of [1] to [4] or the vector according to [5] into a cell.
[9] The method according to [8], wherein the polynucleotide or the vector is introduced into the cell by the transposon method.
[10] The method according to [9], wherein the transposon method is the piggyBac method.
[11] A kit comprising the vector according to [5] used for preparing a CAR protein-expressing cell targeting an ALK-expressing cell.
[12] A therapeutic agent for a disease associated with an ALK-expressing cell, comprising the cell according to [6] or [7].
[13] A pharmaceutical composition comprising the therapeutic agent according to [12] and a pharmaceutically acceptable carrier.
[14] The therapeutic agent according to [12] or the composition according to [13], wherein the disease associated with an ALK-expressing cell is a solid tumor selected from among neuroblastoma, breast cancer, uterine cancer, endometrial cancer, ovarian cancer, melanoma, astroglioma, Ewing's sarcoma, glioblastoma, retinoblastoma, rhabdomyoblastoma, non-small cell lung cancer, prostate cancer, and urothelial cancer.
[15] A method for treatment of a solid tumor selected from among neuroblastoma, breast cancer, uterine cancer, endometrial cancer, ovarian cancer, melanoma, astroglioma, Ewing's sarcoma, glioblastoma, retinoblastoma, rhabdomyoblastoma, non-small cell lung cancer, prostate cancer, and urothelial cancer, comprising administering a therapeutically effective amount of the therapeutic agent according to [12] or the composition according to [13] to a patient.

The present description encompasses the contents disclosed in Japanese Patent Application No. 2019-103074 on which the priority of the present application is based.

### Advantageous Effects of Invention

The present invention provides a genetically modified cell that binds to a target cell expressing ALK on a cell surface and exerts antitumor effects. Thus, the cell according to the present invention can be used as an adoptive immunotherapeutic agent for solid tumor diseases including neuroblastoma.

### BRIEF DESCRIPTION OF DRAWINGS

FIG. 1 shows a vector map of CAR001.

FIG. 2 shows a vector map of CAR 002 (FAM150A-28z).

FIG. 3 shows a vector map of CAR 003 (FAM150B-28z).

FIG. 4 shows a vector map of CAR 004 (hALK48-28z).

FIG. 5 shows a vector map of CAR 005 (ALK48-28z).

FIG. 6 shows antitumor activity of CAR-T 002 to CAR-T 005 or mock-T cells (Effector, E) obtained from Donor-1 against neuroblastoma cell line SH-SY5Y (Target, T). The tumor cell proliferation rates (%) are calculated relative to tumor cell proliferation rate (100%) in the group to which no CAR-T was administered (i.e., the CAR-T non-administered group: No CAR). A: E:T=4:1; B: E:T=2:1; C: E:T=1:1. The effects of CAR-T 002 to CAR-T 005 to kill SH-SY5Y were confirmed, and the effects of CAR-T 002 and CAR-T 003 were higher than those of CAR-T 004 and CAR-T 005.

FIG. 7 shows antitumor activity of CAR-T 002 to CAR-T 006 or mock-T cells (E) obtained from Donor-2 against neuroblastoma cell line SH-SY5Y (T). The tumor cell proliferation rates (%) are calculated relative to tumor cell proliferation rate (100%) in the CAR-T non-administered group (No CAR). A: E:T=4:1: B: E:T=2:1: C: E:T=1:1. The effects of CAR-T 002 to CAR-T 005 to kill SH-SY5Y were confirmed, and the effects of CAR-T 002 and CAR-T 003 were higher than those of CAR-T 004 and CAR-T 005.

FIG. 8 shows antitumor activity of CAR-T 002 to CAR-T 005 or mock-T cells (E) obtained from Donor-1 against neuroblastoma cell line NB-1 (T). A: E:T=4:1; B: E:T=2:1; C: E:T=1:1. The effects of CAR-T 002 to CAR-T 005 to kill NB-1 were confirmed, and the effects of CAR-T 002 and CAR-T 003 were higher than those of CAR-T 004 and CAR-T 005.

FIG. 9 shows antitumor activity of CAR-T 002 to CAR-T 006 or mock-T cells (E) obtained from Donor-2 against neuroblastoma cell line NB-1 T). A: E:T=4:1: B: E:T=2:1; C: E:T=1:1). The effects of CAR-T 002 to CAR-T 005 to kill NB-1 were confirmed, and the effects of CAR-T 002 and CAR-T 003 were higher than those of CAR-T 004 and CAR-T 005.

FIG. 10 shows antitumor activity of CAR-T 002 to CAR-T 006 or mock-T cells (E) obtained from Donor-2 against neuroblastoma cell line IMR-32 (T). A: E:T=4:1; B: E:T=2:1; C: E:T=1:1. The effects of CAR-T 002 to CAR-T 005 to kill IMR-32 were confirmed, and the effects of CAR-T 003 were particularly high.

FIG. 11 shows a domain map of the protein used in Example 4.

FIG. 12 shows the results of verification of binding of ALK 001P and LTK 001P to Ligand 001P and Ligand 002P via surface plasmon resonance analysis. Ligand 001P shows a higher binding ability to LTK 001P (B) than to ALK 001P (A). Ligand 002P is slowly detached from ALK 001P (C) and LTK 001P (D), which shows a high binding ability thereto.

FIG. 13 shows a vector map of CAR 007 (FAM150ATr-28z).

FIG. 14 shows a vector map of CAR 008 (FAM150BTr-28z).

FIG. 15 shows antitumor activity of CAR-T 003, CAR-T 008, and CAR-T 006 or mock-T cells (E) obtained from Donor-1 against SH-SY5Y (1). A: E:T=4:1; B: E:T=2:1; C: E:T=1:1. The effects of CAR-T 003 and CAR-T 008 to kill SH-SY5Y were confirmed. In particular, the effects of CAR-T 008 to kill SH-SY5Y were higher than those of CAR-T 003, and also high at E:T=1:1.

FIG. 16 shows antitumor activity of CAR-T 003, CAR-T 008, and CAR-T 006 or mock-T cells (E) obtained from Donor-1 against IMR-32 (T). A: E:T=4:1; B: E:T=2:1: C: E:T=1:1. The effects of CAR-T 003 and CAR-T 008 to kill IMR-32 were confirmed. In particular, the effects of CAR-T 008 to kill IMR-32 were higher than those of CAR-T 003, and also high at E:T=1:1.

FIG. 17 shows antitumor activity of CAR-T 003, CAR-T 008, and CAR-T 006 or mock-T cells (E) obtained from Donor-2 against SH-SY5Y (T) (A: E:T=4:1; B: E:T=2:1; C: E:T=1:1), and the proliferation curves plotting the relative tumor cell counts (D: E:T=4:1; E: E:T=2:1; F: E:T=1:1). The effects of CAR-T 003 and CAR-T 008 to kill SH-SY5Y were confirmed. In particular, the effects of CAR-T 008 to kill SH-SY5Y were higher than those of CAR-T 003, and also high at E:T=1:1.

FIG. 18 shows antitumor activity of CAR-T 003, CAR-T 008, and CAR-T 006 or mock-T cells (E) obtained from Donor-2 against IMR-32 (T) (A: E:T=4:1; B: E:T=2:1; C: E:T=1:1), and the tumor cell proliferation curves (D: E:T=4:1; E: E:T=2:1; F: E:T=1:1). The effects of CAR-T 003 and CAR-T 008 to kill IMR-32 were confirmed. In particular, the effects of CAR-T 008 were higher than those of CAR-T 003, and also high at E:T=1:1.

FIG. 19 shows a domain map of the protein used in Examples 9 and 10.

FIG. 20-1 shows the results of verification of binding of ALK 001P and LTK 001P to Ligand 005P and Ligand 006P via surface plasmon resonance analysis.

FIG. 20-2 shows the results of verification of binding of ALK 001P and LTK 001P to Ligand 003P and Ligand 007P via surface plasmon resonance analysis.

FIG. 20-3 shows the results of verification of binding of ALK 001P and LTK 001P to Ligand 008P and Ligand 009P via surface plasmon resonance analysis.

FIG. 20-4 shows the results of verification of binding of ALK 001P and LTK 001P to Ligand 010P and Ligand 011P via surface plasmon resonance analysis.

FIG. 21-1 shows the results of verification of binding of ALK 001P and LTK 001P to Ligand 012P and Ligand 013P via surface plasmon resonance analysis.

FIG. 21-2 shows the results of verification of binding of ALK 001P and LTK 001P to Ligand 014P and Ligand 015P via surface plasmon resonance analysis.

FIG. 21-3 shows the results of verification of binding of ALK 001P and LTK 001P to Ligand 016P and Ligand 017P via surface plasmon resonance analysis.

FIG. 21-4 shows the results of verification of binding of ALK 001P and LTK 001P to Ligand 018P and Ligand 019P via surface plasmon resonance analysis.

FIG. 22-1 shows the results of verification of binding of ALK 001P and LTK 001P to Ligand 020P and Ligand 021P via surface plasmon resonance analysis.

FIG. 22-2 shows the results of verification of binding of ALK 001P and LTK 001P to Ligand 022P and Ligand 023P via surface plasmon resonance analysis.

FIG. 22-3 shows the results of verification of binding of ALK 001P and LTK 001P to Ligand 024P via surface plasmon resonance analysis.

FIG. 23 shows a vector map of CAR 009 (FAM150A-8αBBz).

FIG. 24 shows a vector map of CAR 010 (FAM150B-8αBBz).

FIG. 25 shows a vector map of CAR 011 (FAM150ATr-8αBBz).

FIG. 26 shows a vector map of CAR 012 (FAM150BTr-8αBBz).

FIG. 27 shows a vector map of CAR 013 (hALK48-8αBBz).

FIG. 28 shows a vector map of CAR 014 (ALK48-8αBBz).

FIG. 29 shows antitumor activity of CAR-T 009 to CAR-T 014 or mock-T cells (E) obtained from Donor-1 against SH-SY5Y (T) (A: E:T=4:1; B: E:T=2:1; C: E:T=1:1), and the tumor cell proliferation curves (D: E:T=4:1: E: E:T=2:1; F: E:T=1:1).

FIG. 30 shows antitumor activity of CAR-T 009 to CAR-T 014 or mock-T cells (E) obtained from Donor-1 against MDA-MB231 ffLuc (T) (A: E:T=4:1; B:E:T=2:1; C:E:T=1:1), and the tumor cell proliferation curves (D: E:T=4:1; E: E:T=2:1; F: E:T=1:1).

FIG. 31 shows a vector map of CAR 015 (FAM150BT14-28z).

FIG. 32 shows a vector map of CAR 016 (FAM150BT15-28z).

FIG. 33 shows a vector map of CAR 017 (FAM150BT17-28z).

FIG. 34 shows a vector map of CAR 018 (FAM150BT18-28z).

FIG. 35 shows a vector map of CAR 019 (FAM150BT19-28z).

FIG. 36 shows antitumor activity of CAR-T 015, CAR-T 016, CAR-T 017, CAR-T 018, CAR-T 019, CAR-T 008, and CAR-T 006 or mock-T cells (E) obtained from Donor-1 against SH-SY5Y (T), NB-1 (T), IMR32 (T), and MDA-MB231 ffLuc(T) (E:T=1:1).

FIG. 37 shows a vector map of CAR 020 (FAM50BTr-BBz dCH2CH3).

FIG. 38 shows antitumor activity of CAR-T 006, CAR-T 012, and CAR-T 020 or mock-T cells (E) obtained from Donor-1 against SH-SY5Y (T), NB-1 (T), IMR32 (T), and MDA-MB231 ffLuc (T) (E:T=1:1).

FIG. 39 shows a vector map of pEHX-ALK.

FIG. 40 shows a vector map of pEHX-LTK.

FIG. 41 shows the results of flow cytometric analysis of ALK-expressing clones and LTK-expressing clones.

FIG. 42 shows a vector map of CAR 021 (FAM150BTr-28z dCH2CH3).

FIG. 43 shows a vector map of CAR 022 (ALK48 scFv-28z dCH2CH3).

FIG. 44 shows transitions in the cell index over time when CAR-T 021, CAR-T 022, or mock-T cells are added to A24 cells highly expressing ALK (A: E:T=40:1; B: E:T=20:1; C: E:T=10:1). “A24” indicates the results when cultured in the absence of effector cells.

FIG. 45 shows transitions in the cell index over time when CAR-T 021, CAR-T 022, or mock-T cells are added to L10 cells highly expressing LTK (A: E:T=40:1: B: E:T=20:1; C: E:T=10:1). “L10” indicates the results when cultured in the absence of effector cells.

### EMBODIMENTS OF THE INVENTION

The embodiments of the present invention are described in detail below.

As described above, the present invention provides a polynucleotide encoding a chimeric antigen receptor (CAR) protein comprising a target binding domain that binds to an extracellular ligand binding region of anaplastic lymphoma kinase (ALK), a transmembrane domain, and an intracellular signaling domain, wherein the target binding domain is selected from among FAM150A. FAM150B, and fragments thereof binding to the extracellular ligand binding region of ALK.

In the case where a single antigen is targeted in immunotherapy, it is preferable that CAR be capable of recognizing a plurality of antigens, from the viewpoint of, for example, reduction of a risk of cancer recurrence caused by the growth of an antigen quenching escape mutant that can be generated during the process of various therapies. Accordingly, an aspect of the present invention provides a bispecific CAR. For example, an embodiment of the present invention provides a polynucleotide encoding a CAR protein having a target binding domain that binds to extracellular ligand binding regions of ALK and LTK.

The term “anaplastic lymphoma kinase (ALK)” used herein refers to a receptor tyrosine kinase that belongs to the insulin receptor superfamily identified in 1994 as a fusion molecule with nucleophosmin in case of anaplastic large cell lymphoma. ALK is a cell membrane-binding protein that is known to be expressed on the cell surface of solid tumor such as neuroblastoma, breast cancer, or hung cancer. The extracellular domain of ALK functions as a receptor including a region that is rich in MAM and glycine, and the intracellular kinase domain is associated with signal transduction after a ligand binds to the receptor. When a ligand binds to the receptor and ALK is then activated, tumor cells are proliferated by the action of the kinase.

Examples of ALK-expressing tumors include solid tumors, such as neuroblastoma, breast cancer, uterine cancer, endometrial cancer, ovarian cancer, melanoma, astroglioma, Ewing's sarcoma, glioblastoma, retinoblastoma, rhabdomyoblastoma, non-small cell lung cancer, prostate cancer, and urothelial cancer. High-level expression of ALK in such tumor cells is reported. In addition, canceration caused by mutation in the kinase domain is known. It is also known that, when ALK forms a fusion protein with another gene in a cell, ALK is constantly activated to develop a tumor. There is no extracellular ligand-binding region in a fusion protein, and a fusion protein is not included in the target in the present invention, Accordingly, the term “ALK-expressing cell” used herein refers to, in particular, a cell that expresses ALK having an extracellular ligand-binding region.

The amino acid sequence and the nucleotide sequence of ALK protein are described in, for example, the NCBI database under Accession NOs. NM_004304.4 and NM_004304.5.

Leukocyte tyrosine kinase (LTK) is a receptor tyrosine kinase, a partial structure thereof was identified in 1988 and a full-length thereof was reported to be a 100 kDa glycosylated protein in 1991. LTK is known to be expressed in B-lymphocyte precursors, B lymphocytes, hematopoietic stem cells, the brain, the placenta, and various cancer cells. The amino acid sequence and the nucleotide sequence of LTK protein are described in, for example, the NCBI database under Accession NO. NM_002344.5.

The “chimeric antigen receptor (CAR)” used herein refers to a modified receptor that can impart its target specificity to cells such as T cells (e.g., naive T cells, stem cell memory T cells, central memory T cells, effector memory T cells, or a combination thereof). CAR is also known as an artificial T cell receptor, a chimeric T cell receptor, or a chimeric immunoreceptor.

The CAR for use in the method of the present invention has a target binding domain that binds to an extracellular ligand binding region of ALK, a transmembrane domain, and an intracellular signaling domain. The term “domain” used herein refers to a region within a polypeptide and folded into a particular structure independently of other regions.

The term “polynucleotide” used herein encompasses, but is not limited to, natural or synthetic DNA and RNA, for example, genomic DNA, cDNA (complementary DNA), mRNA (messenger RNA), rRNA (ribosomal RNA), shRNA (small hairpin RNA), snRNA (small nuclear RNA), snoRNA (small nucleolar RNA), miRNA (microRNA), and/or tRNA.

The term “encoding” used herein means that a predetermined nucleotide sequence has a code for information on the amino acid sequence of a predetermined protein or (poly)peptide, as usually used in the art. In the present description, both sense and antisense strands are used in the context of “encoding.”

The CAR protein according to the present invention comprises a “target binding domain” that binds to an extracellular ligand binding region of ALK. The target binding domain is capable of binding to the extracellular ligand binding region of ALK, and it enables immune responses to the target cell expressing ALK on its cell surface.

As the target binding domain accordingly, ligands for ALK; i.e., FAM150A, FAM150B, and fragments thereof binding to an extracellular ligand binding region of ALK, can be used. In a preferable embodiment of fragments, truncated fragments of FAM150A and/or FAM150B can be used.

The sequence information of FAM150A can be obtained as NCBI Accession Number: NM %_207413.4, and FAM150A can be prepared on the basis of the nucleotide sequence represented by SEQ ID NO: 1, which encodes the translation region thereof. The sequence information of FAM150B can be obtained as NCBI Accession Number: NM_001002919.2, and FAM150B can be prepared on the basis of the nucleotide sequence represented by SEQ ID NO: 3, which encodes the translation region thereof.

In the present invention, full-length FAM150A and FAM150B existing in nature can be used as target domains. Specific examples of target binding domains that can be used include FAM150A represented by SEQ ID NO: 145 (Uniprot No: Q6UXT8-1(1-129)) or SEQ ID NO: 154 (Uniprot No: Q6UXT8-1(28-129)) and FAM150B represented by SEQ ID NO: 147 (Uniprot No: Q6UX46-1(1-152)) or SEQ ID NO: 155 (Uniprot No: Q6UX46-1(25-152)). Truncated fragments thereof can also be used as ALK-binding fragments. Specifically, in the amino acid sequence represented by SEQ ID NO: 147 (FAM150B), a polypeptide consisting of an amino acid sequence of, for example, amino acids 67 to 152, amino acids 69 to 152, amino acids 71 to 152, amino acids 73 to 152, amino acids 75 to 152, amino acids 77 to 152, amino acids 79 to 152, amino acids 81 to 152, amino acids 83 to 152, amino acids 85 to 152, amino acids 87 to 152, amino acids 89 to 152, amino acids 91 to 152, amino acids 93 to 152, amino acids 71 to 150, or amino acids 71 to 148, can be used as a target binding domain.

More preferably, a polypeptide consisting of an amino acid sequence selected from the group consisting of SEQ ID NO: 154 (FAM150A), SEQ ID NO: 146 (TrFAM150A), SEQ ID NO: 155 (FAM150B), and SEQ ID NO: 148 (TrFAM150B) can be used as a target binding domain.

A polypeptide consisting of an amino acid sequence having 90% or higher sequence identity to the amino acid sequence represented by SEQ ID NO: 146 (TrFAM150A) or SEQ ID NO: 148 (TrFAM150B) can also be used.

The ability of the target binding domain to bind to an extracellular ligand binding region of ALK is, for example, 100 nM or lower, preferably 10 nM or lower, and more preferably 5 nM or lower, in terms of a KD value. The binding ability may be lower than the antigen-antibody binding ability.

The CAR protein can optionally comprise an “extracellular spacer domain” between the target binding domain located extracellularly and the transmembrane domain. The extracellular spacer domain is desirably a sequence that promotes the binding of CAR to the antigen and facilitates signal transduction into a cell. For example, an Fc fragment of an antibody or a fragment or a derivative thereof, a hinge region of an antibody or a fragment or a derivative thereof, a CH2 region of an antibody, a CH3 region of an antibody, an artificial spacer sequence, or a combination thereof can be used.

In an aspect of the present invention, (i) hinge, CH2, and CH3 regions of IgG4, (ii) a hinge region of IgG4, (iii) hinge and CH2 regions of IgG4, (iv) a hinge region of CD8a. (v) hinge, CH2 and CH3 regions of IgG1, (vi) a hinge region of IgG1, or (vii) hinge and CH2 regions of IgG1, or a combination thereof can be used as the extracellular spacer domain. For example, a region having the following amino acid sequence (SEQ ID NO: 149) can be suitably used as the hinge region of IgG1, although the extracellular spacer domain is not limited thereto.

UniProt No.: P01857 (99-110)

| EPKSCDKTHTCP | PCDPA | EPKSPDKTHTCP |
|---|---|---|
| Hinge | Spacer | Hinge |

A region having the amino acid sequence represented by SEQ ID NO: 150 and a region having the amino acid sequence represented by SEQ ID NO: 151 can be suitably used as the CH2 region and the CH3 region of IgG1, respectively.

In a preferred aspect, hinge, CH2, and CH3 regions of human IgG1 or a part thereof can be used as the extracellular spacer domain.

In a preferred aspect, (i) a hinge region of human IgG1 (SEQ ID NO: 149) by itself, (ii) a hinge region (SEQ ID NO: 149), a CH2 region (SEQ ID NO: 150), and a CH3 region (SEQ ID NO: 151) of human IgG1 in combination, (iii) a hinge region (SEQ ID NO: 149) and a CH3 region (SEQ ID NO: 151) of human IgG1 in combination, or (iv) a CH3 region (SEQ ID NO: 151) by itself can be used as the extracellular spacer domain.

In an aspect of the present invention, a spacer sequence represented by the formula (G4S)n can be used as the artificial spacer sequence for use in the extracellular spacer domain. In the formula, n is 1 to 10, and n is preferably 3. A spacer having such a spacer sequence may be referred to as a “peptide linker.” Peptide linkers suitably used in the art can adequately be used in the present invention. In this case, the configuration and the chain length of the peptide linker can be adequately selected without impairing the function of the resulting CAR protein.

The extracellular spacer domain is not particularly limited. The extracellular spacer domain can be adequately selected from those listed above or further modified in accordance with the general technical knowledge in the art and used in the present invention.

Nucleotide sequences encoding the respective amino acid sequences of domains can be ligated, inserted into a vector, and expressed in host cells, so that the extracellular spacer domain can be located between the target binding domain and the transmembrane domain. Alternatively, the extracellular spacer domain may be modified using a polynucleotide encoding a plasmid CAR protein produced in advance as a template.

The modification of the extracellular spacer domain is useful when, for example, improvement in the CAR gene expression rate in host cells harboring an introduced polynucleotide encoding CAR, signal transduction, aging of cells, distribution in tumor, antigen recognition, or influence on in vivo activity, is taken into consideration.

The CAR protein comprises an extracellular domain comprising a target binding domain and, optionally, an extracellular spacer domain, a transmembrane domain, and an intracellular domain comprising an intracellular signaling domain and, optionally, a co-stimulatory domain. As is well known in the art, the “transmembrane domain” is a domain having affinity to a lipid bilayer constituting cell membrane, whereas both the extracellular domain and the intracellular domain are hydrophilic domains.

The transmembrane domain is not particularly limited, as long as the CAR protein can be present on the cell membrane without impairing the functions of the target binding domain and the intracellular signaling domain. A polypeptide derived from the same protein as that of the co-stimulatory domain described below may function as the transmembrane domain. For example, a transmembrane domain such as CD28, CD3ε, CD8a, CD3, CD4, or 4-1BB can be used. For example, human CD28 (Uniprot No.: P10747 (153-179)) can be used as the transmembrane domain. Specifically, a domain having an amino acid sequence encoded by the nucleotide sequence: NCBI Accession Number: NM_006139.3 (679-759) can be suitably used as the transmembrane domain.

The CAR protein can optionally comprise a “co-stimulatory domain.” The co-stimulatory domain specifically binds to a costimulatory ligand, thereby mediating cellular costimulatory responses, such as CAR-T cell proliferation, cytokine production, functional differentiation, and target cell death, although the cellular costimulatory responses are not limited thereto. For example, CD27, CD28, 4-1BB(CD137), CD134 (OX40), Dap10, CD27, CD2, CD5, CD30. CD40, PD-1, ICAM-1, LFA-1 (CD11a/CD18), TNFR-I, TNFR-II, Fas, or Lck can be used as the co-stimulatory domain. For example, human CD28 (Uniprot No.: P10747 (180-220)) or 4-1BB (GenBank: U03397.1) can be used as the co-stimulatory domain. Specifically, a domain having an amino acid sequence encoded by the nucleotide sequence represented by NCBI Accession Number: NM_006139.3 (760-882) can be suitably used as the co-stimulatory domain.

When the transmembrane domain and the co-stimulatory domain both derived from human CD28 are used, for example, a domain having the amino acid sequence represented by SEQ ID NO: 152 can be used.

The CAR protein comprises an “intracellular signaling domain.” The intracellular signaling domain transmits signals required for exerting effector functions of immune cells. For example, a human CD3′ chain, FcγRIII, FcεRI, a cytoplasmic end of an Fc receptor, a cytoplasmic receptor having an immunoreceptor tyrosine activation motif (ITAM), or a combination thereof can be used as the intracellular signaling domain. For example, a human CD3C chain (e.g., nucleotides 299-637 of NCBI Accession Number: NM_000734.3) can be used as the intracellular signaling domain. Specifically, a domain having the amino acid sequence represented by SEQ ID NO: 153 can be suitably used as the intracellular signaling domain.

In order to accelerate CAR protein secretion, a signal(or leader) sequence that induces protein transfer during or after translation is adequately added to the N terminus of the protein. Examples of useful signal sequences that can be suitably used in the present invention include, but are not limited to, human immunoglobulin (Ig) heavy chain signal peptide, CD8a signal peptide, and human GM-CSF receptor a signal peptide. Ig heavy chain signal peptides derived from, for example, IgG1, IgG2, IgG3, IgA1, and IgM can be suitably used.

The polynucleotide of interest can be easily produced according to a conventional technique. Nucleotide sequences encoding the respective amino acid sequences of domains can be obtained from NCBI RefSeq IDs or GenBank Accession numbers indicating the amino acid sequences, and the polynucleotide of the present invention can be produced in accordance with standard molecular biological and/or chemical procedures. For example, nucleic acids can be synthesized on the basis of these nucleotide sequences. Also, DNA fragments obtained by polymerase chain reaction (PCR) from a cDNA library can be combined to produce the polynucleotide of the present invention.

Thus, the polynucleotide encoding the CAR protein can be produced by ligating the respective polynucleotides encoding the domains described above. Genetically modified cells can be produced by introducing this polynucleotide to adequate cells. Alternatively, the CAR protein may be produced by using, as a template, a polynucleotide encoding an existing CAR protein having the same constituents except for the target binding domain, and recombining the target binding domains in accordance with a conventional technique.

According to need, one or more domains, such as the extracellular spacer domain, can be modified by inverse-PCR (iPCR) or other means, using, as a template, a polynucleotide encoding an existing CAR protein. The technique of modifying the extracellular spacer domain is described in, for example, Oncoimmunology, 2016, Vol. 5, No. 12, e1253656.

Any general method of polynucleotide introduction may be employed to prepare genetically modified cells without particular limitation. When a polynucleotide is introduced using a vector, for example, a lentivirus vector, a retrovirus vector, a foamy vims vector, or an adeno-associated virus vector can be used, although a vector is not limited thereto. Alternatively, polynucleotide introduction can be carried out by a non-viral method based on a transposon method. The transposon method can be performed with the use of a plasmid transposon, and a sleeping beauty transposon system(described in, for example, Huang X, Guo H, et al., Mol. Ther., 2008; 16: 580-9: Singh H. Manui P R, et al., Cancer Res., 2008; 68: 2961-71: Deniger D C, Yu J, et al., PLoS One, 2015; 10: e0128151: Singh H, Moyes J S, et al., Cancer Gene Ther., 2015: 22: 95-100: Hou X, Du Y, et al., Cancer Biol. Ther., 2015; 16: 8-16; Singh H, Huls H, et al., Immunnol. Rev., 2014; 257: 181-90; and Maiti S N, Huls H. et al., J. Immunother., 2013: 36: 112-23) or a piggyBac transposon system (described in, for example, Nakazawa Y, Huye L E, et al., J. Imunnother., 2009; 32: 826-36; Galvan D L. Nakazawa Y, et al., J Immumother., 2009:32: 837-44:NakazawaY, Huye L E, et al., Mol. Ther., 2011:19: 2133-43;Huye L E, Nakazawa Y. et al., Mol. Ther., 2011; 19: 2239-48; Saha S. Nakazawa Y, et al., J. Vis. Exp., 2012: (69): e4235: Nakazawa Y. Saha S, et al., J. Immunother., 2013: 36: 3-10; Saito S, Nakazawa Y. et al., Cytotherapy, 2014; 16: 1257-69; and Nakazawa et al., Journal of Hematology & Oncology, 2016, 9:27) can be preferably used.

In the case where using the piggyBac transposon system, typically, a plasmid carrying a gene encoding piggyBac transposase (referred to as a piggyBac plasmid herein) and a plasmid having a structure where the polynucleotide encoding a CAR protein is flanked by piggyBac inverted repeat sequences are introduced (transfection). Transfection can be carried out by various approaches, such as electroporation, nucleofection, lipofection, or the calcium phosphate method. Both the plasmids can contain a poly A addition signal sequence, a reporter gene, a selection marker gene, an enhancer sequence, and the like.

Examples of apparatuses that can be used for electroporation include, but are not limited to, 4D-Nucleofector (Lonza Japan Ltd.), NEPA21 (Nepa Gene Co., Ltd.), and Maxcyte G T (Maxcyte, Inc.), and such apparatuses can be operated according to their respective instruction manuals.

According to the method described above, gene introduction into 1×10⁶ to 2×10⁷ cells can be performed.

As cells into which the polynucleotides are to be introduced, cells derived from mammals, such as humans. T cells or a cell population containing T cells derived from non-human mammals, such as monkeys, mice, rats, pigs, cattle, and dogs can be used. Cells releasing a cytotoxic protein (perforin, granzyme, etc.) are preferably used. Specifically, for example, a cell population containing T cells, precursor cells of T cells (hematopoietic stem cells, lymphocyte precursor cells, etc.), and/or NK-T cells can be used. Further examples are cells capable of differentiating into these cells, including various stem cells such as ES cells and iPS cells. The T cells encompass CD8-positive T cells, CD4-positive T cells, regulatory T cells, cytotoxic T cells, and tumor-infiltrating lymphocytes. The cell population containing T cells and precursor cells of T cells includes PBMCs. The cells described above may be collected from a living organism, the expansion culture products thereof, or the established cell lines thereof. When transplanting the CAR-expressing cells produced or cells differentiated therefrom into a living organism, it is desirable to introduce a nucleic acid into cells collected from the living organism itself or a living organism of the same species thereas.

As T cells for use in adoptive immunotherapy into which the polynucleotide is to be introduced to produce the genetically modified cells of the present invention, T cells expected to have sustainable antitumor effects, such as stem cell memory T cells, can be used. The stem cell memory T cells can be analyzed in accordance with a conventional technique and easily confirmed as described in, for example. Yang Xu, et al., Blood, 2014; 123: 3750-3759.

In one embodiment, for example, CD45R0-, CD62L+, CD45RA+, and CCR7+ T cells can be used as stem cell memory T cells.

The present invention also provides a vector comprising the polynucleotide of the present invention.

The present invention also provides a genetically modified cell comprising the polynucleotide of the present invention or the vector of the present invention introduced thereinto. The genetically modified cell of the present invention can express a CAR protein binding to an ALK-expressing cell on a cell membrane.

The present invention further provides a method for preparing a CAR protein-expressing cell comprising introducing the polynucleotide of the present invention or the vector of the present invention into a cell.

CAR protein-expressing cells can be cultured and proliferated by any means without particular limitation. For example, a polynucleotide encoding a CAR protein is introduced into a cell in the manner described above, and non-specific or CAR-specific stimuli can then be applied to the cell, so as to activate the CAR protein-expressing cell. A method of cell stimulation is not limited. As a method of non-specific cell stimulation, for example, stimuli can be applied using anti-CD3 antibody and/or anti-CD28 antibody. As a method of CAR-specific stimulation, for example, stimuli can be applied using artificial antigen presenting cells (aAPCs) comprising CAR-binding antigen molecules or costimulatory factors expressed in K562 or other tumor cell lines. While culture conditions are not particularly limited, for example, culture is suitably carried out at 37° C. for 1 to 21 days.

In the method according to the present invention described above, a method of introducing a polynucleotide into a cell is not limited, and the transposon method is suitably employed. As the transposon method, the transposon system described above or other systems suitable in the present invention may be employed. The method according to the present invention can be adequately performed by the piggyBac method, although the method is not limited thereto.

In another aspect of non-specific stimulation, after a cell population comprising T cells is stimulated by one or more types of virus peptide antigens, cells in which the virus growth ability is inactivated by a conventional technique are used as feeder cells to promote activation of cells into which CAR has been introduced. As virus peptide antigens, for example, an AdV antigen peptide mixture, a CMV antigen peptide mixture, an EBV antigen peptide mixture, or a combination thereof can be used. Specific examples are shown in the examples below.

In order to enhance cell viability and proliferation rate, culture can be carried out in the presence of one or more types of cytokines. For example, culture can be preferably carried out in the presence of cytokines, such as IL-7 and IL-15.

The present invention further provides a kit used for preparing a CAR protein-expressing cell that targets an ALK-expressing cell comprising the vector according to the present invention. The kit according to the present invention can adequately contain, for example, a reagent, a buffer, a reaction vessel, and instructions that are necessary for preparing a CAR protein-expressing cell. The kit according to the present invention can be suitably used for preparing the genetically modified cell according to the present invention.

The cell according to the present invention induces receptor-specific immune responses to a target cell expressing ALK on its surface. Thus, signal transduction takes place in the cell and the cell is then activated. Activation of a CAR-expressing cell can be confirmed using, as an indicator, release of cytokines, an enhanced cell proliferation rate, and changes in cell surface molecules, for example, although the indicator varies depending on the type of host cells or the intracellular domain of CARs. Release of cytotoxic proteins, such as perforin and granzyme, would damage cells expressing receptors.

The genetically modified cell according to the present invention can be used as a therapeutic agent for a disease associated with an ALK-expressing cell. Diseases expected to be cured by the therapeutic agent of the present invention are not limited, provided that they have sensitivity to the cell. For example, diseases are associated with a cell expressing ALK on its cell membrane, and include solid tumors, such as neuroblastoma, breast cancer, uterine cancer, endometrial cancer, ovarian cancer, melanoma, astroglioma, Ewing's sarcoma, glioblastoma, retinoblastoma, rhabdomyoblastoma, non-small cell lung cancer, prostate cancer, and urothelial cancer.

The therapeutic agent according to an aspect of the present invention is an anti-cancer agent against an ALK-expressing tumor cell, such as the solid tumor described above. While the therapeutic agent or anti-cancer agent according to the present invention can be used alone, it can also be used in combination with an agent and/or treatment of different mechanisms.

Thus, the therapeutic agent according to the present invention can be in the form of a pharmaceutical composition comprising the therateutic agent alone or in combination with other active ingredients. The therapeutic agent or the pharmaceutical composition according to the present invention can be applied by topical or systemic administration. While the route of administration is not limited, for example, intravenous administration is preferable in the case of treatment of neuroblastoma. The pharmaceutical composition may comprise, in addition to the therapeutic agent according to the present invention and other active ingredients, a carrier, an excipient, a buffer, a stabilizer, and other substances that are commonly used in the art, depending on the route of administration. A dose of the therapeutic agent according to the present invention varies depending on, for example, body weight, age, and severity of disease of the patient. While a dose is not particularly limited, for example, 10⁴ to 10¹⁰ CAR-positive cells can be administered per kg body weight 1 to several times a day, every 2 days, every 3 days, every week, every 2 weeks, every month, every 2 months, or every 3 months.

The present invention further provides a method of treatment of a solid tumor selected from among neuroblastoma, breast cancer, uterine cancer, endometrial cancer, ovarian cancer, melanoma, astroglioma, Ewing's sarcoma, glioblastoma, retinoblastoma, rhabdomyoblastoma, non-small cell lung cancer, prostate cancer, and urothelial cancer, comprising administering a therapeutically effective amount of the therapeutic agent or pharmaceutical composition according to the present invention to a patient. The therapeutically effective amount and the administration regimen can be adequately determined in consideration of various factors as described above.

### EXAMPLES

Hereafter, the present invention is described in greater detail with reference to the examples, although the present invention is not limited to the following examples.

### Example 1

Preparation of FAM150A (CAR 002; FAM150A-28z), FAM150B (CAR 003; FAM150B-28z), humanized ALK48 scFv (CAR 004; hALK48-28z), or mouse ALK48 scFv-type (CAR 005: ALK48-28z) CAR-expressing plasmids Artificial synthesis of FAM150A, FAM150B, humanized ALK48 scFv, or mouse ALK48 scFv gene

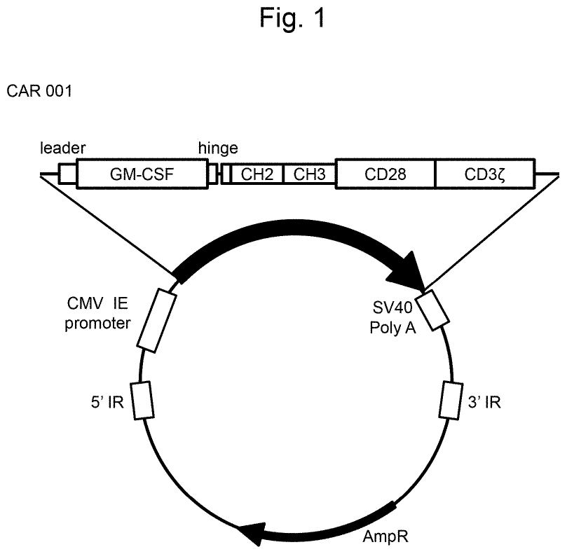

In order to prepare a CAR-expressing plasmid of the structure similar to that of the GMR CAR-expressing plasmid described in WO 2018/052142 (CAR001, a vector map thereof is shown in FIG. 1), FAM150A, FAM150B, humanized ALK48 scFv, and mouse ALK48 scFv genes to be incorporated into the plasmid were synthesized.

A DNA sequence (XhoI-leader-FAM150A-Hinge-DraIII; SEQ ID NO: 2) comprising a restriction enzyme XhoI cleavage sequence and a leader sequence added to upstream a translation region (206 to 592 bp; SEQ ID NO: 1) of FAM150A (NCBI Accession Number: NM_207413.3) and a hinge region and a restriction enzyme DraIII cleavage sequence added to downstream thereof was designed. The leader sequence was a DNA sequence to be translated into the amino acid sequence of a human immunoglobulin (Ig) heavy chain signal peptide(SEQ ID NO: 143).

A DNA sequence (XhoI-leader-FAM150B-Hinge-DraIII; SEQ ID NO: 4) comprising a restriction enzyme cleavage sequence, a leader sequence, and a hinge region added to a translation region (354 to 809 bp; SEQ ID NO: 3) of FAM150B (NCBI Accession Number: NM_001002919.2) was also designed. The leader sequence was a DNA sequence to be translated into the amino acid sequence of a human immunoglobulin (Ig) heavy chain signal peptide (SEQ ID NO: 143).

The designed sequences were artificially synthesized as DNA sequences codon-optimized for human (synthesis was commissioned to Eurofins Genomics K.K.).

Separately, on the basis of the amino acid sequences of humanized ALK48 scFv and mouse ALK48 scFv (SEQ ID NO: 5 and SEQ ID NO: 6) described in WO 2015/069922, DNA sequences (XhoI-leader-humanized ALK48 scFv-Hinge-DraIII (SEQ ID NO: 7) and XhoI-leader-mouse ALK48 scFv-Hinge-DraIII (SEQ ID NO: 8)) comprising the restriction enzyme cleavage sequence, the leader sequence, and the hinge region added to the DNA sequence codon-optimized for human were designed as with the case of FAM150A and FAM150B, artificially synthesized, and then incorporated into the pEX-K4J1 vector (synthesis was commissioned to Eurofins Genomics K.K.).

Preparation of FAM150A-type CAR-expressing plasmid (CAR 002), FAM150B-type CAR-expressing plasmid (CAR 003), humanized ALK48 scFv-type CAR-expressing plasmid (CAR 004), and mouse ALK48 scFv-type CAR-expressing plasmid (CAR 005)

CAR 001 (about 1 μg equivalent: prepared by the method described in WO 2015/069922) was digested with restriction enzymes XhoI and DraIII (New England Biolabs) at 37° C. for about 2 hours. The 4 types of genes artificially synthesized above (about 1 μg equivalent each) were also digested with restriction enzymes XhoI and DraIII at 37° C. for about 2 hours.

After the enzyme treatment, the reaction solution was separated via 1% or 2% agarose gel electrophoresis, the enzyme-treated CAR 001 fragment (on the vector side) and the artificially synthesized gene-inserted fragment (SEQ ID NO: 2, 4, 7 or 8) cleaved from the pEX-K4J1 vectors were removed from the gel, and the fragments were purified using NucleoSpin Gel and PCR Clean-up® (MACHEREY-NAGEL. Takara Bio Inc.). The purified vector fragment was ligated to the purified insert fragment using the DNA ligation kit (Mighty Mix, Takara Bio Inc.). E. coli DH5a (Toyobo Co. Ltd.) cells were transformed using the ligated cyclic plasmid and then cultured on an LB agar medium containing 100 μg/ml carbenicillin for about 16 hours.

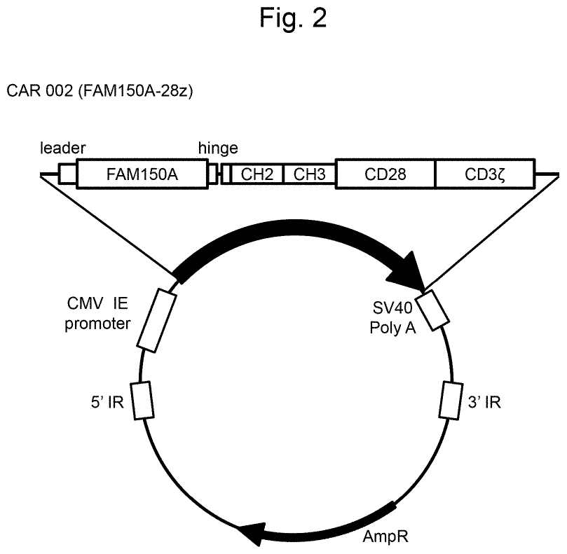

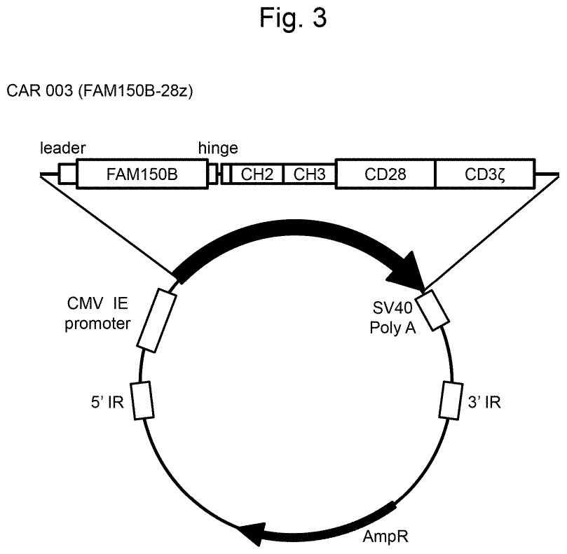

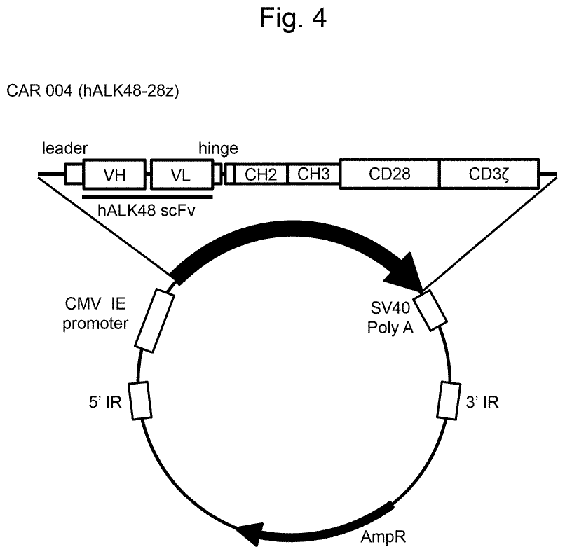

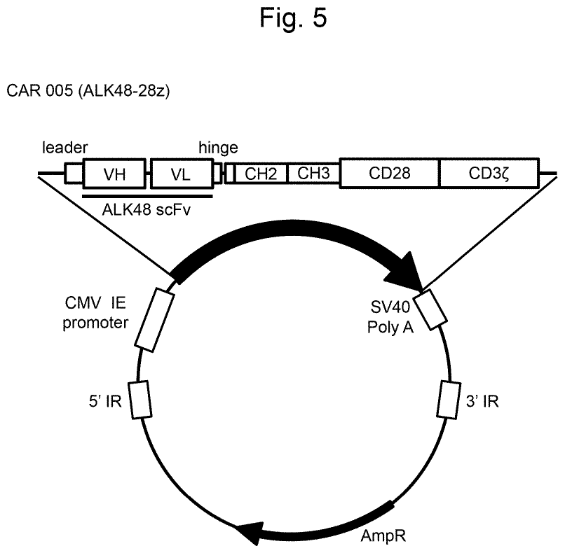

The appeared colonies were further cultured in an LB liquid medium containing 100 sg/ml carbenicillin for about 16 hours. Plasmids were purified from the cultured E. coli cells using QIAprep Spin Miniprep Kit (Qiagen), nucleotide sequences were determined, and plasmids in which target nucleotide sequence insertion was observed were obtained as FAM150A-type CAR-expressing plasmid (CAR 002; FAM150A-28z), FAM150B-type CAR-expressing plasmid (CAR 003; FAM150B-28z), humanized ALK48 scFv-type CAR-expressing plasmid (CAR 004; hALK48-28z), or mouse ALK48 scFv-type CAR-expressing plasmid (CAR 005: ALK48-28z). Nucleotide sequence analysis was commissioned to Eurofins Genomics K.K. Vector maps of the prepared plasmids are shown in FIGS. 2 to 5.

### Example 2

### Culture and Proliferation of CAR-T Cells

Peripheral blood mononuclear cells (PBMCs) were separated by any of the methods described below.

### Day 0: Separation of Peripheral Blood Mononuclear Cells (PBMCs) (Density Gradient Centrifugation)

Peripheral blood samples were obtained from healthy adult donors and diluted to 2-fold with D-PBS (FUJIFILM Wako Pure Chemical Corporation). The diluted peripheral blood was superposed on Ficoll-Paque PLUS (GE Healthcare), and centrifugation was carried out at 400× g for 30 minutes to fractionate the PBMC layer. The fractionated PBMCs were washed 2 times with D-PBS and isolated via centrifugation.

### Day 0: Separation of PBMCs (Separation Using SepMate-50)

Peripheral blood samples were obtained from healthy adult donors and diluted to 2-fold with D-PBS (FUJIFILM Wako Pure Chemical Corporation). SepMate-50 (STEMCELL Technologies) was filled with 15 ml of Ficoll-Paque PLUS in advance, the diluted peripheral blood samples were superposed thereon, centrifugation was carried out at 1,200×g for 10 minutes, and the supernatant containing PBMCs and plasma was transferred to another 50-ml centrifuge tube via decantation. After D-PBS was added to the supernatant to adjust the amount to 50 ml, centrifugation was carried out at 300×g for 8 minutes, the supernatant was removed, pellets were washed with D-PBS, centrifugation was carried out again at 300× g for 8 minutes, and the supernatant was removed to isolate PBMCs.

### Day 0: Virus Peptide Pulsing of PBMCs

The isolated PBMCs were stimulated with virus peptides using PepTivator Peptide Pools® (Miltenyi Biotec). Specifically, PBMCs were suspended in Peptide Pools each comprising AdV5 Hexon, CMV pp65, EBV BZLF1, and EBV EBNA-1 added at 0.05 μg/μl to 50 μl of D-PBS, and the resulting suspensions were stimulated with virus peptides at 37° C. for 30 minutes. Thereafter, 5 ml of D-PBS was added to PBMCs to prepare a suspension, and the suspension was then irradiated with UV for 4 minutes. PBMCs irradiated with UV were collected, the number of cells was counted, the cells were suspended at 0.5 to 4×10⁶ cells/2 ml/well in the TexMACS medium containing 10 ng/ml IL-7 and 5 ng/ml IL-15, and the cell suspension was transferred as feeder cells to a 24-well treated culture plate.

### Day 0: Gene Introduction

CAR-expressing plasmids were introduced into 15×10⁶ PBMCs. Specifically, 5 μg of any of CAR 002 to CAR 005 obtained in Example 1, 5 μg of pCMV-piggyBac plasmid, and 100 μl of the P3 Primary Cell solution included in P3 Primary Cell 4D-Nucleofector™X Kit (Lonza Japan Ltd.) were mixed with each other, and 15×10⁶ PBMCs were suspended in the mixture. All the suspended cells were transferred to Nucleocuvette, and the genes were introduced into cells through electrical pulses by the 4D-Nucleofection system (Program No: FI-115). The cells into which the genes had been introduced through electrical pulses were allowed to stand at room temperature for 10 minutes, all the cells were added to a 24-well treated culture plate containing feeder cells, and culture was initiated.

As the mock-T cells without gene introduction, 15×10⁶PBMCs were cultured in the same manner. As CAR-T cells not damaging solid tumors, cells subjected to the same gene introduction procedure using CD19 scFv-type CAR-expressing plasmid (CAR 006; CD19-28z, prepared in the same manner as described in WO 2018/052142) were prepared. A half of the medium was discarded during the culture and a half of TexMACS medium containing 20 ng/ml IL-7 and 10 ng/ml IL-15 was added to exchange media, approximately every other day.

Day 7: Stimulation of CAR-T Cells with Antibody and Proliferation Thereof

A 24-well non-treated culture plate was coated with D-PBS containing anti-CD3 antibody and anti-CD28 antibody at 37° C. for 2 hours to prepare an antibody-coated plate, the total amount of the cell suspension was seeded on the plate, and the CAR-T cells cultured above were stimulated with antibodies for 2 days. On Day 9, the total amount of the cell suspension was transferred to a G-Rex 6-well plate (Wilson Wolf) filled with 30 ml of the TexMACS medium containing 10 ng/ml IL-7 and 5 ng/ml IL-15, and the cells were further cultured for 7 days and proliferated up to Day 16. The CAR expression rates in the ALK CAR-T cells proliferated up to Day 16 into which either of CAR 002 to CAR 005 had been introduced and the CD19 CAR-T cells into which CAR 006 had been introduced were determined in the manner described below. The experiment may be performed one day before or after the designated day.

PBMCs obtained from 2 healthy adult donors were subjected to the procedure described above.

### Day 16: Evaluation of CAR Expression Rate

The number of CAR-T cells into which the CAR-expressing plasmids had been introduced were counted, and 1 to 2×10⁵ cells were subjected to flow cytometric analysis to evaluate the CAR expression rates in the ALK CAR-T cells and in the CD19 CAR-T cells.

1 to 2×10⁵ cells were sampled, suspended with 2 μl of FITC Goat Anti-Human IgG (H+L) antibody (Jackson ImmunoResearch Inc) and 5 μl of APC Anti-Human CD3 antibody (Miltenyi Biotec), and the resulting suspension was subjected to antibody labeling at 4C under shading conditions for 20 minutes. Thereafter, the cells were washed with 500 μl of D-PBS, and precipitated via centrifugation. After the supernatant was removed, the cells were re-suspended in 500 μl of D-PBS. The resulting sample was analyzed using FACSCalibur (BD Biosciences), and the expression rates of IgG1/CD3-positive ALK CAR (CAR 002 to CAR 005) or CD19 CAR (CAR 006) were determined.

The CAR-T cells obtained by gene introduction through electrical pulses into PBMCs using expression plasmids (CAR 002 to CAR 006) and T cell culture and proliferation are described herein as follows.
CAR-T 002: FAM150A CD28-type CAR-T (FAM150A-28z CAR-T)
CAR-T 003: FAM150B CD28-type CAR-T (FAM150B-28z CAR-T)
CAR-T 004: Humanized ALK48 scFv CD28-type CAR-T (hALK48-28z CAR-T)
CAR-T 005: Mouse ALK48 scFv CD28-type CAR-T (ALK48-28z CAR-T)
CAR-T 006: CD19 scFv CD28-type CAR-T (CD19-28z CAR-T)

The amino acid sequence of CAR 002 and the nucleotide sequence encoding the same are represented by SEQ ID NO: 13 and SEQ ID NO: 9, respectively.

The amino acid sequence of CAR 003 and the nucleotide sequence encoding the same are represented by SEQ ID NO: 14 and SEQ ID NO: 10, respectively.

The amino acid sequence of CAR 004 and the nucleotide sequence encoding the same are represented by SEQ ID NO: 15 and SEQ ID NO: 11, respectively.

The amino acid sequence of CAR 005 and the nucleotide sequence encoding the same are represented by SEQ ID NO: 16 and SEQ ID NO: 12, respectively.

Table 1 shows CAR expression rates in CAR-T cells obtained from PBMCs derived from 2 donors.

TABLE 1

| Donor | Plasmid | CAR-T | Expression rate |
|---|---|---|---|
| Donor-1 | CAR 002 | CAR-T 002 | 22.3 |
| | CAR 003 | CAR-T 003 | 15.7 |
| | CAR 004 | CAR-T 004 | 22.1 |
| | CAR 005 | CAR-T 005 | 23.3 |
| Donor-2 | CAR 002 | CAR-T 002 | 33.2 |
| | CAR 003 | CAR-T 003 | 41.2 |
| | CAR 004 | CAR-T 004 | 36.9 |
| | CAR 005 | CAR-T 005 | 56.2 |
| | CAR 006 | CAR-T 006 | 60.7 |

### Example 3

Comparison of Antitumor Activity of FAM150A CD28-Type CAR-T, FAM150B CD28-Type CAR-T, Humanized ALK48 scFv CD28-Type CAR-T, and Mouse ALK48 scFv CD28-Type CAR-T
Measurement of antitumor activity of CAR-T

In order to measure antitumor activity of CAR-T 002 to CAR-T 005 obtained in Example 2, co-culture with solid tumor cells was conducted. In this example, neuroblastoma cell line SH-SY5Y cells (DS Pharma Biomedical Co., Ltd.), NB-1 cells (JCRB Cell Bank), or IMR-32 cells (DS Pharma Biomedical Co., Ltd.) were used as target tumor cells.

SH-SY5Y cells, NB-1 cells, and IMR32 cells were subjected to passage culture and used for co-culture test. In passage culture, D-MEM/Ham's F-12 medium containing 15% FBS, 1% penicillin/streptomycin, and 1% non-essential amino acid solution is used for SH-SY5Y cells. RPMI 1640 medium containing 10% FBS and 1% penicillin/streptomycin is used for NB-1 cells, and E-MEM medium containing 10% FBS, 1% penicillin/streptomycin, and 1% non-essential amino acid solution is used for IMR-32 cells. Co-culture test was also performed in similar media.

The tumor cells (target (T)) was adjusted to 2×10⁵ cells/ml, and seeded on a 48-well treated culture plate at 500 μl/well (1×10⁵ cells/well). CAR-T (effector (E)) was diluted in media to adjust the E:T ratio to 4:1, 2:1, or 1:1.

For the E:T ratio of 4:1, CAR-T was adjusted to 8×10⁵ cells/nil and seeded on a 48-well treated culture plate at 500 μl/well (4×10⁵ cells/well) on which the tumor cells had been seeded.

For the E:T ratio of 2:1, also, CAR-T was adjusted to 4×10⁵ cells/ml and seeded on a 48-well treated culture plate at 500 μl/well (2×10⁵ cells/well) on which the tumor cells had been seeded.

For the E:T ratio of 1:1, CAR-T was adjusted to 2×10⁵ cells/ml and seeded on a 48-well treated culture plate at 500 μl/well (1×10⁵ cells/well) on which the tumor cells had been seeded.

Co-culture was performed for 4 days. CAR-T 006 and mock-T cells were subjected to co-culture in the same manner, and a control group consisting of the same number of tumor cells only to be cultured was prepared (CAR-T non-administered group). In addition, a group consisting of CAR-T cells only to be cultured for 4 days was prepared.

4 days after the initiation of co-culture, the cells were peeled from the wells using Accutase (Innovative Cell Technologies, Inc.) and the collected cells were centrifuged at 1,500×g for 5 minutes. To the centrifuged cells, 5 μl of APC Anti-Human CD3 antibody (Miltenyi Biotec) and 5 μl of PE anti-human Ganglioside GD2 antibody were added to prepare a suspension, and the suspension was subjected to antibody labeling reaction at 4° C. under shading conditions for 20 minutes. Thereafter, the cells were washed with 500 μl of D-PBS, and precipitated via centrifugation. After the supernatant was removed, the cells were re-suspended in 400 μl of D-PBS. The sample further supplemented with 50 μl of CountBright absolute counting beads (Invitrogen) was assayed using FACSCalibur (BD Biosciences), the obtained data were analyzed using FlowJo (BD Biosciences), and the number of GD2-positive tumor cells was calculated based on the number of counting beads.

When the cell population consisting of the CAR-T cells subjected to culture partially overlaps with the tumor cell population, in the flow cytometric analysis, the number of cells in the group consisting of the CAR-T cells was subtracted to correct the number of tumor cells.

In accordance with the formula below, the tumor cell proliferation rate (%) of CAR-T or mock-T cells was calculated to provide, an index of antitumor activity of CAR-T cells.

Tumor cell proliferation rate (%)=the number of tumor cells in the CAR-T administered group/the number of tumor cells in the CAR-T non-administered group×100

Lower tumor cell proliferation rate (%) indicates higher antitumor activity of CAR-T cells.

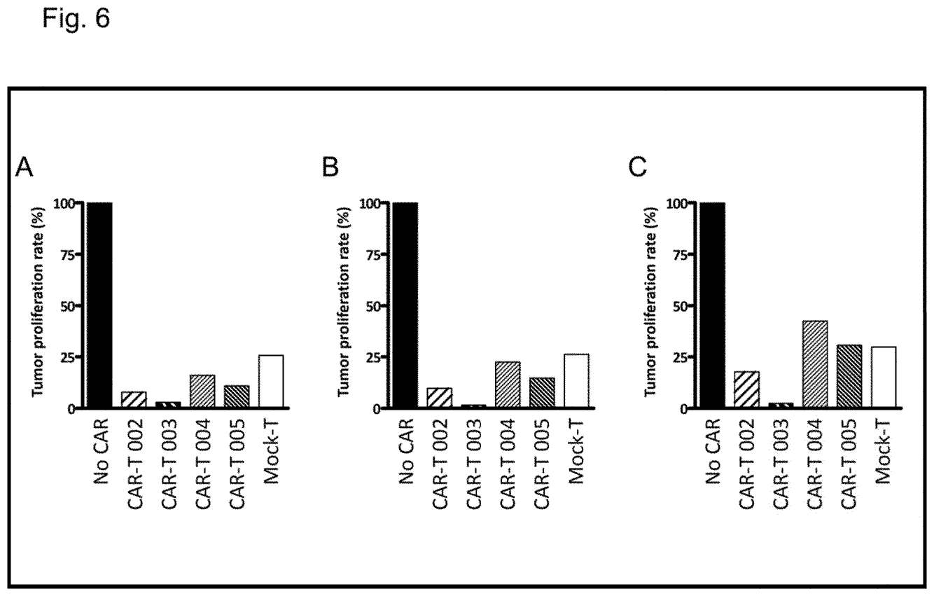

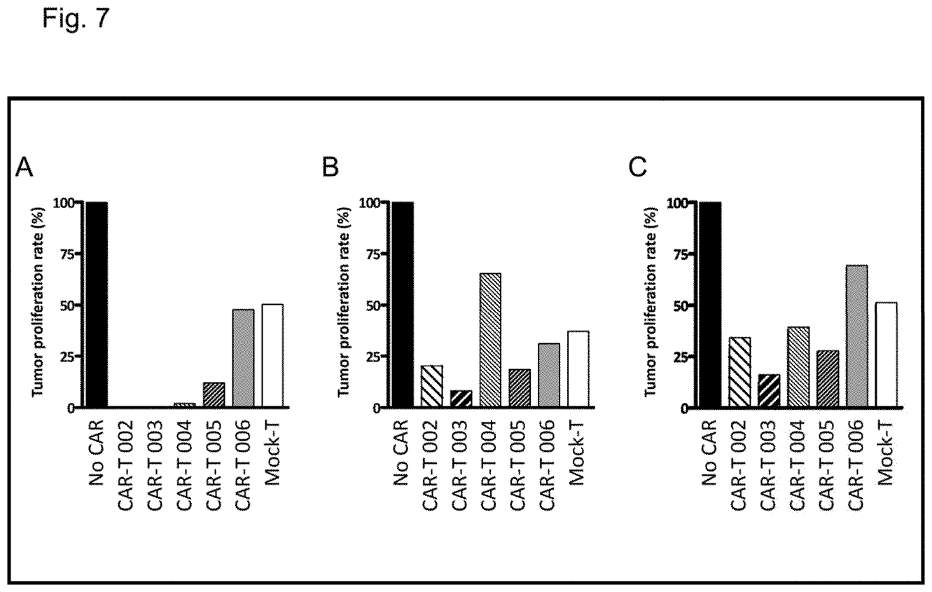

FIG. 6 shows antitumor activity of CAR-T 002 to CAR-T 005 or mock-T cells derived from Donor-1 against neuroblastoma cell line SH-SY5Y and FIG. 7 shows antitumor activity of CAR-T 002 to CAR-T 006 derived from Donor-2 against neuroblastoma cell line SH-SY5Y. Antitumor activity is shown in the charts in terms of the tumor cell proliferation rates (%) relative to the tumor cell proliferation rate exhibited by the CAR-T non-administered group (No CAR, 100%). As a result, the effects of CAR-T 002 to CAR-T 0005 to kill SH-SY5Y were confirmed, and the effects became lower in the order of CAR-T 003, CAR-T 002, CAR-T 005, and CAR-T 004.

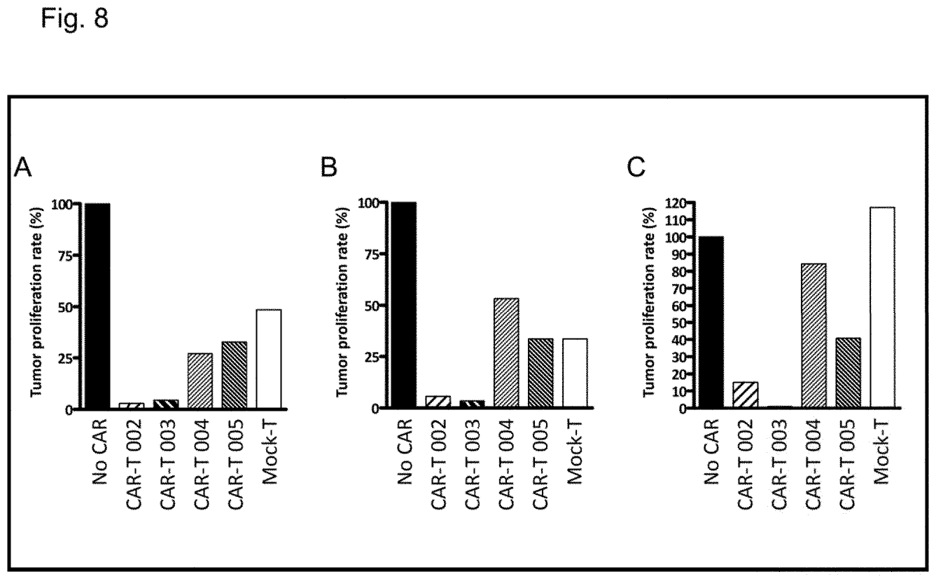

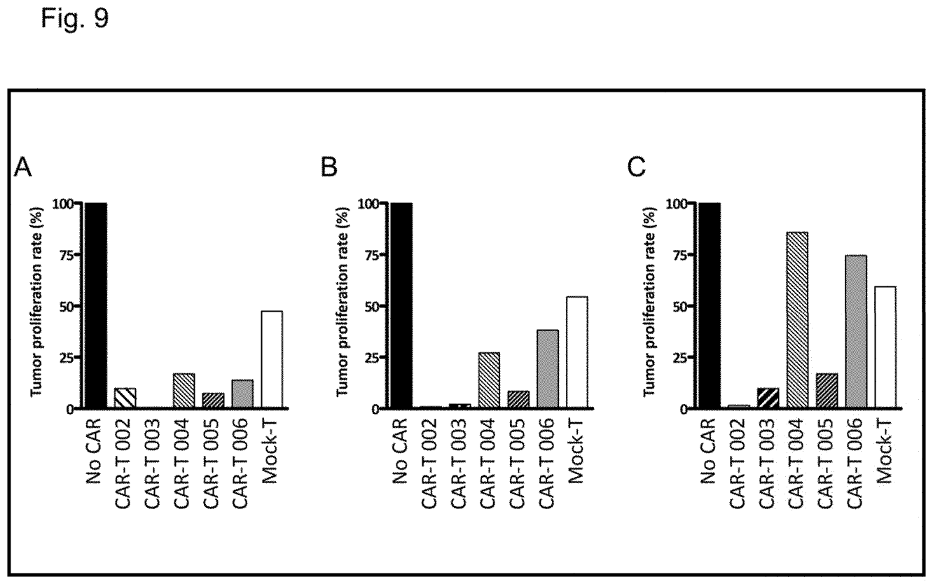

FIG. 8 shows antitumor activity of CAR-T 002 to CAR-T 005 or mock-T cells derived from Donor-1 against neuroblastoma cell line NB-1 and FIG. 9 shows antitumor activity of CAR-T 002 to CAR-T 006 or mock-T cells derived from Donor-2 against the neuroblastoma cell line NB-1. As a result, the effects of CAR-T 002 to CAR-T 005 to kill NB-1 were confirmed, the effects became lower in the order of CAR-T 003, CAR-T 002, CAR-T 005, and CAR-T 004, and the effects of CAR-T 002 and CAR-T 003 were higher than those of CAR-T 006 and mock-T cells.

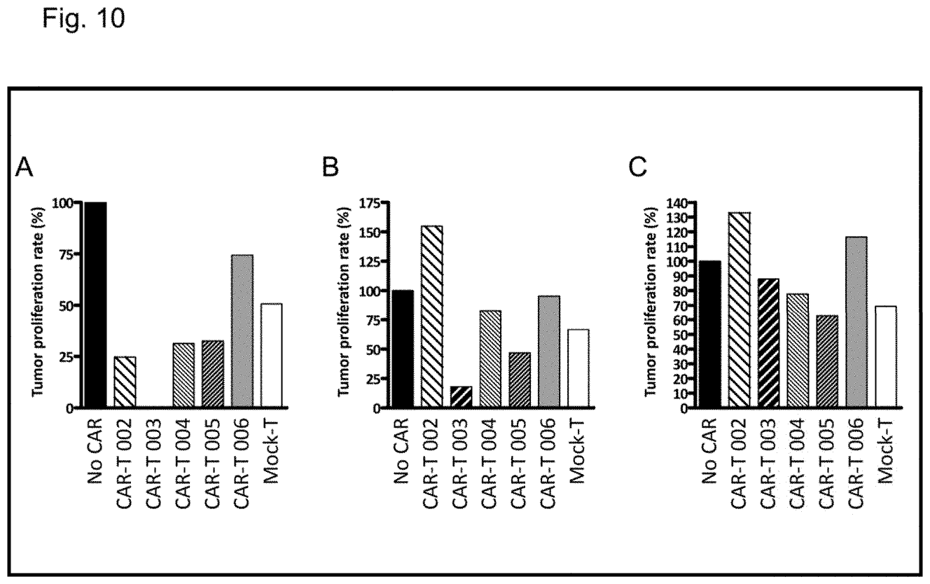

FIG. 10 shows antitumor activity of CAR-T 002 to CAR-T 006 or mock-T cells derived from Donor-2 against neuroblastoma cell line IMR-32. As a result, the effects of CAR-T 002 to CAR-T 005 to kill IMR-32 were confirmed. The effects of CAR-T 003 were particularly high.

In this example, antitumor activity of ligand-type CAR-T 002 and CAR-T 003 against neuroblastoma cells was found to be superior to that of scFv-type CAR-T 004 and CAR-T 005. As a result of comparison between CAR-T002 and CAR-T 003, antitumor activity of CAR-T 003 was considered to be slightly higher than that of CAR-T 002.

### Example 4

### Preparation of Plasmids for ALK (ALK 001), LTK (LTK 001), FAM150A (Ligand 001), FAM150B (Ligand 002 to Ligand 024) Mammalian Cell Expression Artificial Synthesis of ALK Gene

A sequence comprising: a 953- to 1009-bp sequence (SEQ ID NO: 17), which is a part of the translation region of ALK (NCBI Accession Number: NM_004304.4) encoding a region comprising a secretory signal: a 2894- to 4042-bp sequence (SEQ ID NO: 18), which is also a part of the translation region; a sequence encoding an octahistidine tag (SEQ ID NO: 19); and a sequence encoding a PA tag (SEQ ID NO: 20) ligated tandemly from the 5′ terminus was designed and codon-optimized for human. A DNA molecule comprising a Kozak sequence (SEQ ID NO: 21) at the 5′ terminus and 2 stop codons ligated to the 3′ terminus were then artificially synthesized (Kozak-ALK-His-PA) (synthesis was commissioned to Eurofins Genomics K.K., SEQ ID NO: 22).

### Artificial Synthesis of LTK Gene

A sequence comprising: a 179- to 1450-bp sequence (SEQ ID NO: 23), which is a part of the translation region of LTK (NCBI Accession Number: NM_002344.5); a sequence encoding an octahistidine tag (SEQ ID NO: 19): and a sequence encoding a PA tag (SEQ ID NO: 20) ligated tandemly from the 5′ terminus was designed and codon-optimized for human, and then a DNA molecule comprising a Kozak sequence (SEQ ID NO: 21) at the 5′ terminus and 2 stop codons ligated to the 3′ terminus was artificially synthesized (Kozak-LTK-His-PA) (synthesis was commissioned to Eurofins Genomics K.K., SEQ ID NO: 24).

### Artificial Synthesis of IgG1 Fe Gene

A sequence comprising: a sequence encoding an HRV3C protease cleavage sequence (SEQ ID NO: 25): a sequence encoding an Fc sequence (SEQ ID NO: 26) containing an IgG1-derived hinge region; a sequence encoding a G4S linker (SEQ ID NO: 27); a sequence encoding an octahistidine tag (SEQ ID NO: 19); and a sequence encoding a PA tag (SEQ ID NO: 20) ligated tandemly from the 5′ terminus was designed and codon-optimized for human, and then a DNA molecule comprising a Kozak sequence (SEQ ID NO: 21) at the 5′ terminus and 2 stop codons ligated to the 3′ terminus was artificially synthesized (Kozak-HRV3C-Fc-His-PA) (synthesis was commissioned to Eurofins Genomics K.K., SEQ ID NO: 28).

### Artificial Synthesis of FAM150A Gene

A sequence comprising: a sequence of a translation region (206 to 592 bp; SEQ ID NO: 1) of FAM150A (NCBI Accession Number: NM_207413.3): a sequence encoding an HRV3C protease cleavage sequence (SEQ ID NO: 25); and a sequence encoding an octahistidine tag (SEQ ID NO: 19) ligated tandemly from the 5′ terminus was designed and codon-optimized for human, and then a DNA molecule comprising a Kozak sequence (SEQ ID NO: 21) at the 5′ terminus and 2 stop codons ligated to the 3′ terminus was artificially synthesized (Kozak-FAM150A-HRV3C-His) (synthesis was commissioned to Eurofins Genomics K.K., SEQ ID NO: 29).

### Artificial Synthesis of FAM150B Gene

A sequence comprising: a sequence of a translation region (354 to 809 bp; SEQ ID NO: 3) of FAM150B (NCBI Accession Number: NM_001002919.2); a sequence encoding an HRV3C protease cleavage sequence (SEQ ID NO: 25): and a sequence encoding an octahistidine tag (SEQ ID NO: 19) ligated tandemly from the 5′ terminus was designed and codon-optimized for human, and then a DNA molecule comprising a Kozak sequence (SEQ ID NO: 21) at the 5′ terminus and 2 stop codons ligated to the 3′ terminus was artificially synthesized (Kozak-FAM150B-HRV3C-His) (synthesis was commissioned to Eurofins Genomics K.K., SEQ ID NO: 30).

### Preparation of ALK 001 and LTK 001 Plasmids

With the use of the artificial gene encoding ALK (Kozak-ALK-His-PA; SEQ ID NO: 22) as a template and primers represented by SEQ ID NO: 31 and SEQ ID NO: 32 (synthesis thereof was commissioned to Eurofins Genomics K.K.), a DNA sequence of an ALK-encoding region from the Kozak sequence was amplified by PCR (Kozak-ALK).

Further, with the use of the artificial gene encoding LTK (Kozak-LTK-His-PA; SEQ ID NO: 24) as a template and primers represented by SEQ ID NO: 33 and SEQ ID NO: 34 (synthesis thereof was commissioned to Eurofins Genomics K.K.), a DNA sequence of an LTK-encoding region from the Kozak sequence was amplified by PCR (Kozak-LTK).

In addition, with the use of the artificial gene encoding IgG1 Fc (Kozak-HRV3C-Fc-His-PA: SEQ ID NO: 28) as a template and primers represented by SEQ ID NO: 35 and SEQ ID NO: 36 (synthesis thereof was commissioned to Eurofins Genomics K.K.), a region from the sequence encoding the HRV3C protease cleavage sequence to the stop codon (HRV3C-Fc-His-PA) was amplified by PCR.

As a result of PCRs above, an overlap sequence to be ligated to pcDNA3.4 (Thermo Fisher Scientific) was added to the 5′ side of Kozak-ALK and Kozak-LTK, and an overlap sequence to be ligated to HRV3C-Fc-His-PA was added to the 3′ side thereof. Also, an overlap sequence to be ligated to pcDNA3.4 was added to the 3′ side of HRV3C-Fc-His-PA. PCR was carried out using KAPA HiFi HotStart ReadyMix (2X)(KAPA BIOSYSTEMS) with a cycle consisting of (i) 95° C. for 2 minutes, (ii 98°) C for 20 seconds, (iii) 65° C. for 15 seconds, and (iv) 72° C. for 30 seconds, and a cycle of steps (ii), (iii), and (iv) was repeated 25 times.

With the use of NEBuilder HiFi DNA Assembly Master Mix (New England Biolabs), amplified Kozak-ALK and HRV3C-Fc-His-PA and Kozak-LTK and HRV3C-Fc-His-PA were ligated to pcDNA3.4 (Thermo Fisher Scientific) cleaved with XbaI (New England Biolabs) and AgeI (New England Biolabs) in accordance with the instructions of the kit. E. coli DH5a (Toyobo Co. Ltd.) cells were transformed using the ligated cyclic plasmid and cultured on an LB agar medium containing 100 μg/ml carbenicillin at 37° C. overnight.

The appeared colonies were further cultured in an LB liquid medium containing 100 μg/ml carbenicillin at 37° C. overnight. Plasmids were purified from the cultured E. coli cells using Wizard Plus SV Minipreps DNA Purification System (Promega), nucleotide sequences were determined, and plasmids in which target nucleotide sequence insertion was observed were obtained as ALK 001 and LTK 001. Nucleotide sequence analysis was commissioned to Macrogen Japan.

The amino acid sequence of the ALK 001P protein comprising an ALK translation region encoded by ALK 001 and the DNA sequence encoding the same are represented by SEQ ID NO: 37 and SEQ ID NO: 38, respectively.

The amino acid sequence of the LTK 001P protein comprising an LTK translation region encoded by LTK 001 and the DNA sequence encoding the same are represented by SEQ ID NO: 39 and SEQ ID NO: 40, respectively.

### Preparation of Ligand 001 Plasmid

First-phase PCR was performed using the FAM150A artificial gene (Kozak-FAM 150A-HRV3C-His; SEQ ID NO: 29) as a template and primers represented by SEQ ID NO: 41 and SEQ ID NO: 42 (synthesis thereof was commissioned to Eurofins Genomics K.K.). Second-phase PCR was then performed using the first-phase PCR product as a template and primers represented by SEQ ID NO: 43 and SEQ ID NO: 42 (synthesis thereof was commissioned to Eurofins Genomics K.K.). As a result of the two-phase PCRs, a DNA molecule comprising an overlap sequence to be ligated to pM-secSUMOstar (LifeSensors) and a DNA sequence encoding an HRV3C protease cleavage sequence added to the 5′ side and a stop codon and an overlap sequence to be ligated to pM-secSUMOstar (LifeSensors) added to the 3′ side of the sequence encoding a region of 50 to 129 residues of FAM150A were amplified.

PCR was carried out using KAPA HiFi HotStart ReadyMix (2X) (KAPA BIOSYSTEMS) with a cycle consisting of (i) 95° C. for 2 minutes, (ii) 98° C. for 20 seconds, (iii) 65° C. for 15 seconds, and (iv) 72° C. for 15 seconds. A cycle of steps (ii), (iii), and (iv) was repeated 25 times in the first-phase PCR and 13 times in the second-phase PCR. With the use of NEBuilder HiFi DNA Assembly Master Mix (New England Biolabs), the second-phase PCR product was ligated to pM-secSUMOstar (LifeSensors) cleaved with BsmBI (New England Biolabs) in accordance with the instructions of the kit. Thus, a DNA sequence (DNA sequence: SEQ ID NO: 44; amino acid sequence: SEQ ID NO: 45) encoding a fusion protein comprising an Ig κ light chain secretory signal, a hexahistidine tag, and SUMOstar ligated tandemly to the 5′ side of the inserted DNA sequence was cloned in-frame. E. coli DH5α (Toyobo Co. Ltd.) cells were transformed using the ligated cyclic plasmid and cultured on an LB agar medium containing 100 μg/ml carbenicillin at 37° C. overnight.

The appeared colonies were further cultured in an LB liquid medium containing 100 μg/ml carbenicillin at 37° C. overnight. Plasmids were purified from the cultured E. coli cells using Wizard Plus SV Minipreps DNA Purification System (Promega), nucleotide sequences were determined, and a plasmid in which target nucleotide sequence insertion had been observed was obtained as Ligand 001. Nucleotide sequence analysis was commissioned to Macrogen Japan.

The amino acid sequence of the Ligand 001P protein comprising a region of 50 to 129 residues of FAM150A encoded by Ligand 001 and the DNA sequence encoding the same are represented by SEQ ID NO: 46 and SEQ ID NO: 47, respectively.

### Preparation of Ligand 002 Plasmid

First-phase PCR was performed using the FAM150B artificial gene (Kozak-FAM150B-HRV3C-His; SEQ ID NO: 30) as a template and primers represented by SEQ ID NO: 48 and SEQ ID NO: 49 (synthesis thereof was commissioned to Eurofins Genomics K.K.). Second-phase PCR was performed using the first-phase PCR product as a template and primers represented by SEQ ID NO: 43 and SEQ ID NO: 49 (synthesis thereof was commissioned to Eurofins Genomics K.K.). Ligand 002 was prepared in the same manner as with the case of Ligand 001.

The amino acid sequence of the Ligand 002P protein comprising a region of 71 to 152 residues of FAM150B encoded by Ligand 002 and the DNA sequence encoding the same are represented by SEQ ID NO: 50 and SEQ ID NO: 51, respectively.

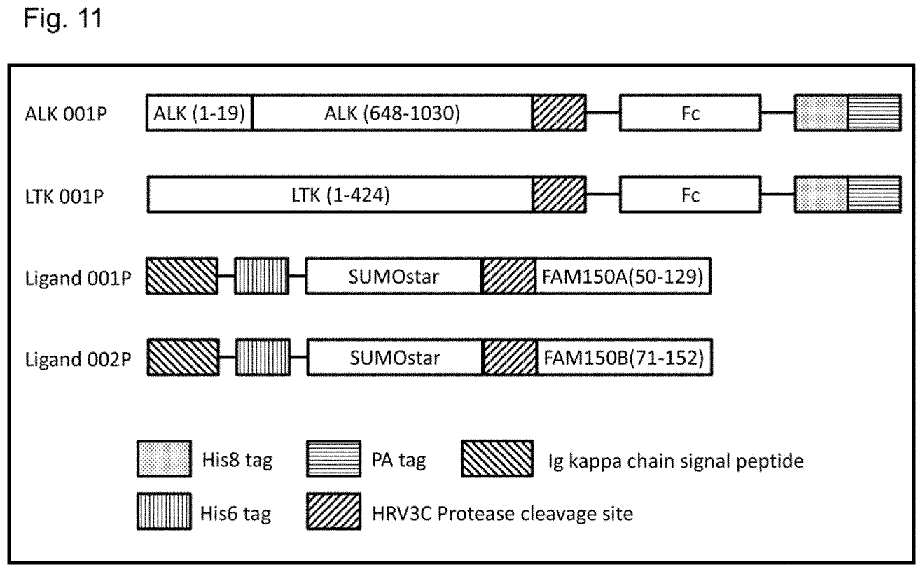

Constitutions of the proteins prepared in this example (ALK 001P, LTK 001P, Ligand 001P, and Ligand 002P) are schematically shown below and in FIG. 11. Numerical values in parentheses indicate amino acid positions.

### ALK 001P:

ALK (1-19)-ALK (648-1030)-HRV3C protease cleavage sequence-Fc sequence containing hinge region-G4S linker-octahistidine tag-PA tag

### LTK 001P:

LTK (1-424)-HRV3C protease cleavage sequence-Fc sequence containing hinge region-G4S linker-octahistidine tag-PA tag

### Ligand 001P:

Ig κ light chain secretory signal-hexahistidine tag-SUMOstar-HRV3C protease cleavage sequence-FAM150A (50-129)

### Ligand 002P:

Ig κ light chain secretory signal-hexahistidine tag-SUMOstar-HRV3C protease cleavage sequence-FAM150B (71-152)

### Example 5

### Preparation of ALK 001P, LTK 001P, Ligand 001P, and Ligand 002P

### 5-1. Preparation of ALK 001P

ALK 001P expression

ALK 001P was expressed using the plasmid ALK 001 prepared in Example 4 and Expi293 Expression System (Thermo Fisher Scientific, mammalian cell expression system).

Expi293 cells cultured in Expi293 Expression Medium were diluted to 2.9×10⁶ cells/ml with Expression Medium, the resulting culture solution (2.55 ml) was prepared on a 6-well plate, and 3 μg of ALK 001 was transfected into the Expi293 cells in accordance with the instructions. Culture was carried out in the presence of 8% CO₂ at 125 rpm and 37° C. 23 hours after transfection, Enhancers 1 and 2 included in the kit were added in amounts of 15 μl and 150 μl, respectively, and culture was continued. The culture supernatant was collected via centrifugation 4 days after transfection. The collected supernatant was cryopreserved at −30° C. to −80° C. before purification.

### ALK 001P Purification

After the culture supernatant was thawed, the buffer was exchanged with 50 mM Tris-HCl (pH 8.0) and 500 mM NaCl using PD-10 (GE Healthcare Japan). The resultant was mixed with 0.5 ml of Ni Sepharose 6 Fast Flow (GE Healthcare Japan) in a chromatography column, the mixture was stirred at 4° C. for 2.5 hours, and ALK 001P was allowed to adsorb to resin. After the resin was washed with 50 mM Tris-HCl (pH 8.0), 500 mM NaCl, and 20 mM imidazole, ALK 001P was eluted with the aid of 50 mM Tris-HCl(pH 8.0), 500 mM NaCl, and 500 mM imidazole.

The resulting eluate was subjected to separation using Superdex 200 increase 10/300 GL (GE Healthcare Japan) connected to AKTA explorer (GE Healthcare Japan) at a flow rate of 0.8 ml/min, and the main peak fraction was collected and used as a sample for SPR-based interaction analysis.

### 5-2. Preparation of LTK 001P

LTK 001P expression

LTK 001P was expressed using the plasmid LTK 001 prepared in Example 4 and Expi293 Expression System (Thermo Fisher Scientific).

Expi293 cells cultured in Expi293 Expression Medium were diluted to 2.9×10⁶ cells/ml with Expression Medium, the resulting culture solution (25.5 ml) was prepared in a 125-ml flask, and 30 μg of LTK 001 was transfected into the Expi293 cells in accordance with the instructions. 21 hours after transfection, Enhancers 1 and 2 included in the kit were added in amounts of 0.15 ml and 1.5 ml, respectively, and culture was continued. Culture was carried out in the presence of 8% CO₂ at 125 rpm and 37° C. The culture supernatant was collected via centrifugation 8 days after transfection. The collected supernatant was cryopreserved at −30° C. to −80° C. before purification.

LTK 001P purification

After the culture supernatant was thawed, the supernatant was diluted to 2-fold with 50 mM Tris-HCl(pH 8.0) and 500 mM NaCl, the resultant was mixed with 3 ml of Ni Sepharose 6 Fast Flow (GE Healthcare Japan), the mixture was stirred at 4° C. for 1.5 hours, and LTK 001P was allowed to adsorb to resin. Thereafter, the suspension was transferred to an empty chromatography column, the resin was washed with 50 mM Tris-HCl (pH 8.0), 500 mM NaCl, and 20 mM imidazole, and LTK 001P was eluted with the aid of 50 mM Tris-HCl (pH 8.0), 500 mM NaCl, and 500 mM imidazole.

The resulting eluate was subjected to separation using Superdex 200 increase 10/300 GL (GE Healthcare Japan) connected to AKTA explorer (GE Healthcare Japan) at a flow rate of 0.8 mil/min, and the main peak fraction was collected and used as a sample for SPR-based interaction analysis.

### 5-3. Preparation of Ligand 001P and Ligand 002P

### Ligand 001P and Ligand 002P Expression

Ligand 001P and Ligand 002P were expressed using Ligand 001 and Ligand 002 prepared in Example 4 and the Expi293 Expression System (Thermo Fisher Scientific).

Expi293 cells cultured in Expi293 Expression Medium were diluted to 2.9×10⁶ cells/ml with Expression Medium, the resulting culture solution (25.5 ml) was prepared in a 125-ml flask, and 30 μg of Ligand 001 or Ligand 002 were transfected into the Expi293 cells in accordance with the instructions. 19 hours after transfection, Enhancers 1 and 2 included in the kit were added in amounts of 0.15 ml and 1.5 ml, respectively, and culture was continued. Culture was carried out under 8% CO₂ at 125 rpm and at 37° C. The culture supernatant was collected via centrifugation 4 days after transfection.

Ligand 001P and Ligand 002P purification

The culture supernatant was diluted to 2-fold with 50 mM Tris-HCl (pH 8.0) and 500 mM NaCl, the resultant was mixed with 1 ml of cOmplete™ His-Tag Purification Resin(Roche Diagnostics), the mixture was stirred at 4° C. for 1.5 hours, and Ligand 001P or Ligand 002P was allowed to adsorb to resin. Thereafter, the suspension was transferred to an empty chromatography column, the resin was washed with 50 mM Tris-HCl (pH 8.0), 500 mM NaCl, and 20 mM imidazole, and Ligand 001P or Ligand 002P was eluted with the aid of 50 mM Tris-HCl (pH 8.0), 500 mM NaCl, and 500 mM imidazole.

The resulting eluate was subjected to separation using Superdex 75 10/300 GL (GE Healthcare Japan) connected to AKTA explorer (GE Healthcare Japan) at a flow rate of 0.7 ml/min, and the main peak fraction was collected and used as a sample for SPR-based interaction analysis.

### Example 6

### Verification of Binding of ALK 001P and LTK 001P to Ligand 001P and Ligand

### 002P Via Surface Plasmon Resonance Analysis

Whether or not ALK 001P and LTK 001P obtained in Example 5 had bound to Ligand 001P and Ligand 002P was examined using Biacore 8K instrument (GE Healthcare Japan) by the surface plasmon resonance (SPR) technique.

### Immobilization of ALK 001P and LTK 001P on Sensor Chip

ALK 001P contained at 4.8 μg/ml (calculated based on the molecular weight without sugar chain) and LTK 001P contained at 10 μg/ml (calculated based on the molecular weight without sugar chain) in HBS-P+(10 mM HEPES (pH 7.4), 150 mM NaCl, 0.05% (v/v) Surfactant P20) were introduced at a flow rate of 10 Id/min for 90 seconds and immobilized on the surface coated with Protein A of Series S Sensor Chip Protein A (GE Healthcare Japan). The immobilized amount of ALK001P was 830 to 990 RU, and the immobilized amount of LTK 001P was 670 to 790 RU.

### Acquisition of Bond Dissociation Curve

Six concentrations of Ligand 001P at 2-fold dilution with HBS-P+ from 19.8 nM and six concentrations thereof at 2-fold dilution with HBS-P+ from 297 nM were prepared separately. Six concentrations of Ligand 002P at 2-fold dilution with HBS-P+ from 19.9 nM was also prepared.

Ligand 001P and Ligand 002P at various concentrations and HBS-P+(concentration 0) were introduced on the surface of the immobilized ALK 001P or LTK 001P and on the untreated surface coated with Protein A (baseline) at a flow rate of 30 μl/min for 120 seconds, in the combinations as shown in Table 2, and the binding curves were recorded. Subsequently, HBS-P+ was introduced on the surface of the immobilized ALK 001P or LTK 001P and on the untreated surface coated with Protein A (baseline) at a flow rate of 30 μl/min for 600 seconds, and the dissociation curves were recorded.

ALK 001P and LTK 001P were dissociated (i.e., regenerated) from Protein A coating the sensor chip surface (i.e., regeneration) by injecting 10 mM Glycine-HCl (pH 1.6) at a flow rate of 30 μl/min for 30 seconds. The bond dissociation curve modified by subtracting the baseline value was prepared using Biacore8K Evaluation Software (GE Healthcare Japan).

TABLE 2

| | ALK 001P | LTK 001P |
|---|---|---|
| Ligand 001P | 6 concentration levels | 6 concentration levels |
| | from 297 nM | from 19.8 nM |
| Ligand 002P | 6 concentration levels | 6 concentration levels |
| | from 19.9 nM | from 19.9 nM |

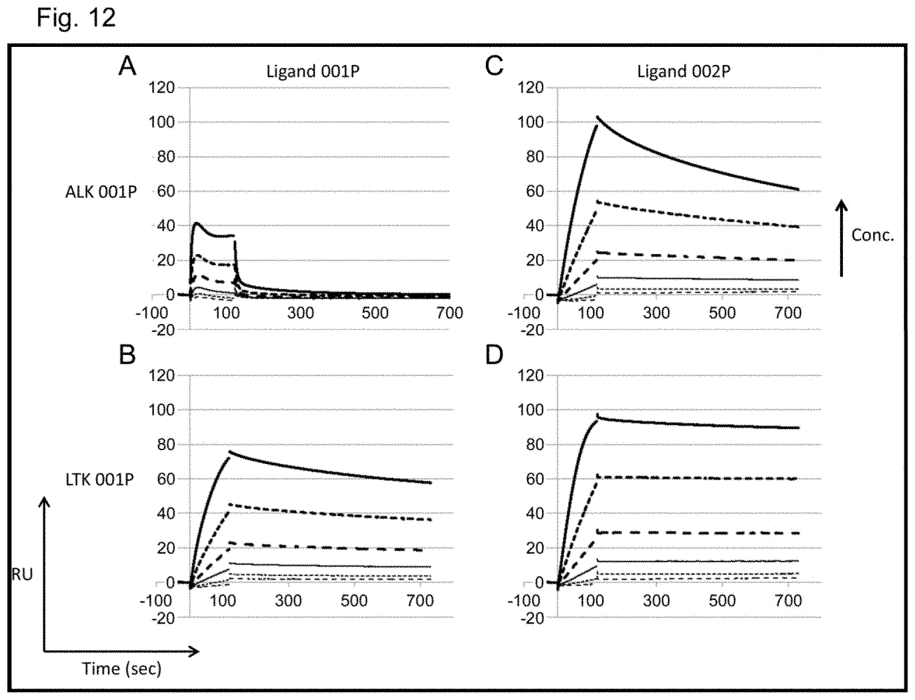

FIG. 12 shows the results of verification of binding of ALK 001P and LTK 001P to Ligand 001P and Ligand 002P via surface plasmon resonance analysis. Ligand 001P shows a higher binding ability to LTK 001P than to ALK 001P. Ligand 002P is slowly detached from ALK 001P and LTK 001P, indicating a high binding ability thereto.

### Example 7

### Preparation of FAM150A Truncated CAR-Expressing Plasmid (CAR 007: FAM150ATr-28z) and FAM150B Truncated CAR-Expressing Plasmid (CAR 008; FAM150BTr-28z)

With the use of CAR 002 or CAR 003 prepared in Example 1 as a template, FAM150A truncated (CAR 007) and FAM150B truncated (CAR 008) CAR-expressing plasmids were prepared via inverse-PCR.

As PCR primers used for inverse-PCR, primers represented by SEQ ID NO: 52 and SEQ ID NO: 53 (for CAR 002 linearization) and primers represented by SEQ ID NO: 54 and SEQ ID NO: 55 (for CAR 003 linearization) were designed and synthesized (synthesis was commissioned to Eurofins Genomics K.K.).

CAR 002 or CAR 003 was adjusted to 50 ng/μl and used as a template, and the concentration of each PCR primer was adjusted to 0.2 μM in the reaction solution. Inverse-PCR was performed using KOD-Plus-Mutagenesis Kit (Toyobo Co. Ltd.) with the reaction composition designated in the instructions of the kit and with a cycle consisting of (i) 94° C. for 2 minutes, (ii) 98° C. for 10 seconds, and (iii) 68° C. for 7 minutes, and a cycle of steps (ii) and (iii) was repeated 10 times.

After the PCR reaction, an aliquot of the sample was separated via 1% agarose gel electrophoresis, amplification of a linear plasmid of the size of interest was confirmed, the remaining sample after PCR was treated with DpnI in accordance with the instructions of the kit, and methylated template plasmid CAR 002 or CAR 003 was cleaved and removed. Thereafter, the linear plasmid was phosphorylated with T4 Polynucleotide Kinase included in the kit and self-ligated to form a cyclic plasmid. Thereafter, E. coli DH5a.(Toyobo Co. Ltd.) cells were transformed using the cyclic plasmid and then cultured on an LB agar medium containing 100 μg/ml carbenicillin for about 16 hours.

The appeared colonies were further cultured in an LB liquid medium containing 100 μg/ml carbenicillin for about 16 hours. Plasmids were purified from the cultured E. coli cells using QIAprep Spin Miniprep Kit (Qiagen), nucleotide sequences were determined, and plasmids in which target nucleotide sequence deletion was observed were obtained as a FAM150A truncated CAR-expressing plasmid (CAR 007; FAM150ATr-28z) and a FAM150B truncated CAR-expressing plasmid (CAR 008; FAM150BTr-28z).

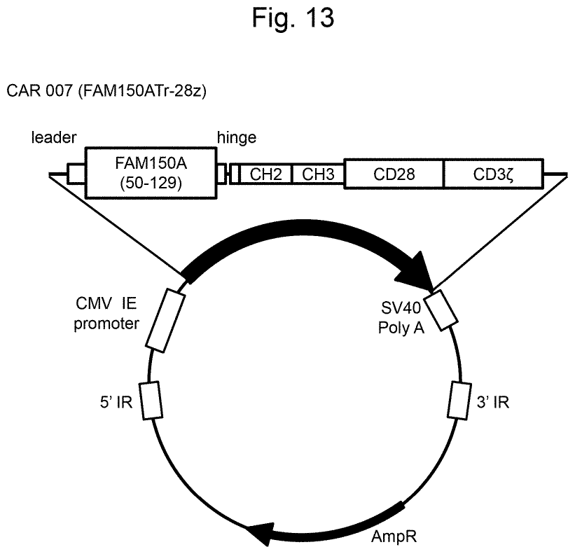

FIG. 13 shows a vector map of CAR 007 (FAM150ATr-28z) and the DNA sequence of a translation region of CAR 007 is represented by SEQ ID NO: 56.

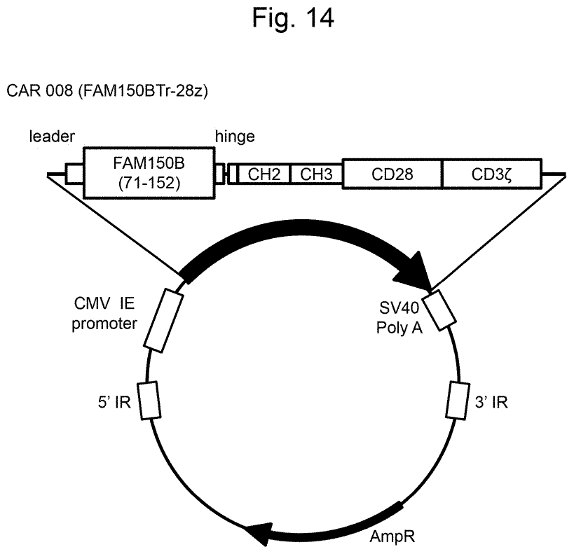

FIG. 14 shows a vector map of CAR 008 (FAM150BTr-28z) and the DNA sequence of a translation region of CAR 008 is represented by SEQ ID NO: 57.

### Example 8

### Antitumor Activity of FAM150B Truncated CD28-Type CAR-T

In accordance with the method of CAR-T cell culture and proliferation in Example 2, CAR-T cells were prepared by introducing genes into PBMCs derived from 2 healthy adult donors using the CAR-expressing plasmids CAR 003, CAR 008, or CAR 006.

CAR-T cells obtained by electrical introduction into PBMCs using CAR 003, CAR 008, or CAR 006 and T cell culture and proliferation are described herein as follows.
CAR-T 003: FAM150B CD28-type CAR-T (FAM150B-28z CAR-T)
CAR-T 006: CD19 scFv CD28-type CAR-T(CD19-28z CAR-T)
CAR-T 008: FAM150B truncated CD28-type CAR-T (FAM150BTr-28z CAR-T)

Table 3 shows CAR expression rates in the CAR-T cells obtained by introduction of CAR-expressing plasmids into PBMCs derived from 2 healthy adult donors and culture.

The amino acid sequence of CAR 003 and the nucleotide sequence encoding the same are represented by SEQ ID NO: 14 and SEQ ID NO: 10, respectively.

The amino acid sequence of CAR 008 and the nucleotide sequence encoding the same are represented by SEQ ID NO: 58 and SEQ ID NO: 57, respectively.

TABLE 3

| Donor | Plasmid | CAR-T | Expression rate |
|---|---|---|---|
| Donor-1 | CAR 003 | CAR-T 003 | 23.6 |
| | CAR 008 | CAR-T 008 | 39.0 |
| | CAR 006 | CAR-T 006 | 48.9 |
| Donor-2 | CAR 003 | CAR-T 003 | 20.8 |
| | CAR 008 | CAR-T 008 | 27.8 |
| | CAR 006 | CAR-T 006 | 27.5 |

### Measurement of Antitumor Activity of CAR-T

In order to measure antitumor activity of CAR-T 003, CAR-T 008, and CAR-T 006 obtained above, co-culture with solid tumor cells was conducted. In this example, SH-SY5Y and IMR-32 were used as target tumor cells, and activity evaluation was performed in accordance with the method of Example 3. In this example, in addition, the number of tumor cells measured using a flow cytometer when co-culture was initiated was designated to be 1, and the relative ratio of the number of GD2-positive tumor cells when co-culture was terminated is plotted to prepare the tumor cell proliferation curves.

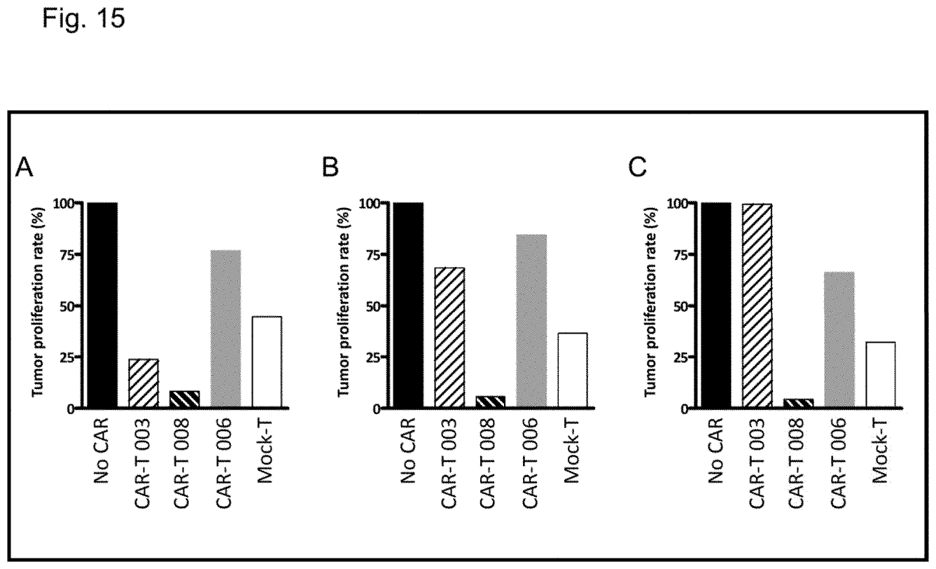

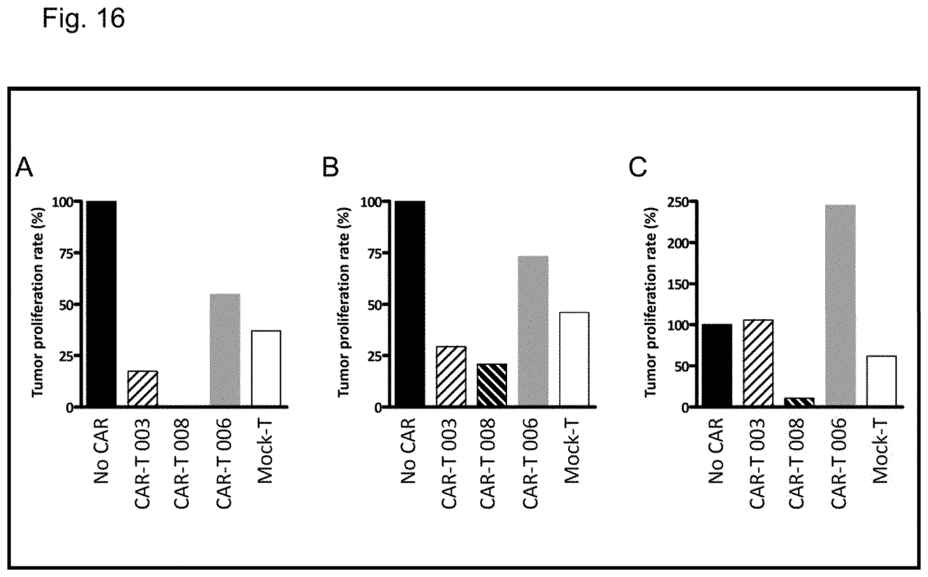

FIG. 15 and FIG. 16 each show antitumor activity of CAR-T 003. CAR-T 008, and CAR-T 006 or mock-T cells derived from Donor-1 against SH-SY5Y or IMR-32. As a result, the effects of CAR-T 003 and CAR-T 008 to kill SH-SY5Y and IMR-32 were verified. In particular, the effects of CAR-T 008 were higher than those of CAR-T 003 and also high at E:T of 1:1.

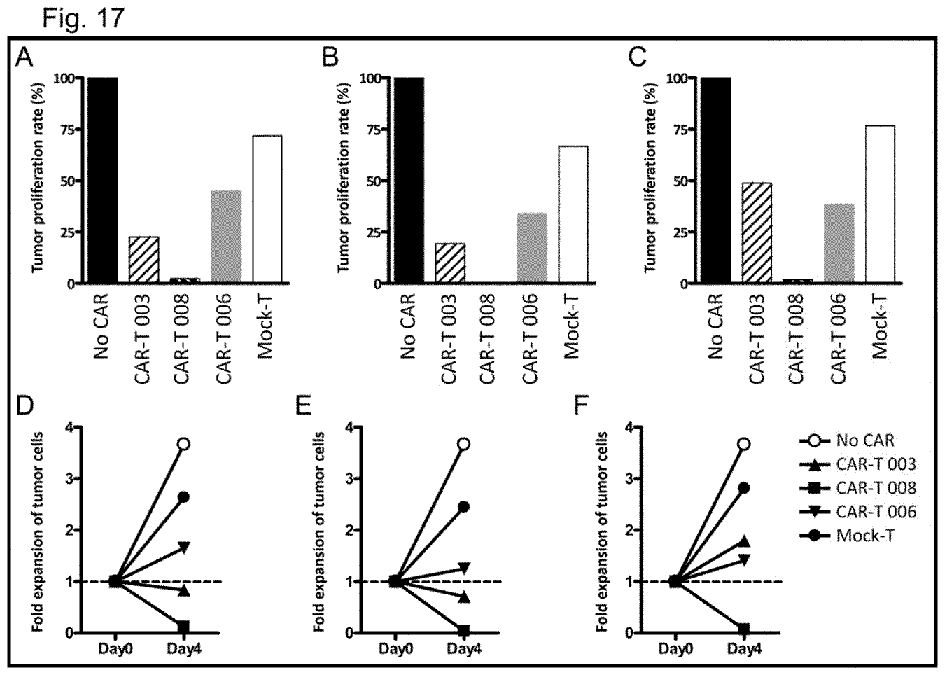

FIG. 17 shows antitumor activity of CAR-T 003. CAR-T 008, and CAR-T 006 or mock-T cells derived from Donor-2 against SH-SY5Y and the tumor cell proliferation curves. As a result, the effects of CAR-T 003 and CAR-T 008 to kill SH-SY5Y were verified. In particular, the effects of CAR-T 008 were higher than those of CAR-T 003 and also high at E:T of 1:1.

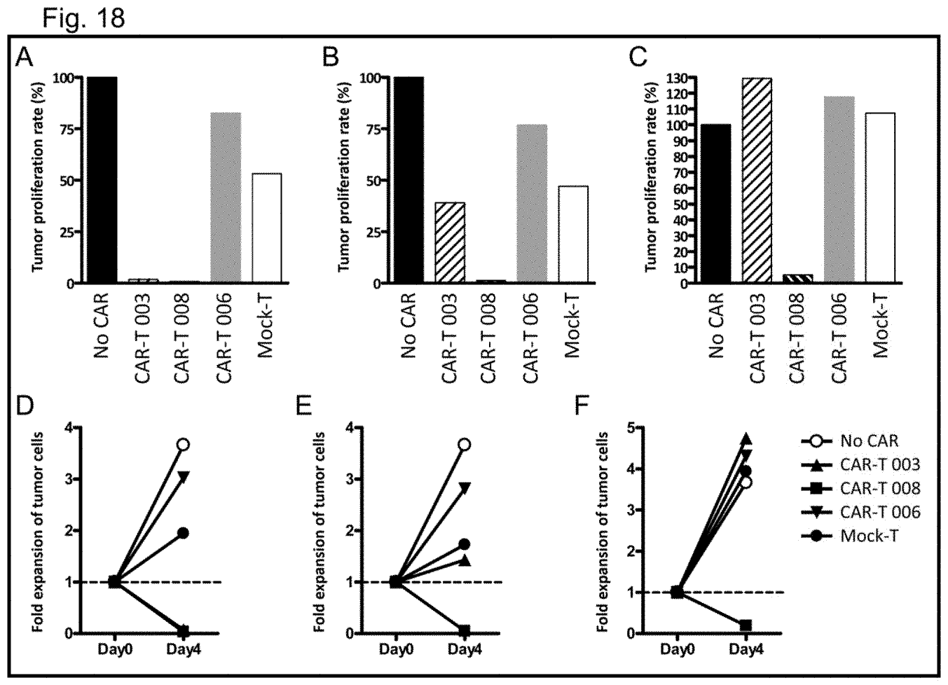

FIG. 18 shows antitumor activity of CAR-T 003, CAR-T 008, and CAR-T 006 or mock-T cells derived from Donor-2 against IMR-32 and the tumor cell proliferation curves. As a result, the effects of CAR-T 003 and CAR-T 008 to kill IMR-32 were verified. In particular, the effects of CAR-T 008 were higher than those of CAR-T 003 and also high at E:T of 1:1.

This example indicates that antitumor activity of FAM150B truncated CAR-T (CAR-T 008) is higher than that of FAM150B full-length CAR-T (CAR-T 003).

### Example 9

### Preparation of Ligand 003 Plasmid

In order to increase the expression level, a DNA sequence encoding Ligand 002P was introduced into pcDNA3.4.

With the use of Ligand 002 as a template and primers represented by SEQ ID NO: 59 and SEQ ID NO: 60 (synthesis thereof was commissioned to Eurofins Genomics K.K.), a region from the sequence encoding the Ig κ light chain secretory signal sequence to a sequence encoding a region of 71 to 152 residues of FAM150B (Ig kappa-SUMOstar-FAM150B (71-152)) was amplified by PCR. As a result of PCR, a DNA sequence comprising an overlap sequence to be ligated to pcDNA3.4 (Thermo Fisher Scientific) added to the 5′ side and a stop codon and an overlap sequence to be ligated to pcDNA3.4 (Thermo Fisher Scientific) added to the 3′ side of Ig kappa-SUMOstar-FAM150B (71-152) was amplified.

PCR was carried out using KAPA HiFi HotStart ReadyMix (2X) (KAPA BIOSYSTEMS) with a cycle consisting of (i) 95° C. for 2 minutes, (ii) 98° C. for 20 seconds, (iii) 65° C. for 15 seconds, and (iv) 72° C. for 15 seconds, and a cycle of steps (ii), (iii), and (iv) was repeated 25 times. With the use of NEBuilder HiFi DNA Assembly Master Mix (New England Biolabs), the amplified sequence was ligated to pcDNA3.4 (Thermo Fisher Scientific) cleaved with XbaI (New England Biolabs) and AgeI (New England Biolabs) in accordance with the instructions of the kit. E. coli DH5a (Toyobo Co. Ltd.) cells were transformed using the ligated cyclic plasmid and cultured on an LB agar medium containing 100 μg/ml carbenicillin at 37° C. overnight.

The appeared colonies were further cultured in a MMI liquid medium (20 mM Tris-HCl (pH 7.2), 1.25% (w/v) tryptone, 2.5% (w/v) yeast extract, 0.85% (w/v) NaCl, 0.4% (v/v) glycerol) containing 100 μg/ml carbenicillin at 37° C. for about 9.5 hours. Plasmids were purified from the cultured E. coli cells using Wizard Plus SV Minipreps DNA Purification System (Promega), nucleotide sequences were determined, and a plasmid in which target nucleotide sequence insertion was observed was obtained as Ligand 003. Nucleotide sequence analysis was commissioned to Macrogen Japan.

The amino acid sequence of the Ligand 003P protein containing 71 to 152 residues of FAM150B encoded by Ligand 003 and the DNA sequence encoding the same are the same as those of Ligand 002P (amino acid sequence: SEQ ID NO: 50, DNA sequence: SEQ ID NO: 51).

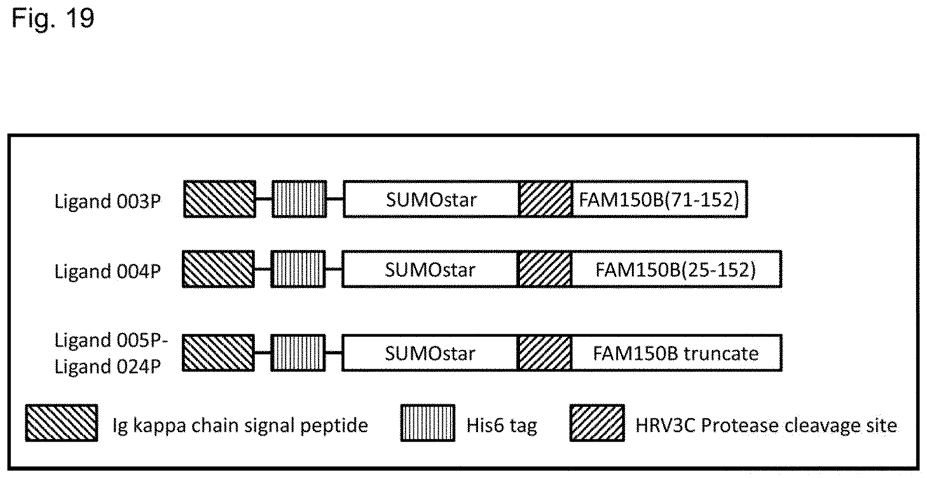

The constitution of Ligand 003P is schematically shown below and in FIG. 19. Numerical values in parentheses indicate amino acid positions.

### Ligand 003P:

Ig Kappa light chain secretory signal-hexahistidine tag-SUMOstar-HRV3C protease cleavage sequence-FAM150B (71-152)

### Example 10

### Preparation of Expression Plasmids for Various FAM150B Truncated Forms

### Preparation of Ligand 004 Plasmid

In order to prepare expression plasmids for various FAM150B truncated forms, Ligand 004 to be used as a template was prepared. First-phase PCR was carried out using the FAM150B artificial gene obtained in Example 4 (Kozak-FAM150B-HRV3C-His: SEQ ID NO: 30) as a template and primers represented by SEQ ID NO: 61 and SEQ ID NO: 49 (synthesis thereof was commissioned to Eurofins Genomics K.K.). Second-phase PCR was carried out using the first-phase PCR product as a template and primers represented by SEQ ID NO: 43 and SEQ ID NO: 49 (synthesis thereof was commissioned to Eurofins Genomics K.K.). Thereafter, in the same manner as in the case of Ligand 001, Ligand 004 was prepared.

The amino acid sequence of the Ligand 004P protein containing 25 to 152 residues of FAM150B encoded by Ligand 004 and the DNA sequence encoding the same are represented by SEQ ID NO: 62 and SEQ ID NO: 63, respectively.

### Preparation of Ligand 005 to Ligand 022 Plasmids

First-phase PCR was carried out using the combination of the template with the primers shown in Table 4 below (synthesis thereof was commissioned to Eurofins Genomics K.K.) to amplify DNA sequences encoding various FAM150B truncated forms. As a result of PCR, DNA sequences comprising an overlap sequence to be ligated to Ig kappa-SUMOstar-HRV3C added to the 5′ side and a stop codon and an overlap sequence to be ligated to pcDNA3.4 (Thermo Fisher Scientific) added to the 3′ side of DNA sequences encoding various FAM150B truncated forms were amplified.

At the same time, first-phase PCR was carried out using Ligand 004 as a template and primers represented by SEQ ID NO: 59 and SEQ ID NO: 84 (synthesis thereof was commissioned to Eurofins Genomics K.K.) to amplify a region from the DNA sequence encoding the Ig kappa light chain secretory signal sequence to the DNA sequence encoding the HRV3C protease cleavage sequence (Ig kappa-SUMOstar-HRV3C) by PCR. As a result of PCR, a DNA sequence comprising an overlap sequence to be ligated to pcDNA3.4 (Thermo Fisher Scientific) and a Kozak sequence added to the 5′ side was amplified.

Subsequently, the first-phase PCR products comprising DNA sequences encoding various FAM150B truncated forms were mixed with the first-phase PCR product comprising Ig kappa-SUMOstar-HRV3C. Second-phase PCR was then carried out using the primers represented by SEQ ID NO: 59 and SEQ ID NO: 60 (synthesis thereof was commissioned to Eurofins Genomics K.K.) to amplify a DNA sequence comprising both the PCR products ligated to each other.

PCR was carried out using KAPA HiFi HotStart ReadyMix(2X) (KAPA BIOSYSTEMS) with a cycle consisting of (i 95°) C for 2 minutes. (ii) 98° C. for 20 seconds,(iii) 65° C. for 15 seconds, and (iv) 72° C. for 15 seconds. In the first-phase PCR, a cycle of steps (ii), (iii), and (iv) was repeated 25 times, and in the second-phase PCR it was repeated 13 times. However, the first-phase PCR for amplifying a DNA fragment encoding a FAM150B truncated form when preparing Ligand 015 and Ligand 016 was carried out using KAPA HiFi HotStart ReadyMix(2X) (KAPA BIOSYSTEMS) with a cycle consisting of (i) 95° C. for 2 minutes, (ii) 98° C. for 20 seconds, (iii) 70.6° C. for 15 seconds, and (iv) 72° C. for 15 seconds, and a cycle of steps (ii), (iii), and (iv) was repeated 25 times.

With the use of NEBuilder HiFi DNA Assembly Master Mix (New England Biolabs), the second-phase PCR product was ligated to pcDNA3.4 (Thermo Fisher Scientific) cleaved with XbaI (New England Biolabs) and AgeI (New England Biolabs) in accordance with the instructions of the kit. E. coli DH5a (Toyobo Co. Ltd.) cells were transformed using the ligated cyclic plasmid and cultured on an LB agar medium containing 100 μg/ml carbenicillin at 37° C. overnight.

The appeared colonies were further cultured in an LB liquid medium containing 100 μg/ml carbenicillin at 37° C. overnight. Plasmids were purified from the cultured E. coli cells using FastGene Plasmid Mini Kit (Nippon Genetics Co., Ltd.), nucleotide sequences were determined, and plasmids in which target nucleotide sequence insertion was observed were obtained as Ligand 005 to Ligand 022. Nucleotide sequence analysis was commissioned to Macrogen Japan.

### Preparation of Ligand 023 and Ligand 024 Plasmids

A DNA sequence encoding a FAM150B truncated form was amplified via PCR using the template in combination with the primers shown in Table 4 below (synthesis thereof was commissioned to Eurofins Genomics K.K.). As a result of PCR, a DNA sequence, comprising an overlap sequence to be ligated to pcDNA3.4 (Thermo Fisher Scientific) and a Kozak sequence added to the 5′ side and a stop codon and an overlap sequence to be ligated to pcDNA3.4 (Thermo Fisher Scientific) added to the 3′ side of the DNA sequence encoding the FAM150B truncated form, was amplified.

PCR was carried out using KAPA HiFi HotStart ReadyMix(2X) (KAPA BIOSYSTEMS) with a cycle consisting of (i) 95′C for 2 minutes, (ii) 98° C. for 20 seconds, (iii) 65° C. for 15 seconds, and (iv) 72° C. for 15 seconds, and a cycle of steps (ii), (iii), and (iv) was repeated 25 times. With the use of NEBuilder HiFi DNA Assembly Master Mix (New England Biolabs), the amplified DNA fragment was ligated to pcDNA3.4 (Thermo Fisher Scientific) cleaved with XbaI (New England Biolabs) and AgeI (New England Biolabs) in accordance with the instructions of the kit. E. coli DH5a (Toyobo Co. Ltd.) cells were transformed using the ligated cyclic plasmid and cultured on an LB agar medium containing 100 μg/ml carbenicillin at 37° C. overnight.

The appeared colonies were further cultured in an LB liquid medium containing 100 sg/ml carbenicillin at 37° C. overnight. Plasmids were purified from the cultured E. coli cells using FastGene Plasmid Mini Kit (Nippon Genetics Co., Ltd.), nucleotide sequences were determined, and plasmids in which target nucleotide sequence insertion was observed were obtained as Ligand 023 and Ligand 024. Nucleotide sequence analysis was commissioned to Macrogen Japan.

Table 4 shows the names of plasmids to express various FAM150B truncated forms, terminal amino acids of FAM150B truncated forms, primers (Fw and Rev) used for first-phase PCR when preparing plasmids to express FAM150B truncated forms, and templates.

TABLE 4

| | Terminal amino acid | | First-phase PCR | | |
|---|---|---|---|---|---|
| Ligand | N | C | Fw primer | Rev primer | Template |
| Ligand 005 | A67 | Q152 | SEQ ID NO: 64 | SEQ ID NO: 60 | Ligand 004 |
| Ligand 006 | G69 | Q152 | SEQ ID NO: 65 | SEQ ID NO: 60 | Ligand 004 |
| Ligand 007 | A73 | Q152 | SEQ ID NO: 66 | SEQ ID NO: 60 | Ligand 004 |
| Ligand 008 | G75 | Q152 | SEQ ID NO: 67 | SEQ ID NO: 60 | Ligand 004 |
| Ligand 009 | G77 | Q152 | SEQ ID NO: 68 | SEQ ID NO: 60 | Ligand 004 |
| Ligand 010 | S79 | Q152 | SEQ ID NO: 69 | SEQ ID NO: 60 | Ligand 004 |
| Ligand 011 | E81 | Q152 | SEQ ID NO: 70 | SEQ ID NO: 60 | Ligand 004 |
| Ligand 012 | R83 | Q152 | SEQ ID NO: 71 | SEQ ID NO: 60 | Ligand 004 |
| Ligand 013 | E85 | Q152 | SEQ ID NO: 72 | SEQ ID NO: 60 | Ligand 004 |
| Ligand 014 | V87 | Q152 | SEQ ID NO: 73 | SEQ ID NO: 60 | Ligand 004 |
| Ligand 015 | R89 | Q152 | SEQ ID NO: 74 | SEQ ID NO: 60 | Ligand 004 |
| Ligand 016 | L91 | Q152 | SEQ ID NO: 75 | SEQ ID NO: 60 | Ligand 004 |
| Ligand 017 | M93 | Q152 | SEQ ID NO: 76 | SEQ ID NO: 60 | Ligand 004 |
| Ligand 018 | D95 | Q152 | SEQ ID NO: 77 | SEQ ID NO: 60 | Ligand 004 |
| Ligand 019 | F97 | Q152 | SEQ ID NO: 78 | SEQ ID NO: 60 | Ligand 004 |
| Ligand 020 | K99 | Q152 | SEQ ID NO: 79 | SEQ ID NO: 60 | Ligand 004 |
| Ligand 021 | L101 | Q152 | SEQ ID NO: 80 | SEQ ID NO: 60 | Ligand 004 |
| Ligand 022 | G103 | Q152 | SEQ ID NO: 81 | SEQ ID NO: 60 | Ligand 004 |
| Ligand 023 | A71 | D150 | SEQ ID NO: 59 | SEQ ID NO: 82 | Ligand 002 |
| Ligand 024 | A71 | M148 | SEQ ID NO: 59 | SEQ ID NO: 83 | Ligand 002 |

SEQ ID NO: 85 shows the amino acid sequence of Ligand 005P protein containing amino acids 67 to 152 of FAM150B encoded by Ligand 005, and SEQ ID NO: 86 shows the DNA sequence encoding the same.

SEQ ID NO: 87 shows the amino acid sequence of Ligand 006P protein containing amino acids 69 to 152 of FAM150B encoded by Ligand 006, and SEQ ID NO: 88 shows the DNA sequence encoding the same.

SEQ ID NO: 89 shows the amino acid sequence of Ligand 007P protein containing amino acids 73 to 152 of FAM150B encoded by Ligand 007, and SEQ ID NO: 90 shows the DNA sequence encoding the same.

SEQ ID NO: 91 shows the amino acid sequence of Ligand 008P protein containing amino acids 75 to 152 of FAM150B encoded by Ligand 008, and SEQ ID NO: 92 shows the DNA sequence encoding the same.

SEQ ID NO: 93 shows the amino acid sequence of Ligand 009P protein containing amino acids 77 to 152 of FAM150B encoded by Ligand 009, and SEQ ID NO: 94 shows the DNA sequence encoding the same.

SEQ ID NO: 95 shows the amino acid sequence of Ligand 010P protein containing amino acids 79 to 152 of FAM150B encoded by Ligand 010, and SEQ ID NO: 96 shows the DNA sequence encoding the same.

SEQ ID NO: 97 shows the amino acid sequence of Ligand 011P protein containing amino acids 81 to 152 of FAM150B encoded by Ligand 011, and SEQ ID NO: 98 shows the DNA sequence encoding the same.

SEQ ID NO: 99 shows the amino acid sequence of Ligand 012P protein containing amino acids 83 to 152 of FAM150B encoded by Ligand 012, and SEQ ID NO: 100 shows the DNA sequence encoding the same.

SEQ ID NO: 101 shows the amino acid sequence of Ligand 013P protein containing amino acids 85 to 152 of FAM150B encoded by Ligand 013, and SEQ ID NO: 102 shows the DNA sequence encoding the same.

SEQ ID NO: 103 shows the amino acid sequence of Ligand 014P protein containing amino acids 87 to 152 of FAM150B encoded by Ligand 014, and SEQ ID NO: 104 shows the DNA sequence encoding the same.

SEQ ID NO: 105 shows the amino acid sequence of Ligand 015P protein containing amino acids 89 to 152 of FAM150B encoded by Ligand 015, and SEQ ID NO: 106 shows the DNA sequence encoding the same.

SEQ ID NO: 107 shows the amino acid sequence of Ligand 016P protein containing amino acids 91 to 152 of FAM150B encoded by Ligand 016 and SEQ ID NO: 108 shows the DNA sequence encoding the same.

SEQ ID NO: 109 shows the amino acid sequence of Ligand 017P protein containing amino acids 93 to 152 of FAM150B encoded by Ligand 017, and SEQ ID NO: 110 shows the DNA sequence encoding the same.

SEQ ID NO: 111 shows the amino acid sequence of Ligand 018P protein containing amino acids 95 to 152 of FAM150B encoded by Ligand 018, and SEQ ID NO: 112 shows the DNA sequence encoding the same.

SEQ ID NO: 113 shows the amino acid sequence of Ligand 019P protein containing amino acids 97 to 152 of FAM150B encoded by Ligand 019, and SEQ ID NO: 114 shows the DNA sequence encoding the same.

SEQ ID NO: 115 shows the amino acid sequence of Ligand 020P protein containing amino acids 99 to 152 of FAM150B encoded by Ligand 020, and SEQ ID NO: 116 shows the DNA sequence encoding the same.

SEQ ID NO: 117 shows the amino acid sequence of Ligand 021P protein containing amino acids 101 to 152 of FAM150B encoded by Ligand 021, and SEQ ID NO: 118 shows the DNA sequence encoding the same.

SEQ ID NO: 119 shows the amino acid sequence of Ligand 022P protein containing amino acids 103 to 152 of FAM150B encoded by Ligand 022, and SEQ ID NO: 120 shows the DNA sequence encoding the same.

SEQ ID NO: 121 shows the amino acid sequence of Ligand 023P protein containing amino acids 71 to 150 of FAM150B encoded by Ligand 023, and SEQ ID NO: 122 shows the DNA sequence encoding the same.

SEQ ID NO: 123 shows the amino acid sequence of Ligand 024P protein containing amino acids 71 to 148 of FAM150B encoded by Ligand 024, and SEQ ID NO: 124 shows the DNA sequence encoding the same.

Constitutions of proteins prepared in this example are schematically shown below and in FIG. 19. Numerical values in parentheses indicate amino acid positions.

### Ligand 004P:

Ig Kappa light chain secretory signal-hexahistidine tag-SUMOstar-HRV3C protease cleavage sequence-FAM150B (25-152)

### Ligand 005P:

Ig Kappa light chain secretory signal-hexahistidine tag-SUMOstar-HRV3C protease cleavage sequence-FAM150B (67-152)

### Ligand 006P:

Ig Kappa light chain secretory signal-hexahistidine tag-SUMOstar-HRV3C protease cleavage sequence-FAM150B (69-152)

### Ligand 007P

Ig Kappa light chain secretory signal-hexahistidine tag-SUMOstar-HRV3C protease cleavage sequence-FAM150B (73-152)

### Ligand 008P:

Ig Kappa light chain secretory signal-hexahistidine tag-SUMOstar-HRV3C protease cleavage sequence-FAM150B (75-152)

### Ligand 009P:

Ig Kappa light chain secretory signal-hexahistidine tag-SUMOstar-HRV3C protease cleavage sequence-FAM150B (77-152)

### Ligand 010P:

Ig Kappa light chain secretory signal-hexahistidine tag-SUMOstar-HRV3C protease cleavage sequence-FAM150B (79-152)

### Li 011P:

Ig Kappa light chain secretory signal-hexahistidine tag-SUMOstar-HRV3C protease cleavage sequence-FAM150B (81-152)

### Ligand 012P:

Ig Kappa light chain secretory signal-hexahistidine tag-SUMOstar-HRV3C protease cleavage sequence-FAM150B (83-152)

### Ligand 013P:

Ig Kappa light chain secretory signal-hexahistidine tag-SUMOstar-HRV3C protease cleavage sequence-FAM150B (85-152)

### Ligand 014P:

Ig Kappa light chain secretory signal-hexahistidine tag-SUMOstar-HRV3C protease cleavage sequence-FAM150B (87-152)

### Ligand 015P:

Ig Kappa light chain secretory signal-hexahistidine tag-SUMOstar-HRV3C protease cleavage sequence-FAM150B (89-152)

### Ligand 016P:

Ig Kappa light chain secretory signal-hexahistidine tag-SUMOstar-HRV3C protease cleavage sequence-FAM150B (91-152)

### Ligand 017P:

Ig Kappa light chain secretory signal-hexahistidine tag-SUMOstar-HRV3C protease cleavage sequence-FAM150B (93-152)

### Ligand 18P:

Ig Kappa light chain secretory signal-hexahistidine tag-SUMOstar-HRV3C protease cleavage sequence-FAM150B (95-152)

### Ligand 019P:

Ig Kappa light chain secretory signal-hexahistidine tag-SUMOstar-HRV3C protease cleavage sequence-FAM150B (97-152)

### Ligand 020P:

Ig Kappa light chain secretory signal-hexahistidine tag-SUMOstar-HRV3C protease cleavage sequence-FAM150B (99-152)

### Ligand 021P:

Ig Kappa light chain secretory signal-hexahistidine tag-SUMOstar-HRV3C protease cleavage sequence-FAM150B (101-152)

### Ligand 022P:

Ig Kappa light chain secretory signal-hexahistidine tag-SUMOstar-HRV3C protease cleavage sequence-FAM150B (103-152)

### Ligand 023P:

Ig Kappa light chain secretory signal-hexahistidine tag-SUMOstar-HRV3C protease cleavage sequence-FAM150B (71-150)

### Ligand 024P:

Ig Kappa light chain secretory signal-hexahistidine tag-SUMOstar-HRV3C protease cleavage sequence-FAM150B (71-148)

### Example 11

### Measurement of Binding Activity to ALK 001P and LTK 001P by Surface Plasmon Resonance Analysis

### Expression of FAM150B Truncated Form

Various FAM150B truncated forms were expressed using Ligand 003 and Ligand 005 to Ligand 024 plasmids and Expi293 Expression System (Thermo Fisher Scientific). Expi293 cells cultured in Expi293 Expression Medium were diluted to 2.9×10⁶ cells/ml with Expression Medium, the resulting culture solution (25.5 ml) was introduced into a 125-ml flask, and 30 sg each of Ligand 003 and Ligand 005 to Ligand 024 were transfected into the Expi293 cells in accordance with the instructions.

18 to 20 hours after transfection. Enhancers 1 and 2 included in the kit were added in amounts of 0.15 ml and 1.5 ml, respectively, and culture was continued. Culture was carried out in the presence of 8% CO₂ at 125 rpm and 37° C. The culture supernatant was collected via centrifugation 4 days after transfection. The collected supernatant was cryopreserved at −30° C. to −80° C. before purification.

### Purification of FAM150B Truncated Form

After the culture supernatant was thawed, 1.5 ml of 1 M Tris-HCl (pH 8.0) and 2 ml of a 50% suspension of Ni Sepharose 6 FastFlow (GE Healthcare Japan) in DW were added, the mixture was stirred at 4° C. for 10 minutes, and Ligand 003P and Ligand 005P to Ligand 024P were allowed to adsorb to resin. Thereafter, the suspension was transferred to an empty chromatography column, the resin was washed with 50 mM Tris-HCl (pH 8.0), 500 mM NaCl, and 20 mM imidazole, and Ligand 003P and Ligand 005P to Ligand 024P were eluted with the aid of 50 mM Tris-HCl (pH 8.0), 500 mM NaCl, and 500 mM imidazole.

The resulting eluate was subjected to separation using Superdex 75 10/300 (GE Healthcare Japan) connected to AKTA explorer (GE Healthcare Japan) at a flow rate of 0.7 ml/min, and the main peak fraction was collected and used as a sample for SPR-based interaction analysis.

### Verification of Binding of ALK 001P or LTK 001P to FAM150B Truncated Form

A region of amino acids 71 to 152 of FAM150B was found to bind to ALK 001P and LTK 001P. Thus, various FAM150B truncated forms were examined using Biacore 8K instrument (GE Healthcare Japan) by the surface plasmon resonance (SPR) technique to identify border amino acid residue that would exhibit changes in intensity to bind to ALK 001P and LTK 001P.

### Immobilization of ALK 001P and LTK 001P on Sensor Chip

ALK 001P contained at 7.5 μg/ml (calculated based on the molecular weight without sugar chain) and LTK 001P contained at 10 μg/ml (calculated based on the molecular weight without sugar chain) in HBS-P+ were introduced at a flow rate of 10 μl/min for 60 seconds and immobilized on the surface coated with Protein A of Series S Sensor Chip Protein A (GE Healthcare Japan). The amount of ALK001P immobilized was 590 to 670 RU, and the amount of LTK 001P immobilized was 520 to 640 RU.

### Acquisition of Bond Dissociation Curve

Six concentrations of Ligand 003P and Ligand 005P to Ligand 024P at 2-fold dilution with HBS-P+ each from 20 nM were prepared. Ligand 003P and Ligand 005P to Ligand 024P at various concentrations and HBS-P+(concentration 0) were introduced on the surface of the immobilized ALK 001P or LTK 001P and on the untreated surface coated with Protein A (baseline) at a flow rate of 30 μl/min for 360 seconds, and the binding curves were recorded.

Subsequently, HBS-P+ was introduced on the surface of the immobilized ALK 001P or LTK 001P and on the untreated surface coated with Protein A (baseline) at a flow rate of 30 μl/min for 600 seconds, and the dissociation curves were recorded.

ALK 001P and LTK 001P were dissociated from Protein A coating the sensor chip surface (i.e., regeneration) by introduction of 10 mM Glycine-HCl(pH 1.6) at a flow rate of 30 μl/min for 30 seconds. The bond dissociation curve modified by subtracting the baseline was prepared using Biacore8K Evaluation Software (GE Healthcare Japan).

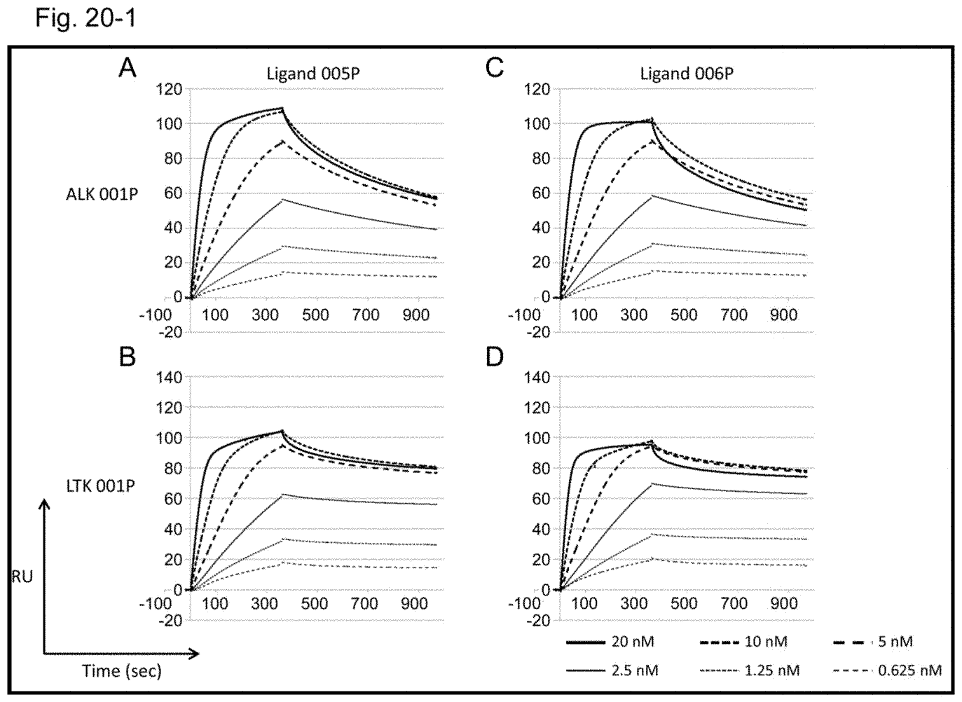

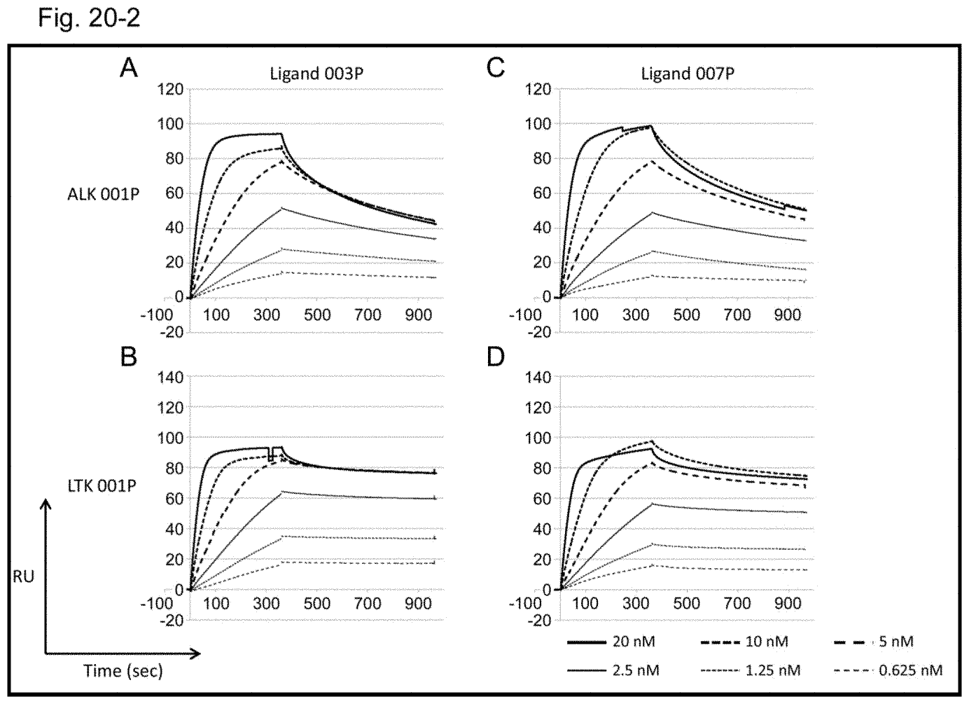

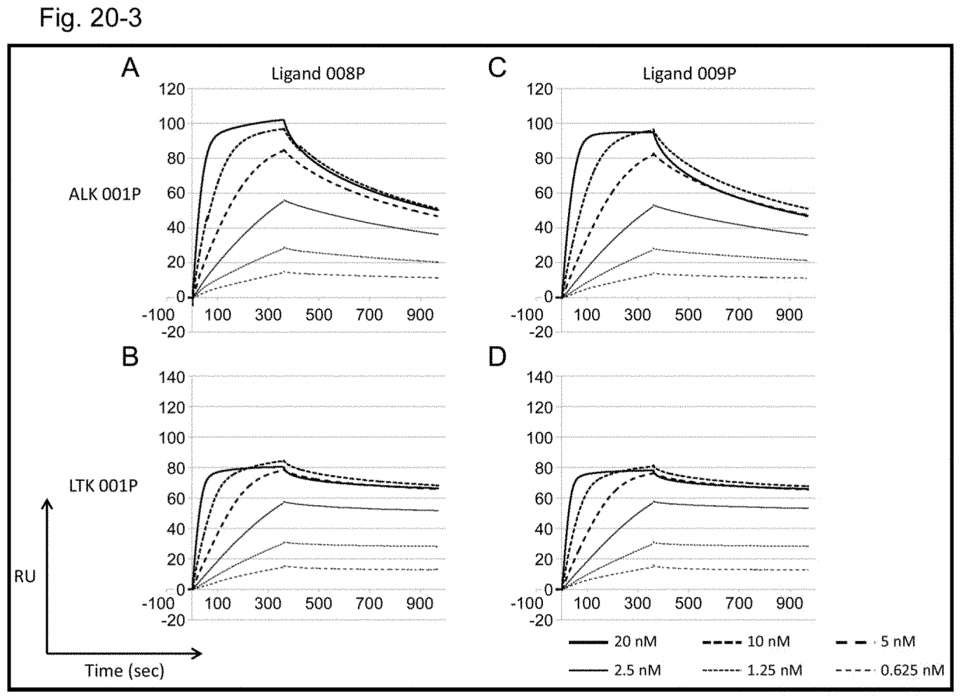

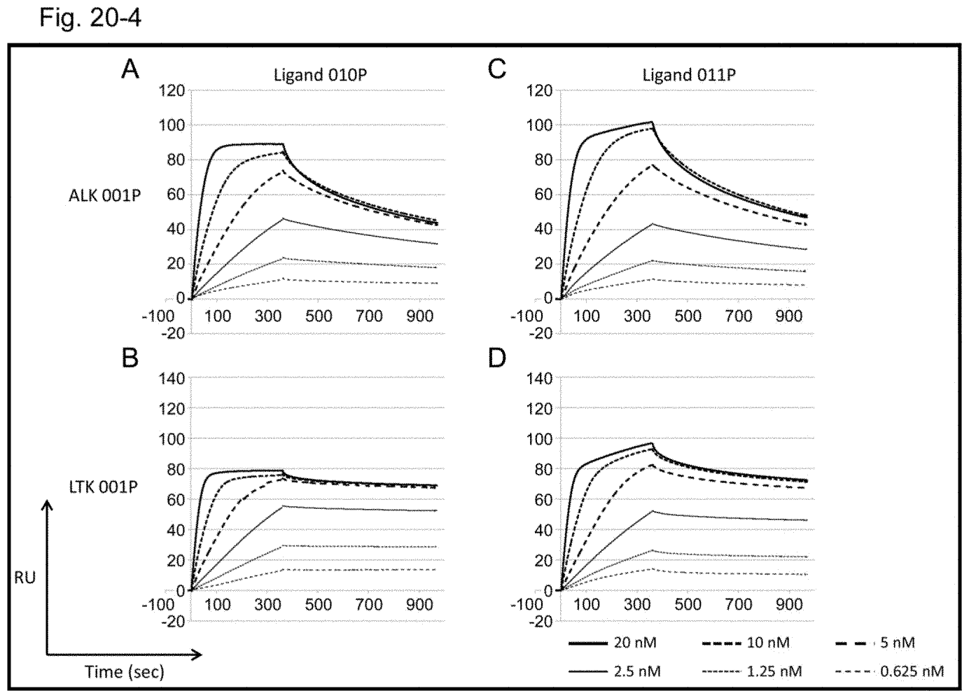

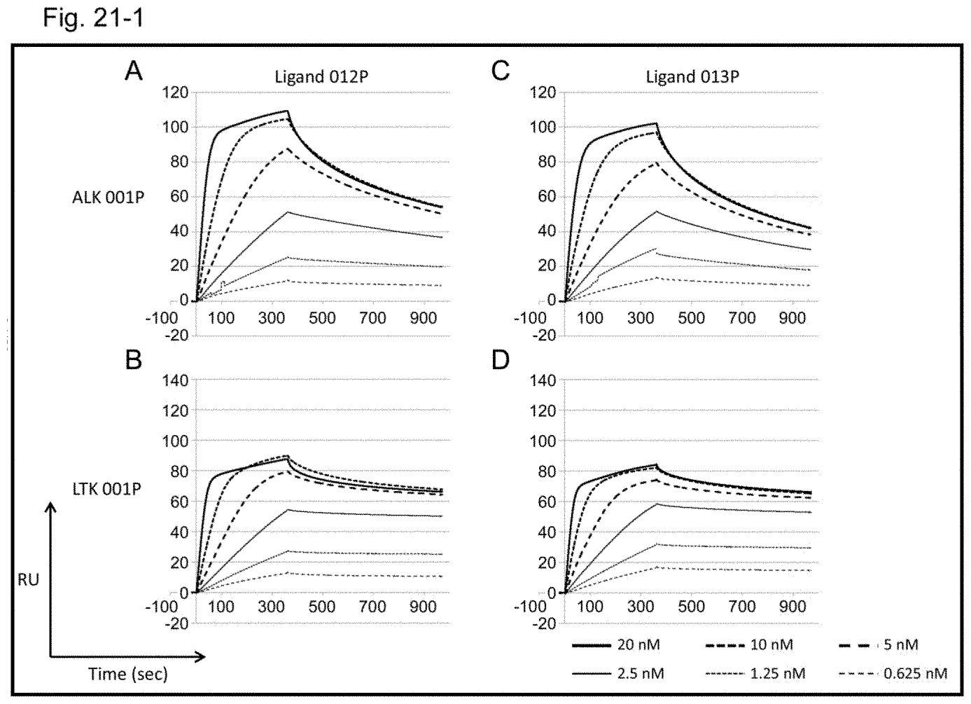

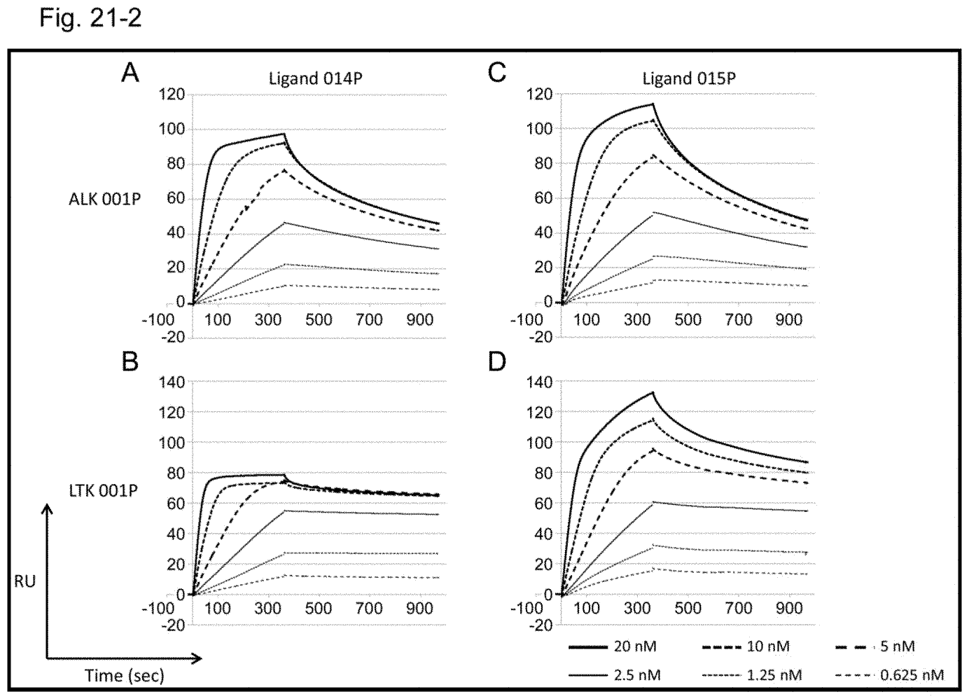

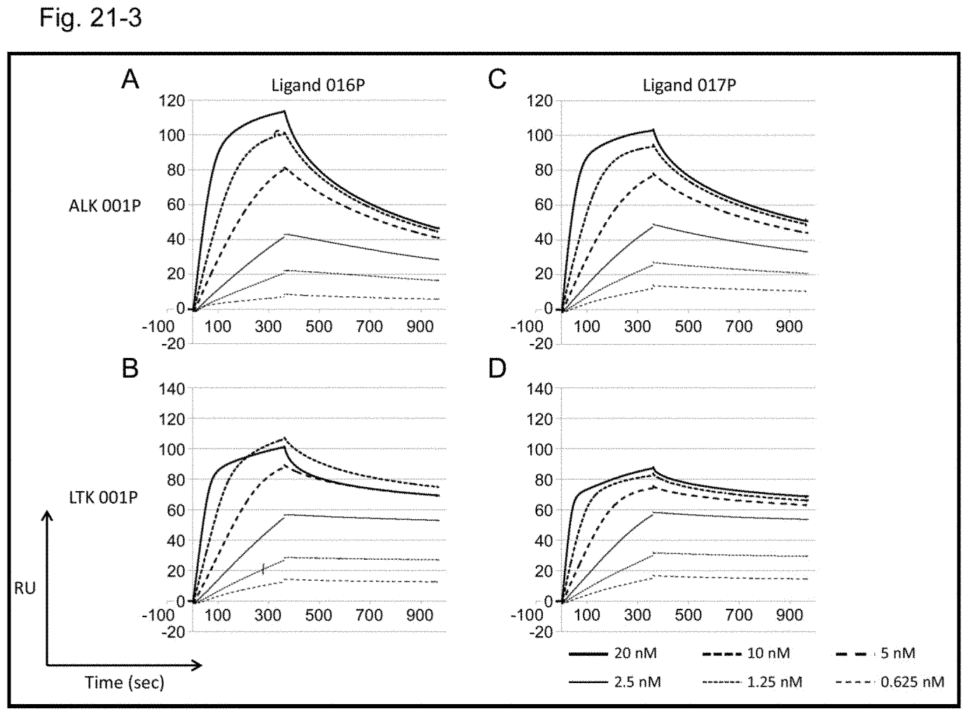

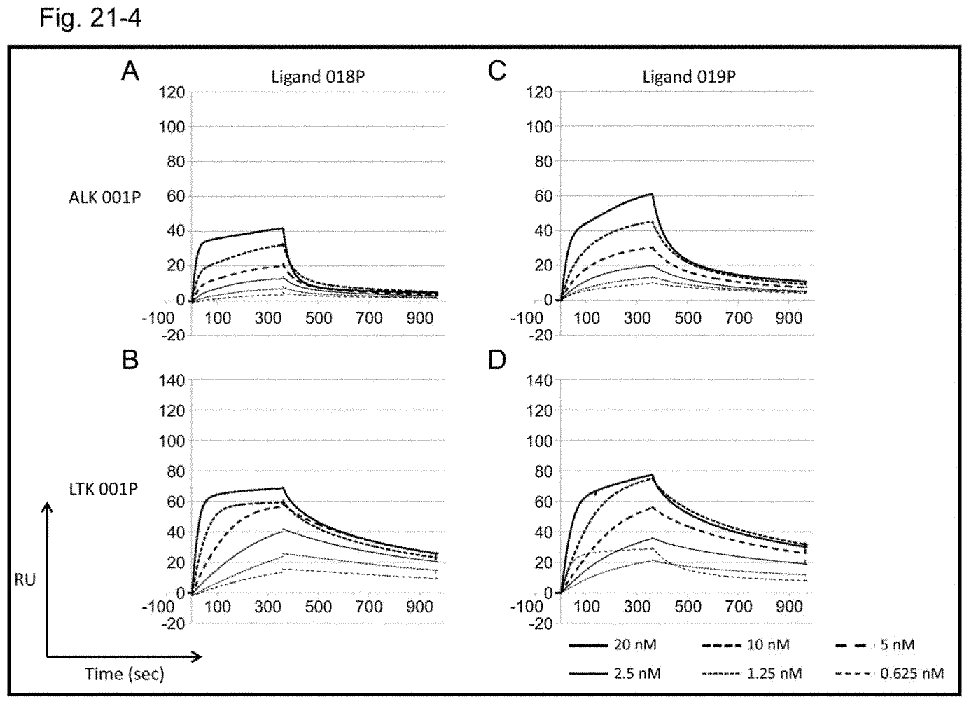

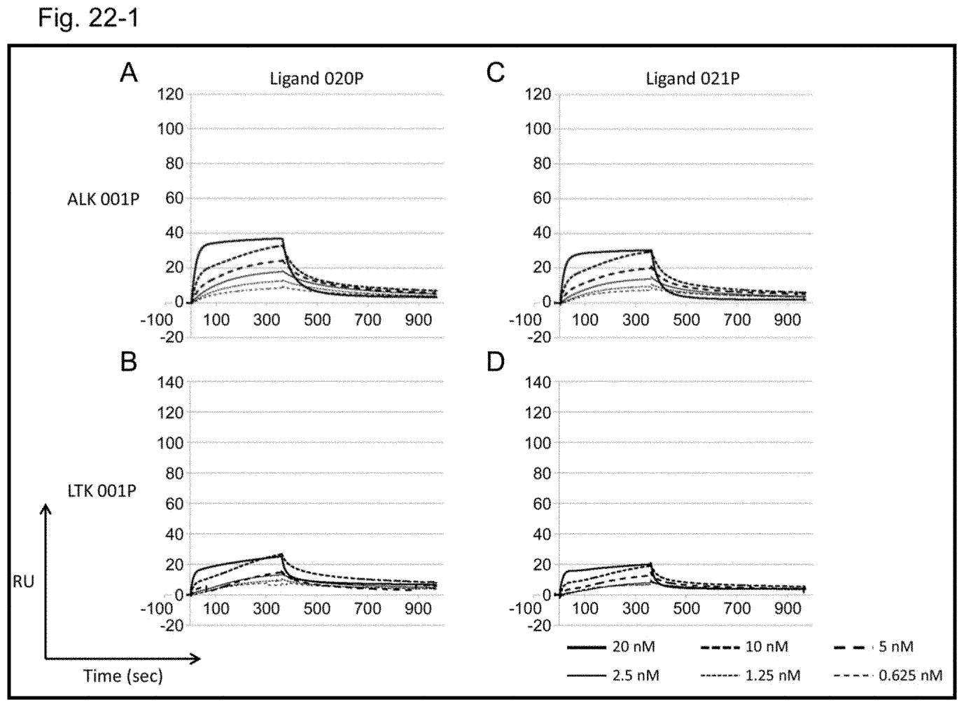

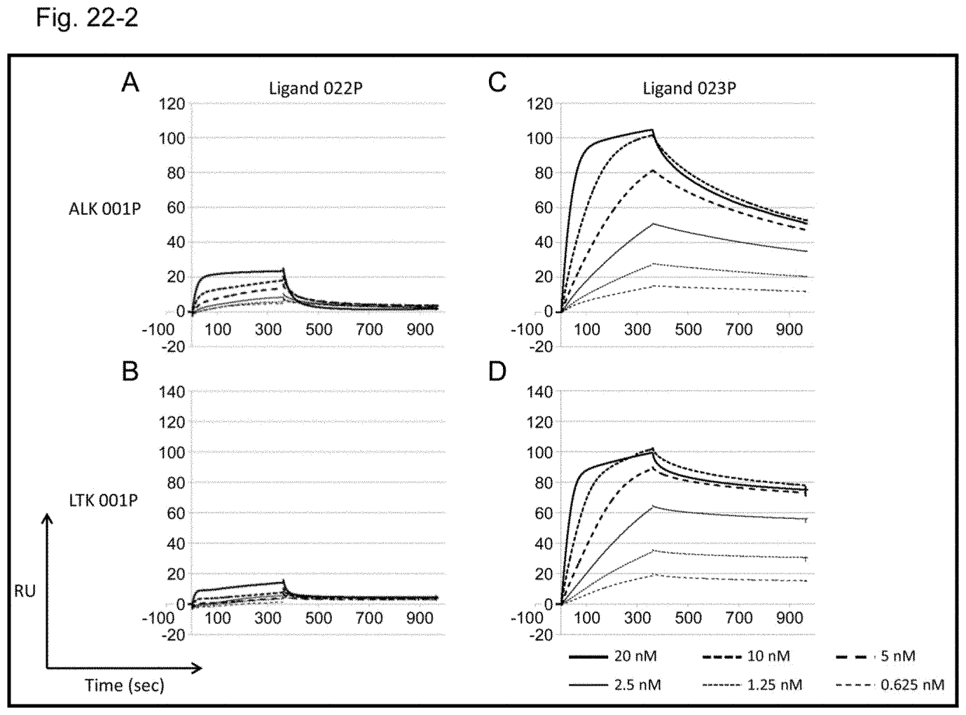

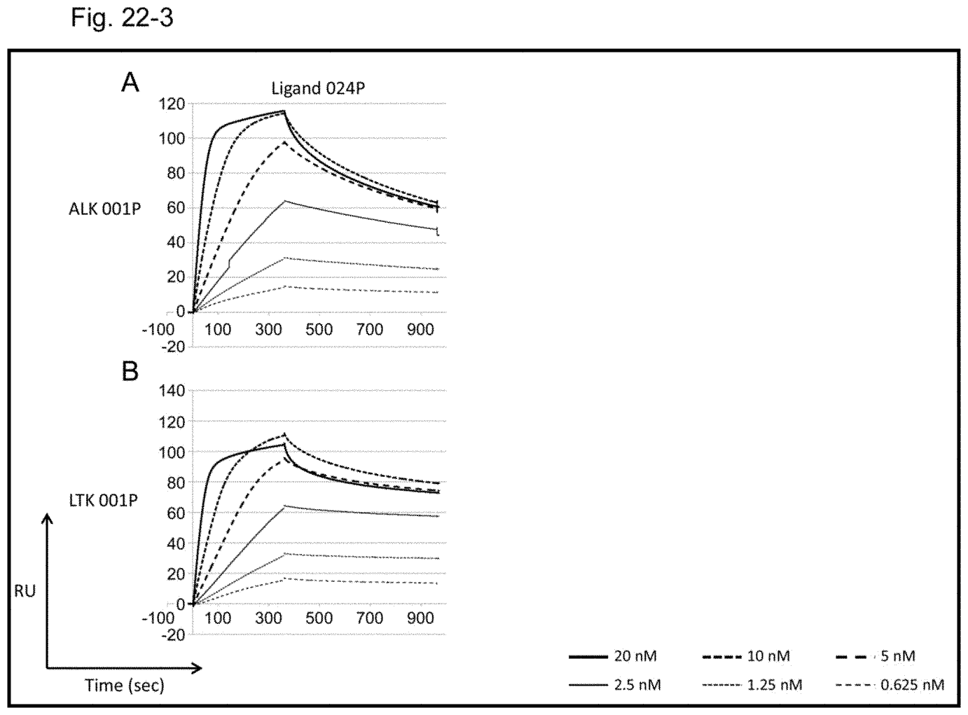

FIGS. 20-1 to 20-4, FIGS. 21-1 to 21-4, and FIGS. 22-1 to 22-3 each show the results of verification of binding of ALK001P and LTK 001P to Ligand 003P and Ligand 005P to Ligand 024P via surface plasmon resonance analysis. The results demonstrate that Ligand 003P, Ligand 005P to Ligand 017P, Ligand 023P, and Ligand 024P sufficiently bind to both ALK 001P and LTK 001P and that they are suitable as CAR target binding domains targeting ALK and LTK. In contrast, Ligand 018P to Ligand 022P were dissociated from ALK and LTK significantly faster than other ligands and exhibited lowered binding intensity.

### Example 12

### Preparation of 4-1BB-Type CAR-Expressing Plasmid

In this example, CAR 009 and CAR 010 plasmids were prepared by replacing the CD28 domain of CAR 002 and 003 with 4-1BB.

### Artificial Synthesis of CD8α+4-1BB Gene

On the basis of the amino acid sequence information of human 4-1BB (SEQ ID NO: 125) described in WO 2015/069922, a DNA sequence (SEQ ID NO: 126) comprising an upstream 15-base region and a downstream 15-base region of the CD28 region of CAR002 and CAR 003 added to the DNA sequence codon-optimized for human was designed, artificially synthesized, and then inserted into a pEX-K4J1 vector (synthesis was commissioned to Eurofins Genomics K.K.).

### Amplification of CD8α+4-1BB by PCR

PCR amplification was conducted using the artificially synthesized gene (SEQ ID NO: 126) as a template. Primers used for PCR are represented by SEQ ID NO: 127 and SEQ ID NO: 128. PCR was performed using PrimeSTAR Max DNA polymerase (Takara Bio Inc.) with a cycle consisting of 98° C. for 10 seconds and 68° C. for 30 seconds, which was repeated 35 times. After the PCR reaction, an aliquot of the sample was separated via 2% agarose gel electrophoresis, the amplification product of the size of interest was confirmed, and the remaining sample after PCR was treated with DpnI in accordance with the instructions of the KOD-Plus-Mutagenesis Kit (Toyobo Co. Ltd.) to remove the template plasmid.

### Amplification of Linear Fragment of CAR 002 or CAR 003 by Inverse-PCR

In order to delete the CD28 region from CAR 002 or CAR 003, a linear fragment was prepared by inverse-PCR. As primers used for inverse-PCR, primers represented by SEQ ID NO: 129 and SEQ ID NO: 130 were designed and synthesized (synthesis was commissioned to Eurofins Genomics K.K.). CAR 002 or CAR 003 was adjusted to 50 ng/μl and used as a template, and the concentration of each PCR primer was adjusted to 0.2 μM in the reaction solution. Inverse-PCR was performed using KOD-Plus-Mutagenesis Kit (Toyobo Co. Ltd.) with the reaction composition designated in the instructions of the kit and with a cycle consisting of (i) 94° C. for 2 minutes, (ii) 98° C. for 10 seconds, and (iii 68°) C for 7 minutes, and a cycle of steps (ii) and (iii) was repeated 10 times. After the PCR reaction, an aliquot of the sample was separated via 1% agarose gel electrophoresis, amplification of a linear fragment of the size of interest was confirmed, the remaining sample after PCR was treated with DpnI in accordance with the instructions of the KOD-Plus-Mutagenesis Kit (Toyobo Co. Ltd.), and methylated template plasmid CAR 002 or CAR 003 was cleaved and removed.

### Ligation of CAR 002 or CAR 003 Linear Fragment to CD8a. +4-1BB Amplified Fragment

The CAR 002 or CAR 003 linear fragment was ligated to the CD8α+4-1BB amplified fragment using NEBuilder HiFi DNA Assembly Master Mix (New England Biolabs). After the reaction at 50° C. for 15 minutes, E. coli DH5a (Toyobo Co. Ltd.) cells were transformed using the ligated cyclic plasmid and then cultured on an LB agar medium containing 100 μg/ml carbenicillin for about 16 hours.

The appeared colonies were further cultured in an LB liquid medium containing 100 μg/ml carbenicillin for about 16 hours. Plasmids were purified from the cultured E. coli cells using QIAprep Spin Miniprep Kit (Qiagen), nucleotide sequences were determined, and plasmids in which target nucleotide sequence insertion was observed were obtained as a FAM150A 4-1BB-type plasmid (CAR 009; FAM150A-8αBBz) and a FAM150B 4-1BB-type plasmid (CAR 010; FAM150B-8αBBz). Nucleotide sequence analysis was commissioned to Eurofins Genomics K.K.

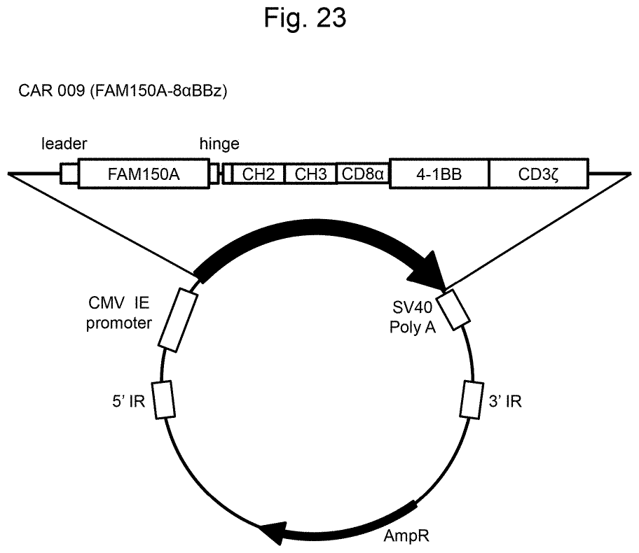

FIG. 23 shows a vector map of CAR 009 (FAM150A-8a28z), and the DNA sequence of a translation region of CAR 009 is represented by SEQ ID NO: 131.

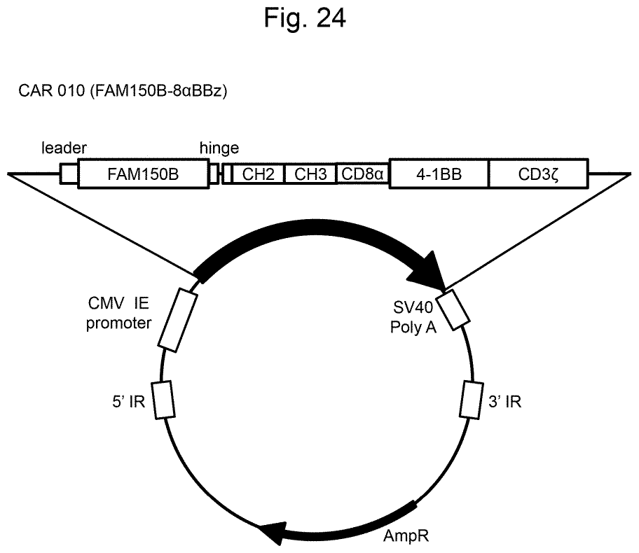

FIG. 24 shows a vector map of CAR 010 (FAM150B-8a28z), and the DNA sequence of a translation region of CAR 010 is represented by SEQ ID NO: 132.

### Preparation of FAM150A Truncated 4-1BB-Type CAR-Expressing Plasmid (CAR 011; FAM150ATr-8αBBz) and FAM150B Truncated 4-1BB-Type CAR-Expressing Plasmid (CAR 012; FAM150BTr-8αBBz)

With the use of CAR 009 or CAR 010 prepared above as a template, a FAM150A truncated 4-1BB-type CAR-expressing plasmid (CAR 011: FAM150ATr-8αBBz) and a FAM150B truncated 4-1BB-type CAR-expressing plasmid (CAR 012; FAM150BTr-8αBBz) were prepared by inverse-PCR. The primers for CAR 002 linearization (SEQ ID NO: 52 and SEQ ID NO: 53) and the primers for CAR 003 linearization (SEQ ID NO: 54 and SEQ ID NO: 55) described above were used for inverse-PCR.

CAR 009 or CAR 010 was adjusted to 50 ng/μl and used as a template, and the concentration of each PCR primer was adjusted to 0.2 μM in the reaction solution. Inverse-PCR was performed using KOD-Plus-Mutagenesis Kit (Toyobo Co. Ltd.) with the reaction composition designated in the instructions of the kit and with a cycle consisting of (i) 94° C. for 2 minutes, (ii) 98° C. for 10 seconds, and (iii) 68° C. for 7 minutes, and a cycle of steps (ii) and (iii) was repeated 10 times.

After the PCR reaction, an aliquot of the sample was separated via 1% agarose gel electrophoresis, amplification of the linear plasmid of the size of interest was confirmed, the remaining sample after PCR was treated with DpnI in accordance with the instructions of the KOD-Plus-Mutagenesis Kit (Toyobo Co. Ltd.), and methylated template plasmid CAR 009 or CAR 010 was cleaved and removed. Thereafter, the linear plasmid was phosphorylated with T4 Polynucleotide Kinase included in the kit and self-ligated to form a cyclic plasmid. Thereafter, E. coli DH5a. (Toyobo Co. Ltd.) cells were transformed using the cyclic plasmid and then cultured on an LB agar medium containing 100 μg/ml carbenicillin for about 16 hours.

The appeared colonies were further cultured in an LB liquid medium containing 100 μg/ml carbenicillin for about 16 hours. Plasmids were purified from the cultured E. coli cells using QIAprep Spin Miniprep Kit (Qiagen), nucleotide sequences were determined, and plasmids in which target nucleotide sequence deletion was observed were obtained as a FAM150A truncated 4-1BB-type CAR-expressing plasmid (CAR 011; FAM150ATr-8αBBz) and a FAM150B truncated 4-1BB-type CAR-expressing plasmid (CAR 012; FAM150BTr-8αBBz).

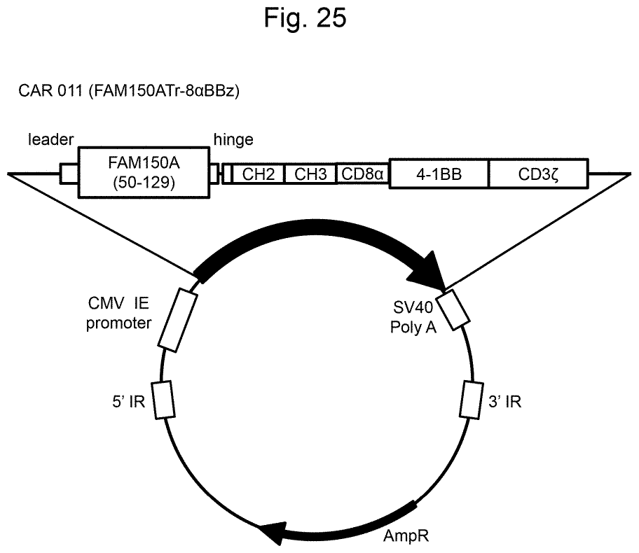

FIG. 25 shows a vector map of CAR 011 (FAM150ATr-8αBBz), and the DNA sequence of a translation region of CAR 011 is represented by SEQ ID NO: 133.

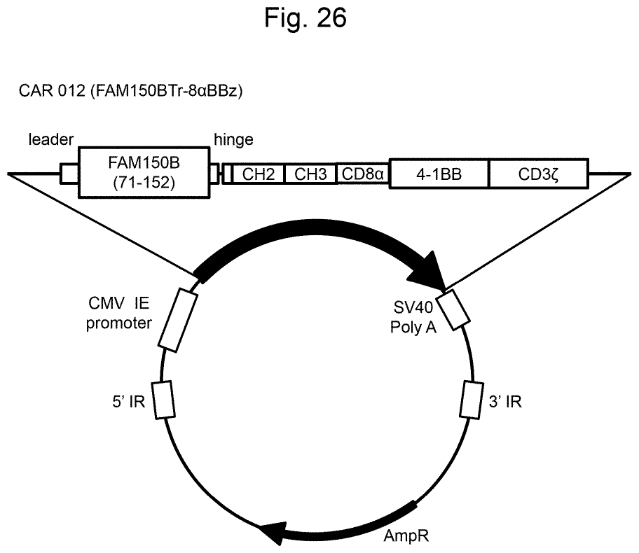

FIG. 26 shows a vector map of CAR 012 (FAM150BTr-8αBBz), and the DNA sequence of a translation region of CAR 012 is represented by SEQ ID NO: 134.

Preparation of Humanized ALK48 scFv 4-1BB-Type (CAR 013: hALK48-8αBBz) and Mouse ALK48 scFv 4-1BB-Type CAR-Expressing Plasmids (CAR 014 ALK48-8αBBz)

CAR 004, CAR 005, CAR 009, or CAR 010 (about 1 μg equivalent each) was digested with restriction enzymes XhoI and DraIII (New England Biolabs) at 37° C. for about 2 hours. After the enzyme treatment, the reaction solution was separated via 1% agarose gel electrophoresis, the enzyme-treated CAR 009 or CAR 010 fragment (on the vector side) and the insert fragment cleaved from CAR 004 or CAR 005 (SEQ ID NO: 5 or 6) were removed from the gel, and the fragments were purified using NucleoSpin Gel and PCR Clean-up® (MACHEREY-NAGEL, Takara Bio Inc.). The purified vector fragment was ligated to the insert fragment using DNA ligation kit (Mighty Mix, Takara Bio Inc.). E. coli DH5u (Toyobo Co. Ltd.) cells were transformed using the ligated cyclic plasmid and then cultured on an LB agar medium containing 100 μg/ml carbenicillin for about 16 hours.

The appeared colonies were further cultured in an LB liquid medium containing 100 μg/ml carbenicillin for about 16 hours. Plasmids were purified from the cultured E. coli cells using QIAprep Spin Miniprep Kit (Qiagen), nucleotide sequences were determined, and plasmids in which target nucleotide sequence insertion was observed were obtained as a humanized ALK48 scFv 4-1BB-type (CAR 013; hALK48-8αBBz) and a mouse ALK48 scFv 4-1BB-type CAR-expressing plasmids (CAR 014; ALK48-8αBBz). Nucleotide sequence analysis was commissioned to Eurofins Genomics K.K.

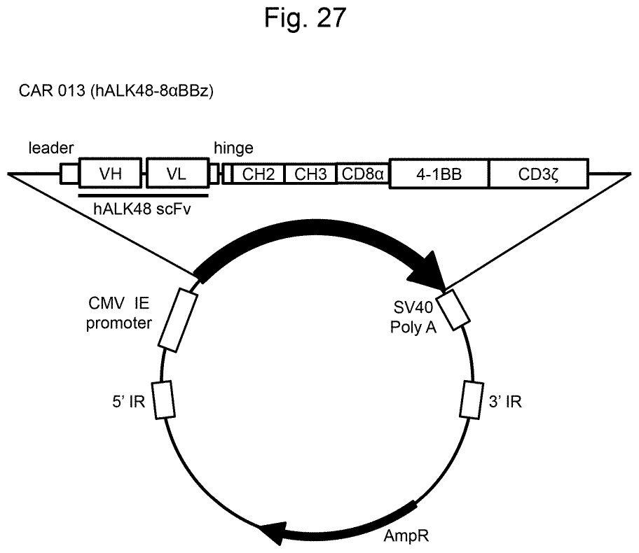

FIG. 27 shows a vector map of CAR 013 (hALK48-8αBBz), and the DNA sequence of a translation region of CAR 013 is represented by SEQ ID NO: 135.

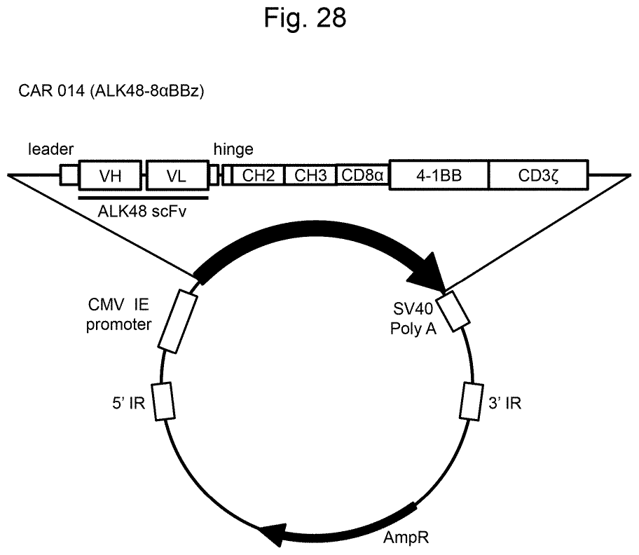

FIG. 28 shows a vector map of CAR 014 (ALK48-8αBBz), and the DNA sequence of a translation region of CAR 014 is represented by SEQ ID NO: 136.

### Example 13

Comparison of Antitumor Activity of FAM150A 4-1BB-Type CAR-T, FAM150B 4-1BB-Type CAR-T, FAM150A Truncated 4-1BB-Type CAR-T, FAM150B Truncated 4-1BB-Type CAR-T, Humanized ALK48 scFv 4-1BB-Type CAR-T, and Mouse ALK48 scFv 4-1BB-Type CAR-T

In accordance with the method of CAR-T cell culture and proliferation in Example 2, CAR-T cells were prepared using the CAR 009- to CAR 014-expressing plasmids prepared in the examples.

CAR-T cells obtained by electrical introduction into PBMCs using CAR 009 to CAR 014 and T cell culture and proliferation are described herein as follows.
CAR-T 009: FAM150A 4-1BB-type CAR-T (FAM150A-8αBBz CAR-T)
CAR-T 010: FAM150B 4-1BB-type CAR-T (FAM150B-8αBBz CAR-T)
CAR-T 011: FAM150A truncated 4-1BB-type CAR-T (FAM150ATr-8αBBz CAR-T)
CAR-T 012: FAM150B truncated 4-1BB-type CAR-T (FAM150BTr-8αBBz CAR-T)
CAR-T 013: Humanized ALK48 scFv 4-1BB-type CAR-T (hALK48-8αBBz CAR-T)
CAR-T 014: Mouse ALK48 scFv 4-1BB-type CAR-T (ALK48-8αBBz CAR-T)

The amino acid sequence of CAR 009 and the nucleotide sequence encoding the same are represented by SEQ ID NO: 137 and SEQ ID NO: 131, respectively.

The amino acid sequence of CAR 010 and the nucleotide sequence encoding the same are represented by SEQ ID NO: 138 and SEQ ID NO: 132, respectively.

The amino acid sequence of CAR 011 and the nucleotide sequence encoding the same are represented by SEQ ID NO: 139 and SEQ ID NO: 133, respectively.

The amino acid sequence of CAR 012 and the nucleotide sequence encoding the same are represented by SEQ ID NO: 140 and SEQ ID NO: 134, respectively.

The amino acid sequence of CAR 013 and the nucleotide sequence encoding the same are represented by SEQ ID NO: 141 and SEQ ID NO: 135, respectively.

The amino acid sequence of CAR 014 and the nucleotide sequence encoding the same are represented by SEQ ID NO: 142 and SEQ ID NO: 136, respectively.

Table 5 shows CAR expression rates in the CAR-T cells obtained by introduction of CAR-expressing plasmids into PBMCs derived from a healthy adult donor (Donor-1) and culture.

TABLE 5

| Donor | Plasmid | CAR-T | Expression rate |
|---|---|---|---|
| Donor-1 | CAR 009 | CAR-T 009 | 29.3 |
| | CAR 010 | CAR-T 010 | 12.3 |
| | CAR 011 | CAR-T 011 | 29.0 |
| | CAR 012 | CAR-T 012 | 6.04 |
| | CAR 013 | CAR-T 013 | 19.7 |
| | CAR 014 | CAR-T 014 | 14.7 |

### Measurement of Antitumor Activity of CAR-T

In order to measure antitumor activity of CAR-T 009 to CAR-T 014 obtained above, co-culture with solid tumor cells was conducted. In this example, passaged cell lines of SH-SY5Y and a breast cancer cell line (MDA-MB231 ffLuc, JCRB Cell Bank) were used as target tumor cells. SH-SY5Y cells and MDA-MB231 ffLuc cells were subjected to passage culture in D-MEM/Ham's F-12 medium containing 15% FBS, 1% penicillin/streptomycin, and 1% non-essential amino acid solution, and co-culture was also performed in a similar medium.

Activity evaluation was performed in accordance with the method of Example 3 using BD Accuri™C6 Plus flow cytometer (BD Biosciences). MDA-MB231 ffLuc cells were analyzed by flow cytometry using 2 μl of PE Anti-Human EGFR antibody (Miltenyi Biotec) instead of PE anti-human Ganglioside GD2 antibody, and the number of EGFR-positive tumor cells was determined based on the number of counting beads.

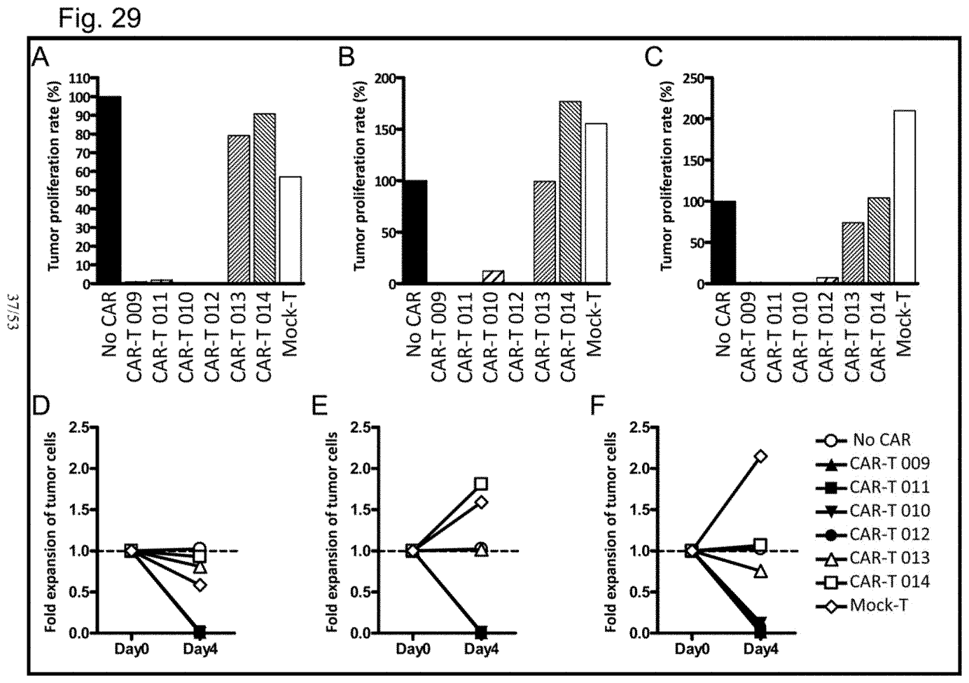

FIG. 29 shows antitumor activity of CAR-T 009 to CAR-T 014 or mock-T cells derived from Donor-1 against SH-SY5Y and the tumor cell proliferation curves. As a result, the effects of CAR-T009, CAR-T 010, CAR-T 011, and CAR-T 012 to kill SH-SY5Y were verified. In contrast, the effects of CAR-T 013 and CAR-T 014 were not significantly different from those of mock-T cells, and substantially no effects were observed.

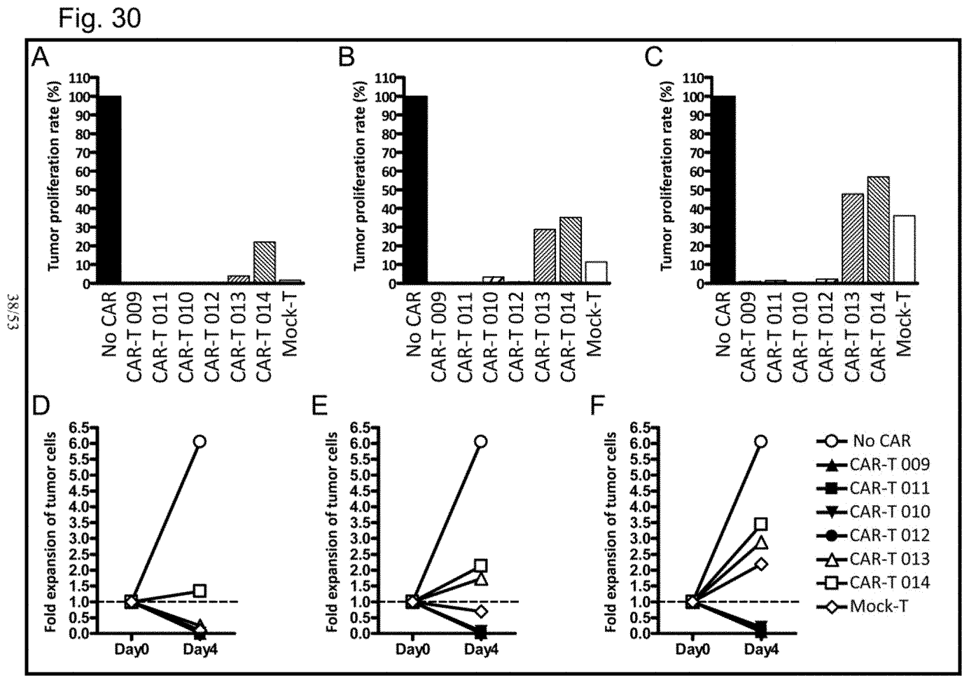

FIG. 30 shows antitumor activity of CAR-T 009 to CAR-T 014 or mock-T cells derived from Donor-1 against MDA-MB231 ffLuc and the tumor cell proliferation curves. As a result, the effects of CAR-T 009, CAR-T 010, CAR-T 011, and CAR-T 012 to kill MDA-MB231 ffLuc were verified. In contrast, the effects of CAR-T 013 and CAR-T 014 were lower than those of mock-T cells, and substantially no effects were observed.

This example indicates that antitumor activity of FAM150A (full-length and truncated) CAR-T and FAM150B (full-length and truncated) CAR-T against neuroblastoma cell line and breast cancer cell line is higher than that of ALK48 scFv-type (humanized and mouse) CAR-T.

### Example 14

### Preparation of FAM150B T14, FAM150B T15, and FAM150B T17 to FAM150B T19 Truncated CAR-Expressing Plasmids

With the use of CAR 008 (FAM150BTr-28z) prepared in Example 7 as a template, plasmids expressing truncated forms CAR 015 to CAR 019 were further prepared. Forward primers for inverse-PCR were designed for CAR 015 amplification (SEQ ID NO: 156), CAR 016 amplification (SEQ ID NO: 157). CAR 017 amplification (SEQ ID NO: 158), CAR 018 amplification (SEQ ID NO: 159), and CAR 019 amplification (SEQ ID NO: 160). The sequence represented by SEQ ID NO: 55 was used as a reverse primer.

CAR 008 was adjusted to 50 ng/μl and used as a template, and the concentration of each PCR primer was adjusted to 0.2 μM in the reaction solution. Inverse-PCR was performed using KOD-Plus-Mutagenesis Kit (Toyobo Co. Ltd.) with the reaction composition designated in the instructions of the kit and with a cycle consisting of (i) 94° C. for 2 minutes, (ii) 98° C. for 10 seconds, and (iii 68°) C for 7 minutes, and a cycle of steps (ii) and (iii) was repeated 10 times.

After the PCR reaction, an aliquot of the sample was separated via 1% agarose gel electrophoresis, amplification of a linear plasmid of the size of interest was confirmed, the remaining sample after PCR was treated with DpnI in accordance with the instructions of the KOD-Plus-Mutagenesis Kit (Toyobo Co. Ltd.), and methylated template plasmid CAR 008 was cleaved and removed. Thereafter, the linear plasmid was phosphorylated with T4 Polynucleotide Kinase included in the kit and self-ligated to form a cyclic plasmid. Thereafter, E. coli DH5a (Toyobo Co. Ltd.) cells were transformed using the cyclic plasmid and then cultured on an LB agar medium containing 100 μg/ml carbenicillin for about 16 hours.

The appeared colonies were further cultured in an LB liquid medium containing 100 μg/ml carbenicillin for about 16 hours. Plasmids were purified from the cultured E. coli cells using QIAprep Spin Miniprep Kit (Qiagen), nucleotide sequences were determined, and plasmids in which target nucleotide sequence deletion was observed were obtained as FAM150B truncated CD28-type CAR-expressing plasmids (CAR 015: FAM150BT14-28z, CAR 016: FAM150BT15-28z, CAR 017: FAM150BT17-28z, CAR 018: FAM150BT18-28z, and CAR 019: FAM150BT19-28z).

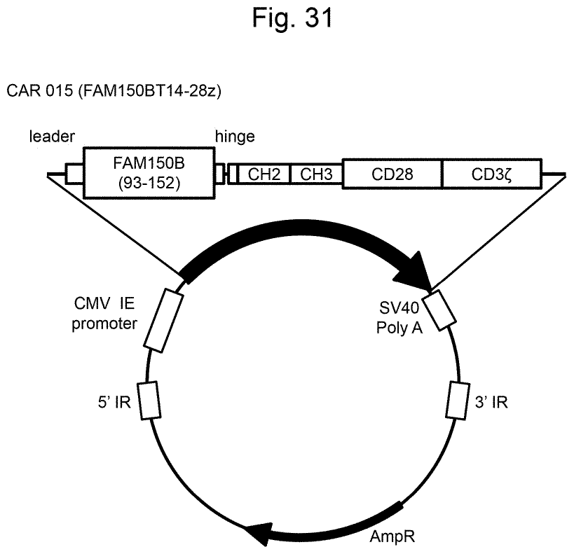

FIG. 31 shows a vector map of CAR 015 (FAM150BT14-28z), and the DNA sequence of a translation region of CAR 015 is represented by SEQ ID NO: 161.

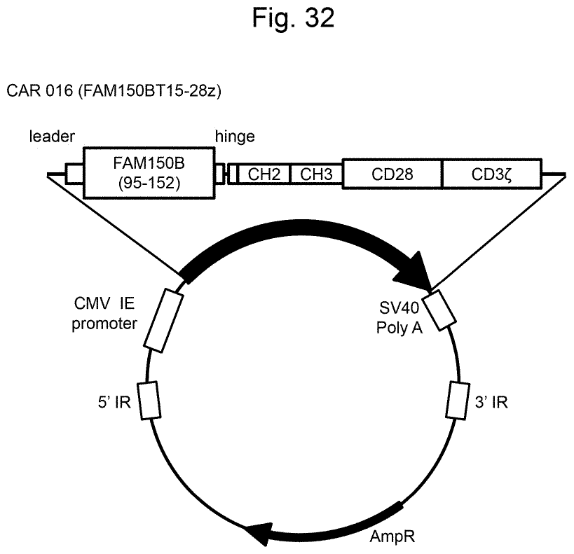

FIG. 32 shows a vector map of CAR 016 (FAM150BT15-28z), and the DNA sequence of a translation region of CAR 016 is represented by SEQ ID NO: 162.

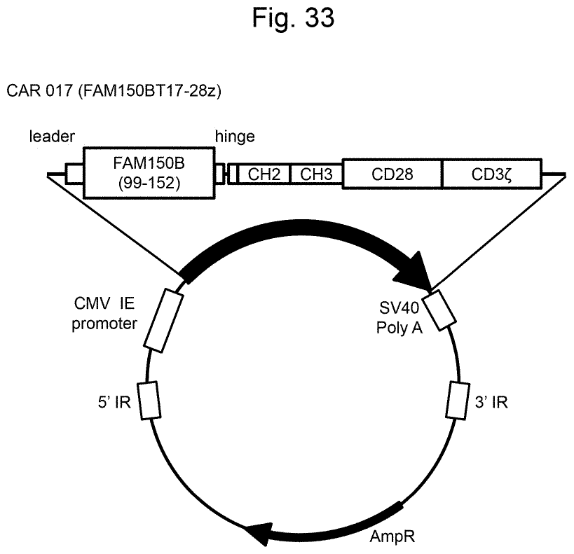

FIG. 33 shows a vector map of CAR 017 (FAM150BT17-28z), and the DNA sequence of a translation region of CAR 017 is represented by SEQ ID NO: 163.

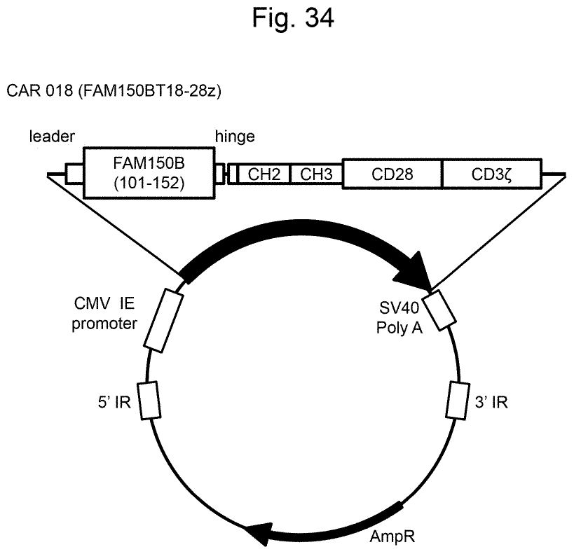

FIG. 34 shows a vector map of CAR 018 (FAM150BT18-28z), and the DNA sequence of a translation region of CAR 018 is represented by SEQ ID NO: 164.

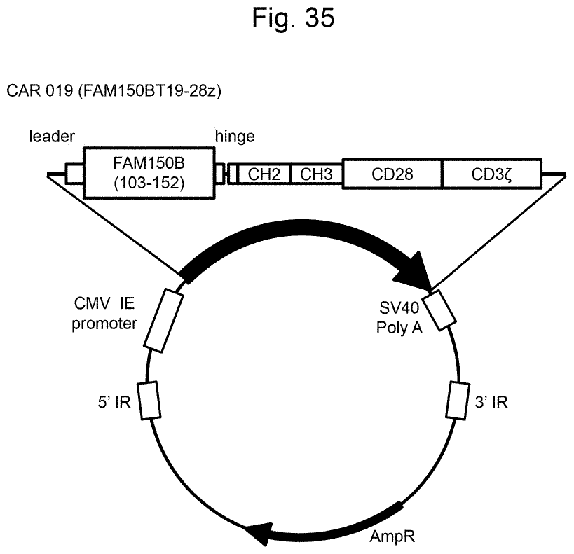

FIG. 35 shows a vector map of CAR 019 (FAM150BT19-28z) and the DNA sequence of a translation region of CAR 019 is represented by SEQ ID NO: 165.

### Example 15

### Comparison of Antitumor Activity of FAM150BT14, FAM150BT15, and FAM150BT17 to FAM150BT19 Truncated CAR-Expressing T Cells

In accordance with the method of CAR-T cell culture and proliferation in Example 2, CAR-T cells were prepared using the CAR 015 to CAR 019-expressing plasmids prepared in Example 14.

CAR-T cells obtained by electrical introduction into PBMCs with CAR 015 to CAR 019 and T cell culture and proliferation are described as follows.
CAR-T 015: FAM150B truncated T14 CD28-type CAR-T (FAM150BT14-CD28z CAR-T)
CAR-T 016: FAM1150B truncated T15 CD28-type CAR-T (FAM150BT15-CD28z CAR-T)
CAR-T 017: FAM150B truncated T17 CD28-type CAR-T (FAM150BT17-CD28z CAR-T)
CAR-T 018: FAM150B truncated TIS CD28-type CAR-T (FAM150BT18-CD28z CAR-T)
CAR-T 019: FAM150B truncated T19 CD28-type CAR-T (FAM150BT19-CD28z CAR-T)

The amino acid sequence of CAR 015 and the nucleotide sequence encoding the same are represented by SEQ ID NO: 166 and SEQ ID NO: 161, respectively.

The amino acid sequence of CAR 016 and the nucleotide sequence encoding the same are represented by SEQ ID NO: 167 and SEQ ID NO: 162, respectively.

The amino acid sequence of CAR 017 and the nucleotide sequence encoding the same are represented by SEQ ID NO: 168 and SEQ ID NO: 163, respectively.

The amino acid sequence of CAR 018 and the nucleotide sequence encoding the same are represented by SEQ ID NO: 169 and SEQ ID NO: 164, respectively.

The amino acid sequence of CAR 019 and the nucleotide sequence encoding the same are represented by SEQ ID NO: 170 and SEQ ID NO: 165, respectively.

Table 6 shows CAR expression rates in the CAR-T cells obtained by introduction of CAR-expressing plasmids into PBMCs derived from a healthy adult donor (Donor-1) and culture.

TABLE 6

| Donor | Plasmid | CAR-T | Expression rate |
|---|---|---|---|
| Donor-1 | CAR 015 | CAR-T 015 | 17.6 |
| | CAR 016 | CAR-T 016 | 23.7 |
| | CAR 017 | CAR-T 017 | 31.8 |
| | CAR 018 | CAR-T 018 | 32.3 |
| | CAR 019 | CAR-T 019 | 27.0 |
| | CAR 008 | CAR-T 008 | 26.7 |
| | CAR 006 | CAR-T 006 | 21.4 |

### Measurement of Antitumor Activity of CAR-T

In order to measure antitumor activity of CAR-T 015 to CAR-T 019 obtained above, co-culture with solid tumor cells was conducted. In this example, passaged cell lines of SH-SY5Y, NB-1, IMR32, and MDA-MB231 ffLuc were used as target tumor cells.

Activity evaluation was performed in accordance with the method of Example 3 using BD Accuri™C6 Plus flow cytometer (BD Biosciences). MDA-MB231 ffLuc cells were analyzed by flow cytometry using 2 μl of PE Anti-Human EGFR antibody (Miltenyi Biotec) instead of PE anti-human Ganglioside GD2 antibody, and the number of EGFR-positive tumor cells was determined based on the number of counting beads.

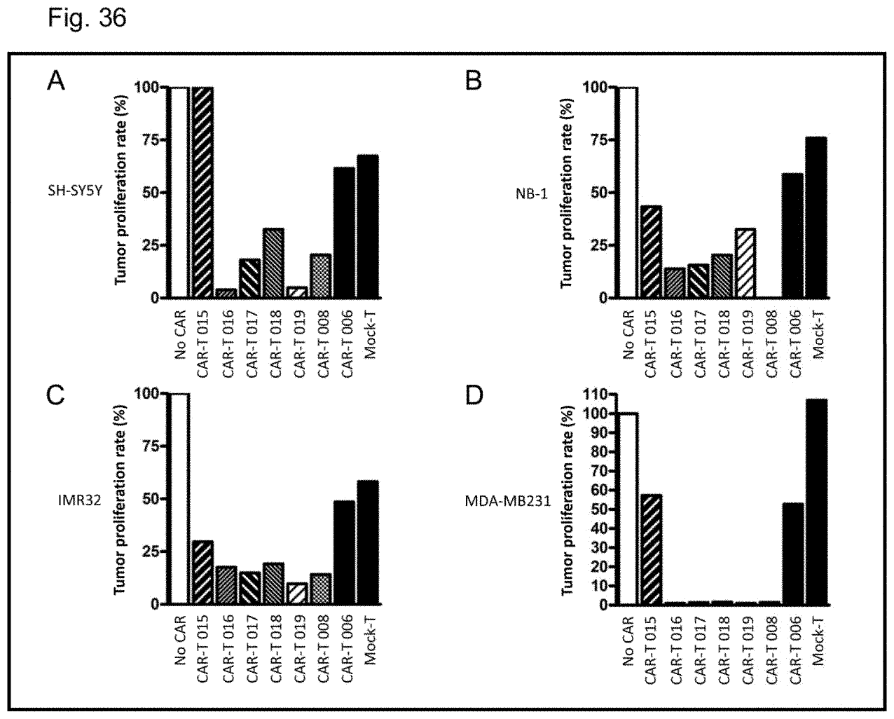

FIG. 36 shows antitumor activity of CAR-T015 to CAR-T 019. CAR-T 008, and CAR-T 006 or mock-T cells derived from Donor-1 against SH-SY5Y, NB-1, IMR32, and MDA-MB231 ffLuc. As a result, the effects of CAR-T 016 to CAR-T 019 to kill SH-SY5Y, NB-1, IMR32, and MDA-MB231 ffLuc were verified. Activity thereof was not significantly different from high-level antitumor activity of CAR-T 008 observed in Example 8.

This example indicates that antitumor activity of CAR-T 016 to CAR-T 019 against neuroblastoma cell lines and breast cancer cell lines is substantially equivalent to that of CAR-T 008.

### Example 16

### Preparation of FAM150B Truncated CH2CH3-Deletion CAR-Expressing Plasmid

With the use of CAR 012 (FAM150BTr-8αBBz) prepared in Example 12 as a template, a CH2CH3-deletion expression plasmid CAR 020 was prepared. A forward primer having the sequence represented by SEQ ID NO: 171 and a reverse primer having the sequence represented by SEQ ID NO: 172 were used for inverse-PCR.

CAR 012 was adjusted to 50 ng/μl and used as a template, and the concentration of each PCR primer was adjusted to 0.2 μM in the reaction solution. Inverse-PCR was performed using KOD-Plus-Mutagenesis Kit (Toyobo Co. Ltd.) with the reaction composition designated in the instructions of the kit and with a cycle consisting of (i) 94° C. for 2 minutes, (ii) 98° C. for 10 seconds, and (iii 68°) C for 7 minutes, and a cycle of steps (ii) and (iii) was repeated 10 times.

After the PCR reaction, an aliquot of the sample was separated via 1% agarose gel electrophoresis, amplification of a linear plasmid of the size of interest was confirmed, the remaining sample after PCR was treated with DpnI in accordance with the instructions of the KOD-Plus-Mutagenesis Kit (Toyobo Co. Ltd.), and methylated template plasmid CAR 012 was cleaved and removed. Thereafter, the linear plasmid was phosphorylated with T4 Polynucleotide Kinase included in the kit and self-ligated to form a cyclic plasmid. Thereafter, E. coli DH5a (Toyobo Co. Ltd.) cells were transformed using the cyclic plasmid and then cultured on an LB agar medium containing 100 μg/ml carbenicillin for about 16 hours.

The appeared colonies were further cultured in an LB liquid medium containing 100 μg/ml carbenicillin for about 16 hours. Plasmids were purified from the cultured E. coli cells using QIAprep Spin Miniprep Kit (Qiagen), nucleotide sequences were determined, and a plasmid in which target nucleotide sequence deletion was observed was obtained as FAM150B truncated CH2CH3-deletion CAR-expressing plasmid (CAR 020; FAM150BTr-BBz dCH2CH3).

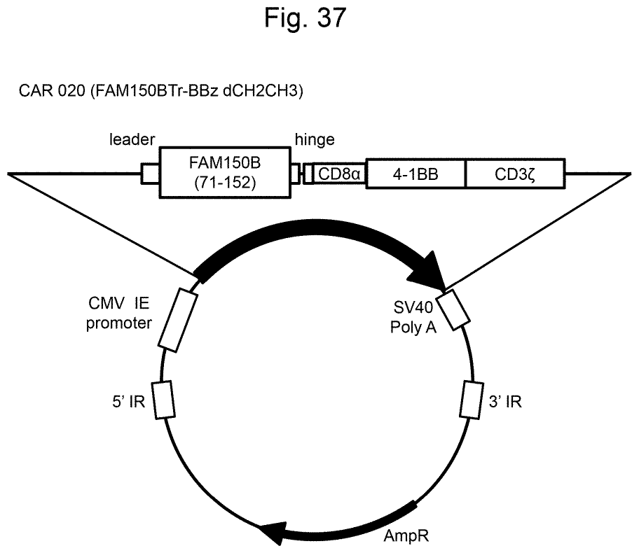

FIG. 37 shows a vector map of CAR 020 (FAM150BTr-BBz dCH2Ch3), and the DNA sequence of a translation region of CAR 020, and the amino acid sequence of CAR 020 are represented by SEQ ID NO: 173 and SEQ ID NO: 174, respectively.

### Culture Method

In accordance with the method of CAR-T cell culture and proliferation in Example 2, CAR-T cells were prepared using the CD19 scFv-type CAR-expressing plasmid (CAR 006; CD19-28z), CAR 012 (FAM150BTr-8αBBz) prepared in Example 12, and CAR 020 expression plasmid described above. The CAR-T cells prepared using CAR 020 were subjected to flow cytometric analysis with the use of 5 μl of Mouse Anti-Human IgG1 Hinge-FITC Secondary Antibody (ABGENT Inc) instead of FITC Goat Anti-Human IgG (H+L) antibody (Jackson ImmunoResearch Inc).

Table 7 shows CAR expression rates in the CAR-T cells obtained by introduction of CAR-expressing plasmids into PBMCs derived from a healthy adult donor (Donor-1) and culture.

TABLE 7

| Donor | Plasmid | CAR-T | Expression rate |
|---|---|---|---|
| Donor-1 | CAR 012 | CAR-T 012 | 17.5 |
| | CAR 020 | CAR-T 020 | 29.3 |
| | CAR 006 | CAR-T 006 | 31.8 |

### Measurement of Antitumor Activity of CAR-T

In order to measure antitumor activity of CAR-T 006, CAR-T 012, and CAR-T 020 obtained above, co-culture with solid tumor cells was conducted. In this example, passaged cell lines of SH-SY5Y, NB-1, IMR32, and MDA-MB231 ffLuc were used as target tumor cells.

Activity evaluation was performed in accordance with the method of Example 3 using BD Accuri™C6 Plus flow cytometer (BD Biosciences). MDA-MB231 ffLuc cells were analyzed by flow cytometry using 2 μl of PE Anti-Human EGFR antibody (Miltenyi Biotec) instead of PE anti-human Ganglioside GD2 antibody, and the number of EGFR-positive tumor cells was determined based on the number of counting beads.

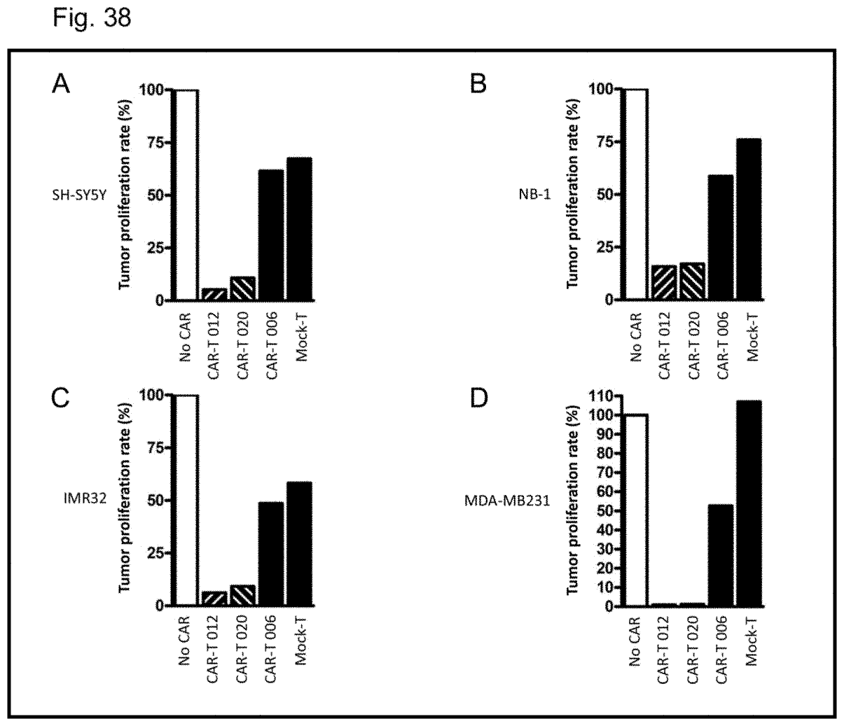

FIG. 38 shows antitumor activity of CAR-T 006, CAR-T 012, and CAR-T 020 or mock-T cells derived from Donor-1 against SH-SY5Y, NB-1, IMR32, and MDA-MB231 ffLuc. As a result, the effects of CAR-T 020 to kill SH-SY5Y, NB-1, IMR32, and MDA-MB231 ffLuc were verified, and activity thereof was equivalent to that of CAR-T 012.

### Example 17

### Preparation of ALK- or LTK-Expressing Plasmid

Preparation of ALK-Expressing Plasmid (pEHX-ALK)

A DNA sequence (HindIII-Kozak-ALK-DraIII; SEQ ID NO: 176) comprising a restriction enzyme HindIII cleavage sequence and a Kozak sequence (SEQ ID NO: 21) added to a region upstream of a sequence from the start codon to a restriction enzyme DraIII cleavage sequence of a translation region of ALK (NCBI Accession Number: NM_004304.5) (928 to 3,198 bp; SEQ ID NO: 175) was designed. Separately, a DNA sequence (DraIII-ALK-TGA-NotI; SEQ ID NO: 178) comprising a stop codon TGA and a restriction enzyme NotI cleavage sequence added to a region downstream of a sequence from the restriction enzyme DraIII cleavage sequence in a translation region of ALK (NCBI Accession Number: NM_004304.5) (3,190 to 5,790 bp; SEQ ID NO: 177) was designed. The two designed DNA sequences were artificially synthesized and incorporated into a pEX-K4J2 vector (synthesis was commissioned to Eurofins Genomics K.K.).

The pEX-K4J2 vector (1 μg equivalent) comprising the sequence of SEQ ID NO: 176 incorporated thereinto was digested with restriction enzymes HindIII, DraIII and SphI at 37° C. for about 3 hours. The pEX-K4J2 vector comprising the sequence of SEQ ID NO: 178 incorporated thereinto was digested with restriction enzymes DraIII, NotI, and SphI at 37° C. for about 3 hours. After the enzyme treatment, the reaction solution was separated via 1% agarose gel electrophoresis, and the enzyme-treated fragment for SEQ ID NO: 176 with the theoretical value of 2,600 bp and the enzyme-treated fragment for SEQ ID NO: 178 with the theoretical value of 2,279 bp were cleaved from the gel and purified using NucleoSpin Gel and PCR Clean-up® (MACHEREY-NAGEL, Takara Bio Inc.).

Separately, the Mammalian PowerExpress System vector pEHX1.2® (Toyobo Co. Ltd.) used as a host vector was digested with restriction enzymes HindIII and NotI at 37° C. for about 2 hours, and a fragment on the vector side was purified in the same manner as described above.

Thereafter, enzyme-treated fragments of the sequences of SEQ ID NO: 176 and SEQ ID NO:178, and pEHX1.2 were ligated using DNA Ligation Kit (Mighty Mix, Takara Bio Inc.). E. coli DH5a (Toyobo Co. Ltd.) cells were transformed using the ligated cyclic plasmid and then cultured on an LB agar medium containing 100 μg/ml carbenicillin for about 16 hours.

The appeared colonies were further cultured in an LB liquid medium containing 100 μg/ml carbenicillin for about 16 hours. Plasmids were purified from the cultured E. coli cells using QIAprep Spin Miniprep Kit (Qiagen), nucleotide sequences were determined, and a plasmid in which target nucleotide sequence insertion had been observed was obtained as an ALK-expressing plasmid (pEHX-ALK). Nucleotide sequence analysis was commissioned to Eurofins Genomics K.K.

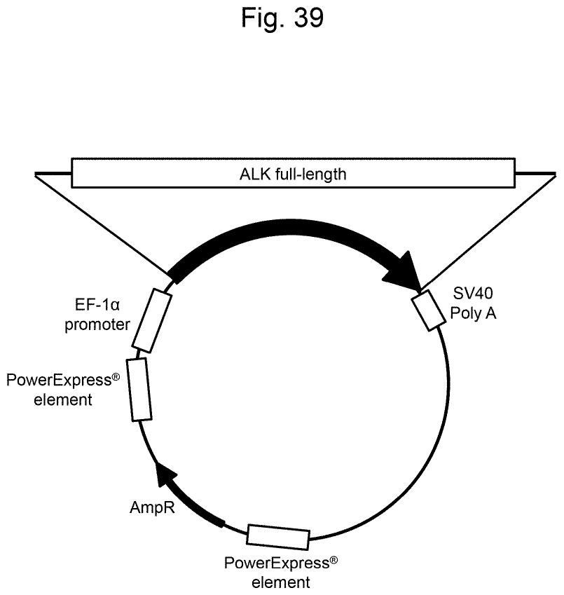

FIG. 39 shows a vector map of pEHX-ALK, and the DNA sequence of a translation region of pEHX-ALK is represented by SEQ ID NO: 179.

Preparation of LTK-Expressing Plasmid (pEHX-LTK)

With the use of LTK 001 prepared in Example 4 as a template and the PCR primers represented by SEQ ID NO: 180 and SEQ ID NO: 181, a restriction enzyme NotI cleavage sequence was added to a region upstream of the Kozak sequence (SEQ ID NO: 21) of LTK 001, and a region up to the restriction enzyme MeI cleavage sequence in the LTK sequence (NotI-Kozak-LTK-NheI; SEQ ID NO: 182) was amplified by PCR. PCR was carried out with the use of PrimeSTAR Max DNA polymerase (Takara Bio Inc.) with a cycle consisting of 98° C. for 10 seconds, 55° C. for 5 seconds, and 72° C. for 10 seconds, which was repeated 35 times.

After the PCR reaction, the sample was separated via 1% agarose gel electrophoresis, and the polynucleotide of SEQ ID NO: 182 was purified using NucleoSpin Gel and PCR Clean-up®(MACHEREY-NAGEL, Takara Bio Inc.). Thereafter, the resultant was incorporated into a pCR-BluntII-TOPO vector via blunt-end cloning using Zero Blunt TOPO PCR Cloning Kit® (Thermo Fisher Scientific). E. coli DH5α (Toyobo Co. Ltd.) cells were transformed using the cyclic plasmid comprising the sequence of SEQ ID NO: 182 incorporated thereinto and then cultured on an LB agar medium containing 50 μg/ml kanamycin for about 16 hours.

The appeared colonies were further cultured in an LB liquid medium containing 50 μg/ml carbenicillin for about 16 hours. Plasmids were purified from the cultured E. coli cells using QIAprep Spin Miniprep Kit (Qiagen)(pCR-BluntII-NotI-Kozak-LTK-NheI).

Separately, a DNA sequence (NheI-LTK-TAA-XbaI; SEQ ID NO: 183) comprising a stop codon TAA and a restriction enzyme XbaI cleavage sequence added to a region downstream of a region from a restriction enzyme NheI cleavage sequence to the stop codon TGA of LTK (NCBI Accession Number: NM_002344.5) was designed and codon-optimized for human. The designed DNA sequence was artificially synthesized and incorporated into a pEX-K4J2 vector (synthesis was commissioned to Eurofins Genomics K.K.).

With the use of the pEX-K4J2 vector comprising the sequence of SEQ ID NO: 183 incorporated therein as a template and PCR primers represented by SEQ ID NO: 184 and SEQ ID NO: 185, a sequence (NheI-LTK-TGATAA-NotI; SEQ ID NO: 186) comprising a restriction enzyme NotI cleavage sequence added to a region downstream of a region from a restriction enzyme NheI cleavage sequence to 2 stop codons (TGATAA) of LTK was amplified by PCR. PCR was carried out with the use of PrimeSTAR Max DNA polymerase (Takara Bio Inc.) with a cycle consisting of 98° C. for 10 seconds, 55° C. for 5 seconds, and 72° C. for 10 seconds, which was repeated 35 times.

After the PCR reaction, the sample was separated via 1% agarose gel electrophoresis, and the polynucleotide of SEQ ID NO: 186 was purified using NucleoSpin Gel and PCR Clean-up® (MACHEREY-NAGEL. Takara Bio Inc.). Thereafter, the resultant was incorporated into a pCR-BluntII-TOPO vector via blunt-end cloning using Zero Blunt TOPO PCR Cloning Kit® (Thermo Fisher Scientific). E. coli DH5a (Toyobo Co. Ltd.) cells were transformed using the cyclic plasmid comprising the sequence of SEQ ID NO: 186 incorporated thereinto and then cultured on an LB agar medium containing 50 μg/ml kanamycin for about 16 hours.

The appeared colonies were further cultured in an LB liquid medium containing 50 μg/ml carbenicillin for about 16 hours. Plasmids were purified from the cultured E. coli cells using QIAprep Spin Miniprep Kit (Qiagen)(pCR-BluntII-NheI-LTK-NotI).

pCR-BluntII-NotI-Kozak-LTK-NheI (1 μg equivalent) was digested with restriction enzymes HindIII and NheI at 37° C. for about 3 hours. Separately, pCR-BluntII-NheI-LTK-NotI (1 μg equivalent) was digested with restriction enzymes NheI and NotI at 37° C. for 2 hours. After the enzyme treatment, the reaction solution was separated via 1% agarose gel electrophoresis, and a cleavage fragment of SEQ ID NO: 182 or SEQ ID NO: 186 was cleaved from the gel and purified using NucleoSpin Gel and PCR Clean-up® (MACHEREY-NAGEL, Takara Bio Inc.).

Separately, the Mammalian PowerExpress System vector pEHX1.2® (Toyobo Co. Ltd.) used as a host vector was digested with restriction enzymes HindIII and NotI at 37° C. for about 2 hours, and a fragment on the vector side was purified in the same manner as described above.

Thereafter, enzyme-treated fragments of the sequences of SEQ ID NO: 182 and SEQ ID NO: 186, and pEHX1.2 were ligated using DNA Ligation Kit (Mighty Mix, Takara Bio Inc.). E. coli DH5a (Toyobo Co. Ltd.) cells were transformed using the ligated cyclic plasmid and then cultured on an LB agar medium containing 100 μg/ml carbenicillin for about 16 hours.

The appeared colonies were further cultured in an LB liquid medium containing 100 μg/ml carbenicillin for about 16 hours. Plasmids were purified from the cultured E. coli cells using QIAprep Spin Miniprep Kit (Qiagen), nucleotide sequences were determined, and a plasmid in which target nucleotide sequence insertion was observed was obtained as an LTK-expressing plasmid (pEHX-LTK). Nucleotide sequence analysis was commissioned to Eurofins Genomics K.K.

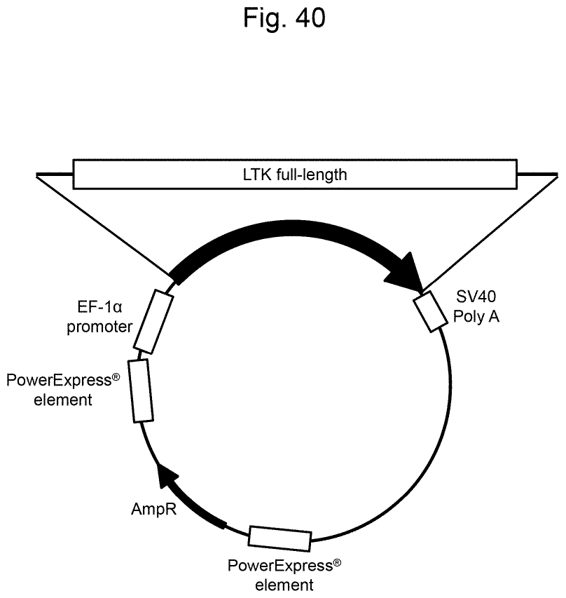

FIG. 40 shows a vector map of pEHX-LTK, and the DNA sequence of a translation region of pEHX-LTK is represented by SEQ ID NO: 187.

### Example 18

### Isolation of ALK- or LTK-Expressing Stable CHO-K1 Cells

Transfection into CHO-K1 Cells

The 2 types of plasmids prepared in Example 17 (pEHX-ALK and pEHX-LTK) were introduced into CHO-K1 cells using Lipofectamine 3000 Transfection Reagent® (Thermo Fisher Scientific). Specifically, CHO-K1 cells were seeded into 4 wells of a 12-well treated culture plate at 2×10⁵ cells·2 ml/well on the day before gene transfection (Day −1). Ham's F-12 medium with L-glutamine and phenol red (FUJIFILM Wako Pure Chemical Corporation) containing penicillin-streptomycin (FUJIFILM Wako Pure Chemical Corporation) and 10% FBS (GE Healthcare Japan) was used. On the following day (Day 0), a mixture of the plasmid solution (1 μg equivalent), 2 μl of P3000 Reagent (Thermo Fisher Scientific), and 50 μl of Opti-MEM I Reduced-Serum Medium (Thermo Fisher Scientific) was added to a mixture of 3 μl of Lipofectamine 3000 Reagent and 50 μl of the Opti-MEM I Reduced-Serum Medium, and the mixture was subjected to the reaction at room temperature for 15 minutes. Thereafter, the reaction solution was added to each well of the 12-well treated culture plate for gene transfection.

### Drug Selection and Cloning of Transfected CHO-K1 Cells

The cells were collected using 0.25 w/v % trypsin-1 mmol/1 EDTA·4Na solution (FUJIFILM Wako Pure Chemical Corporation) 24 hours after gene transfection (Day 1), and the CHO-K1 cells collected from 2 wells were transferred to a 100-mm dish. In addition, puromycin dihydrochloride (Thermo Fisher Scientific) was added at 10 μg/ml, the medium was exchanged with a fresh medium every 3 or 4 days, and culture was continued up to Day 12. On Day 12, the resultant was seeded on a 96-well treated plate at 1 cell/well. The cells were transferred to a 24-well treated plate on Day 29 for expansion culture.

Screening of cloned cells based on mRNA expression level

Some cells were collected on Day 33, and RNA was extracted using RNeasy Mini Kit (Qiagen). The extracted RNA was subjected to reverse transcription using PrimeScript RT Master Mix (Perfect Real Time, Takara Bio Inc.) to prepare cDNA. Using the resulting cDNA as a template, quantitative PCR was performed by 7500 Fast real-time PCR System (Thermo Fisher Scientific) with the use of TB Green Premix Ex Taq II (Takara Bio Inc.) in combination with primers for ALK amplification (SEQ ID NO: 188 and SEQ ID NO: 189) or primers for LTK amplification (SEQ ID NO: 190 and SEQ ID NO: 191). The mRNA expression level in quantitative PCR was determined by correcting the number of cycles (Ct value) using 18S ribosomal RNA as the internal reference gene, and clones having many copies of mRNA were selected based on the results of relative quantification of ALK and LTK.

### Confirmation of ALK and LTK Expression on Selected Clone Cell Surfaces

There is no adequate fluorescence-labeled antibody that specifically detects the extracellular domain of ALK or LTK. Thus, flow cytometric analysis was performed using binding affinity of ALK or LTK to its ligand FAM150B. Specifically, a suspension of 0.6 to 1.8×10⁶ cells was centrifuged to remove the supernatant, the FAM150B-His protein prepared in Example 5 (about 0.2 to 0.3 μmol equivalent) was added, and the resultant was incubated at 37° C. for 30 minutes. Thereafter, the product was washed with 1 ml of D-PBS containing 1% FBS, centrifuged to remove the supernatant, and then subjected to the same washing procedure 3 times. Thereafter, 5 μl of PE (phycoerythrin) anti-His tag antibody (BioLegend) was added, and the resultant was incubated at 4° C. under shading conditions for 20 minutes. Thereafter, the resultant was washed with 1 ml of D-PBS containing 1% FBS and centrifuged to remove the supernatant. The sample re-suspended in 500 μl of D-PBS containing 1% FBS was assayed using BD Accuri C6 Plus (BD Biosciences), and the obtained data were analyzed using FlowJo (BD Biosciences).

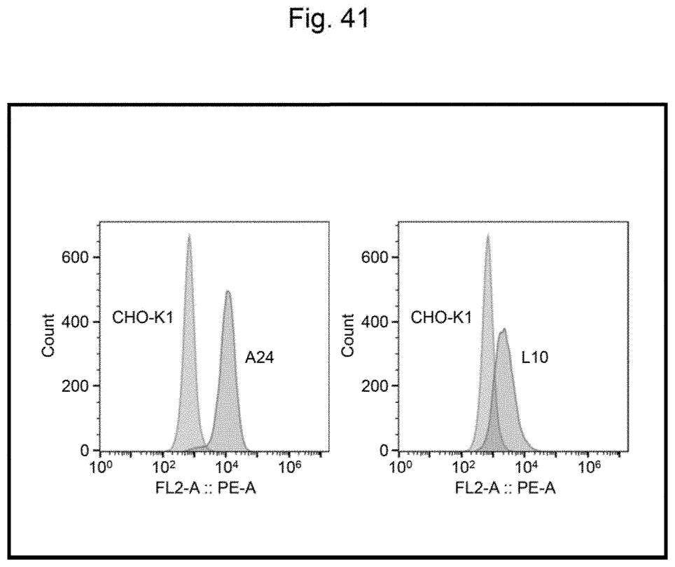

FIG. 41 shows the results of flow cytometric analysis using binding affinity of clones to the FAM150B protein. PE fluorescence intensity exhibited by the clone comprising ALK gene introduced thereinto (A24) and the clone comprising LTK gene introduced thereinto (L10) is shifted significantly to the right, compared with the histogram of the control group (CHO-K1). It was thus demonstrated that A24 expresses ALK and L10 expresses LTK constitutionally on their cell surfaces.

### Example 19

### Preparation of FAM150B Truncated CH2CH3-Deletion CD28-Type CAR-Expressing Plasmid

CAR 008 prepared in Example 7 was used as a template, a FAM150B truncated CH2CH3-deletion CD28-type CAR-expressing plasmid (CAR 021) was prepared by inverse-PCR. The forward primer (SEQ ID NO: 171) and the reverse primer (SEQ ID NO: 172) were used for inverse-PCR. CAR 008 was adjusted to 50 ng/μl and used as a template, and the concentration of each PCR primer was adjusted to 0.2 μM in the reaction solution. Inverse-PCR was performed using KOD-Plus-Mutagenesis Kit (Toyobo Co. Ltd.) with the reaction composition designated in the instructions of the kit and with a cycle consisting of (i) 94° C. for 2 minutes, (ii) 98° C. for 10 seconds, and (iii) 68° C. for 7 minutes, and a cycle of steps (ii) and (iii) was repeated 10 times.

After the PCR reaction, an aliquot of the sample was separated via 1% agarose gel electrophoresis, amplification of the linear plasmid of the size of interest was confirmed, the remaining sample after PCR was treated with DpnI in accordance with the instructions of the kit, and methylated template plasmid CAR 008 was cleaved and removed. Thereafter, the linear plasmid was phosphorylated with T4 Polynucleotide Kinase included in the kit and self-ligated to form a cyclic plasmid. Thereafter, E. coli DH5a (Toyobo Co. Ltd.) cells were transformed using the cyclic plasmid and then cultured on an LB agar medium containing 100 μg/ml carbenicillin for about 16 hours.

The appeared colonies were further cultured in an LB liquid medium containing 100 μg/ml carbenicillin for about 16 hours. Plasmids were purified from the cultured E. coli cells using QIAprep Spin Miniprep Kit (Qiagen), nucleotide sequences were determined, and a plasmid in which target nucleotide sequence deletion was observed was obtained as a FAM150B truncated CH2CH3-deletion CD28-type CAR-expressing plasmid (CAR 021; FAM150BTr-28z dCH2CH3).

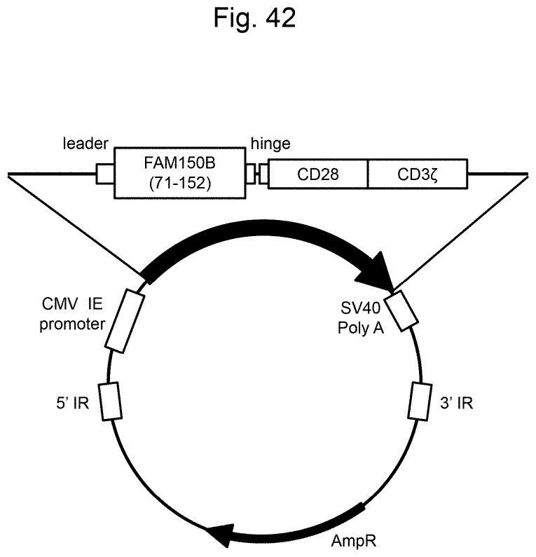

FIG. 42 shows a vector map of CAR 021 (FAM150BTr-28z dCH2CH3), and the DNA sequence of a translation region of CAR 021 is represented by SEQ ID NO: 192.

### Example 20

Preparation of Mouse ALK48 scFv-Type CD28-Type CH2CH3-Deletion CAR-Expressing Plasmid (CAR 022; ALK48-28z dCH2CH3)

CAR021 prepared in Example 19(about 1 μg equivalent) was digested with restriction enzymes XhoI and DraIII (New England Biolabs) at 37° C. for about 2 hours. Also, the pEX-K4J1 vector (about 1 μg equivalent) comprising the sequence of SEQ ID NO: 8, which was designed and artificially synthesized in Example 1, incorporated thereinto was digested with restriction enzymes XhoI and DraIII at 37° C. for about 2 hours.

After the enzyme treatment, the reaction solution was separated via 1% or 2% agarose gel electrophoresis, the enzyme-treated CAR 021 fragment (on the vector side) and the artificially synthesized gene-inserted fragment (SEQ ID NO: 8) cleaved from the pEX-K4J1 vector were removed from the gel, and the fragments were purified using NucleoSpin Gel and PCR Clean-up® (MACHEREY-NAGEL, Takara Bio Inc.). The purified vector fragment was ligated to the purified insert fragment using the DNA ligation kit (Mighty Mix, Takara Bio Inc.). Thereafter, E. coli DH5α (Toyobo Co. Ltd.) cells were transformed using the ligated cyclic plasmid and then cultured on an LB agar medium containing 100 μg/ml carbenicillin for about 16 hours.

The appeared colonies were further cultured in an LB liquid medium containing 100 μg/ml carbenicillin for about 16 hours. Plasmids were purified from the cultured E. coli cells using QIAprep Spin Miniprep Kit (Qiagen), nucleotide sequences were determined, and a plasmid in which target nucleotide sequence insertion was observed was obtained as a mouse ALK48 scFv-type CD28-type CH2CH3-deletion CAR-expressing plasmid (CAR 022: ALK48-28z dCH2CH3). Nucleotide sequence analysis was commissioned to Eurofins Genomics K.K.

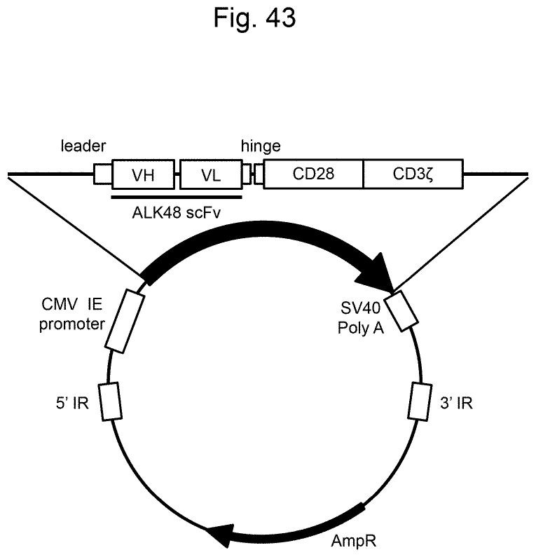

FIG. 43 shows a vector map of CAR 022 (ALK48 scFv-28z dCH2CH3), and the DNA sequence of a translation region of CAR 022 is represented by SEQ ID NO: 193.

### Example 21

### Culture and Proliferation of CAR-T Cells

In accordance with the method of CAR-T cell culture and proliferation in Example 2, CAR-T cells were prepared using the CAR 021- or CAR 022-expressing plasmid prepared in Examples 19 and 20.

### Day 16: Evaluation of CAR Expression Rate

The number of CAR-T cells into which the CAR 021- or CAR 022-expressing plasmid had been introduced was counted, and 1 to 2×10⁵ cells were subjected to flow cytometric analysis to evaluate the CAR expression rates in the ALK CAR-T cells.

1 to 2×10⁵ cells were collected and washed with 1 ml of D-PBS containing 1% FBS, 100 μl of ALK 001P prepared in Example 5 was added, and the resultant was incubated at 37° C. for 30 minutes. Thereafter, the cells were washed with 1 ml of D-PBS containing 1% FBS, centrifuged to remove the supernatant, and then subjected to the same washing procedure 3 times. Thereafter, 5 μl of PE anti-His tag antibody (BioLegend) and 5 μl of APC Anti-Human CD3 antibody (Miltenyi Biotec) were added to prepare a suspension, and the antibody labeling reaction was conducted at 4° C. under shading conditions for 20 minutes. Thereafter, the cells were washed with 1 ml of D-PBS containing 1% FBS, and precipitated by centrifugation to remove the supernatant. Thereafter, the sample re-suspended in 500 μl of D-PBS containing 1% FBS was assayed using BD Accuri C6 Plus (BD Biosciences) to determine CAR 021 or CAR 022 positive rate.

The CAR-T cells obtained by gene introduction through electrical pulses into PBMCs using CAR 021 or CAR 022 and T cell culture and proliferation are described herein as follows. CAR-T 021: FAM150B CD28-type CH2CH3-deletion CAR-T (FAM150B-28z dCH2CH3 CAR-T)

CAR-T 022: Mouse CD28-Type CH2CH3-Deletion CAR-T (ALK scFv-28z dCH2CH3 CAR-T)

The amino acid sequence of CAR 021 and the nucleotide sequence encoding the same are represented by SEQ ID NO: 194 and SEQ ID NO: 192, respectively.

The amino acid sequence of CAR 022 and the nucleotide sequence encoding the same are represented by SEQ ID NO: 195 and SEQ ID NO: 193, respectively.

Table 8 shows CAR expression rates (%) in CAR-T cells obtained from PBMCs derived from a healthy adult donor.

TABLE 8

| Donor | Plasmid | CAR-T | Expression rate |
|---|---|---|---|
| Donor-1 | CAR 021 | CAR-T 021 | 24.1 |
| | CAR 022 | CAR-T 022 | 24.4 |

### Example 22

### Evaluation of Cytotoxic Activity of CAR-T 021 or CAR-T 022

The clones A24 and L10 selected in Example 18 and CHO-K1 cells were seeded as target (1) cells at 5,000 cells/well on E-Plate VIEW (ACEA Biosciences). The plate was mounted on xCELLigence DP (ACEA Biosciences), and about 24 hours later, CAR-T 021, CAR-T 022 and mock-T cells (T cells into which no genes had been introduced) were added as effector (E) cells at the E:T ratios of 40:1 (200,000 cells:5,000 cells), 20:1 (100,000 cells:5,000 cells), and 10:1 (50,000 cells:5,000 cells). About 48 hours after the addition of effector cells, the cell index: i.e., transition in the number of adhesive cells, was evaluated. Evaluation was performed using Ham's F-12 medium with L-glutamine and phenol red (FUJIFILM Wako Pure Chemical Corporation) containing penicillin-streptomycin (FUJIFILM Wako Pure Chemical Corporation) and 10% FBS (GE Healthcare Japan).

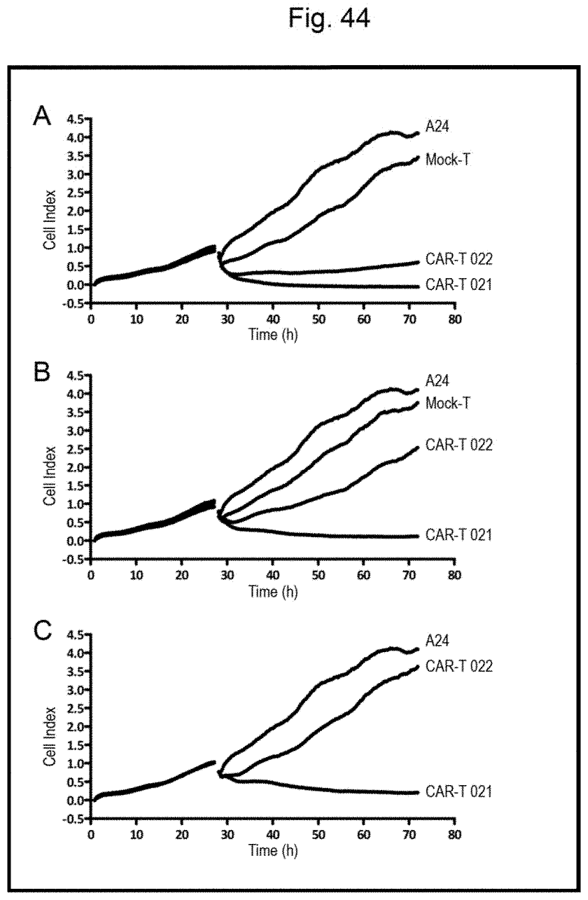

FIG. 44 shows transitions in the cell index over time when CAR-T 021, CAR-T 022, or mock-T cells are added to A24 cells expressing high levels of ALK. While CAR-T 021 and CAR-T 022 significantly lowered the cell index of A24 cells in an E:T ratio-dependent manner, mock-T cells did not affect the cell index of A24. The results demonstrate that CAR-T 021 and CAR-T 022 have specific cytotoxic activity against cells expressing high levels of ALK and that cytotoxic activity of CAR-T 021 is higher.

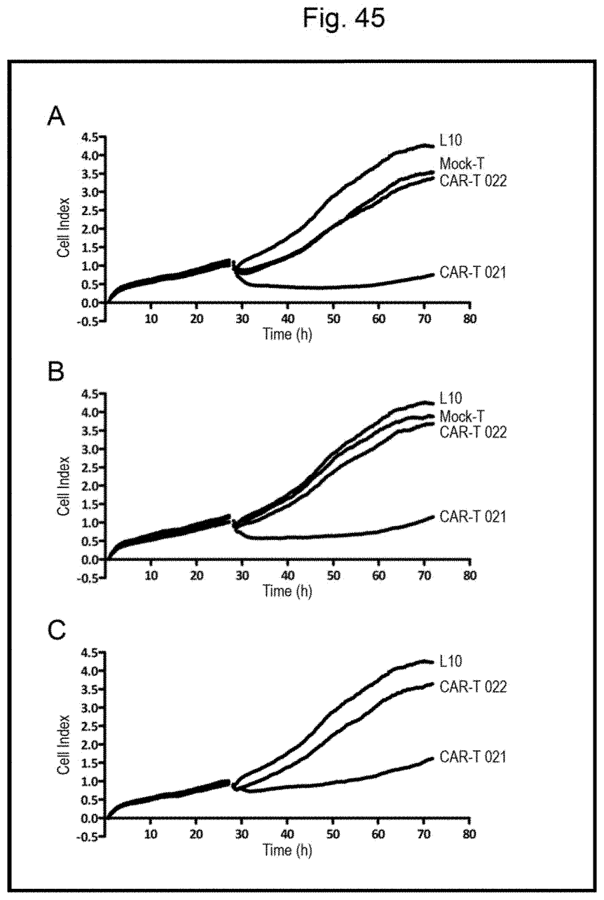

FIG. 45 shows transitions in the cell index over time when CAR-T 021, CAR-T 022, or mock-T cells are added to L10 cells expressing high levels of LTK. While CAR-T 021 significantly lowered the cell index of L10 cells in an E:T ratio-dependent manner, CAR-T 022 and mock-T cells did not affect the cell index of L10. The results demonstrate that CAR-T 021 has specific cytotoxic activity against cells expressing high levels of LTK.

In this example, FAM150B CAR-T was found to specifically recognize both the cells expressing high levels of ALK and the cells expressing high levels of LTK and exert cytotoxic activity thereon because of the ligand-type properties. In contrast, ALK scFv-type CAR-T was found to selectively act on the cells expressing high levels of ALK.

In this example, in addition, antitumor activity of FAM150B truncated CH2CH3-deletion CAR-T against neuroblastoma cell line and breast cancer cell line was found to be substantially equivalent to that of an FAM150B truncated form having CH2CH3.

All publications, patents, and patent applications cited herein are incorporated herein by reference in their entirety.

1. A polynucleotide encoding a chimeric antigen receptor (CAR) protein comprising a target binding domain that binds to an extracellular ligand binding region of anaplastic lymphoma kinase (ALK), a transmembrane domain, and an intracellular signaling domain, wherein the target binding domain is selected from among FAM150A, FAM150B, and fragments thereof binding to the extracellular ligand binding region of ALK.

2. The polynucleotide according to claim 1, wherein the target binding domain is a truncated fragment of FAM150A and/or FAM150B.

3. The polynucleotide according to claim 1, wherein the target binding domain is a polypeptide consisting of an amino acid sequence selected from the group consisting of SEQ ID NO: 154 (FAM150A), SEQ ID NO: 146 (TrFAM150A), SEQ ID NO: 155 (FAM150B), and SEQ ID NO: 148 (TrFAM150B).

4. The polynucleotide according to claim 1, wherein the target binding domain is a polypeptide consisting of an amino acid sequence having 90% or higher sequence identity to the amino acid sequence represented by SEQ ID NO: 146 (TrFAM150A) or SEQ ID NO: 148 (TrFAM150B).

5. A vector comprising the polynucleotide according to claim 1.

6. A genetically modified cell comprising the polynucleotide according to claim 1.

7. The cell according to claim 6, which expresses a CAR protein binding to an ALK-expressing cell on a cell membrane.

8. A method for preparing a CAR protein-expressing cell comprising introducing the polynucleotide according to claim 1.

9. The method according to claim 8, wherein the polynucleotide or the vector is introduced into the cell by the transposon method.

10. The method according to claim 9, wherein the transposon method is the piggyBac method.

11. A kit comprising the vector according to claim 5 used for preparing a CAR protein-expressing cell targeting an ALK-expressing cell.

12. A therapeutic agent for a disease associated with an ALK-expressing cell comprising the cell according to claim 6.

13. A pharmaceutical composition comprising the therapeutic agent according to claim 12 and a pharmaceutically acceptable carrier.

14. The therapeutic agent according to claim 12, wherein the disease associated with an ALK-expressing cell is a solid tumor selected from among neuroblastoma, breast cancer, uterine cancer, endometrial cancer, ovarian cancer, melanoma, astroglioma, Ewing's sarcoma, glioblastoma, retinoblastoma, rhabdomyoblastoma, non-small cell lung cancer, prostate cancer, and urothelial cancer.


SEQUENCE LISTING (195 sequences)

SEQ ID NO: 1 - DNA, 387 bp, Homo sapiens
atgcggccccttaagcccggcgcccctttgcccgcactcttcctgctggcgctggctttgtccccgcacggagcccacgggaggccccgggggcgcaggggagcgcgcgtcacggataaggagcccaagccgttgcttttcctccccgcggccggggccggccggactcccagcggctcccggagcgcagaaatattcccaagagactctaacttaaaagacaaattcataaagcatttcacagggccggtcacattttcaccagaatgcagcaaacatttccaccgactctattacaataccagggagtgctcaacgccagcttattacaaaagatgtgctagattgttaacaagattagcagtgagtccactgtgctcccagacc

SEQ ID NO: 2 - DNA, 515 bp, Artificial
  XhoI-leader-FAM150A-Hinge-DraIII
ctcgagctcaagcttcgaattcgccaccatggagttcggactgtcctggctgtttctggtggccatactgaaaggggttcagtgcatgaggccactgaaaccgggagcacctcttcctgccctgtttctgctcgccttggcactgtctcctcatggcgctcatggcagaccacgcgggagaaggggtgctagagtcacggacaaagaacccaaacccctgctgtttcttccagctgcaggggctggacgaactccctcaggcagtcggtcagccgagattttccctcgggatagcaatctcaaggacaagttcatcaagcacttcaccggtcctgtgaccttctctcccgagtgtagcaagcactttcatcgcctgtattacaacacacgtgaatgctccacaccagcctactataagcggtgtgcgaggctcctcactcgcttggccgtaagtccgctttgcagccaaaccgaacccaagtcctgcgataaaacccacacatgtccaccgtgtg

SEQ ID NO: 3 - DNA, 456 bp, Homo sapiens
atgcgcggacccgggcaccccctcctcctggggctgctgctggtgctgggggcggcggggcgcggccgggggggcgcggagccccgggagccggcggacggacaggcgctgctgcggctggtggtggaactcgtccaggagctgcggaagcaccactcggcggagcacaagggcctgcagctcctcgggcgggactgcgccctgggccgcgcggaggcggcggggctggggccttcgccggagcagcgagtggaaattgttcctcgagatctgaggatgaaggacaagtttctaaaacaccttacaggccctctttattttagtccaaagtgcagcaaacacttccatagactttatcacaacaccagagactgcaccattcctgcatactataaaagatgcgccaggcttcttacccggctggctgtcagtccagtgtgcatggaggataagcag

SEQ ID NO: 4 - DNA, 584 bp, Artificial
  XhoI-leader-FAM150B-Hinge-DraIII
ctcgagctcaagcttcgaattcgccaccatggagtttgggctttcctggttgttcctcgtagccatcctcaaaggggttcagtgcatgagagggccaggacatcctctgcttctgggcctgctgctggtgttgggcgctgctggcagaggtcgtggcggagcagagcctagagagccagccgatgggcaagcccttctgcggcttgtggtcgaactcgtgcaggaactgcggaaacaccattccgctgaacacaaaggcctgcaactgctcggaagagactgtgccttgggaagggcagaagcggctggtctgggtccctcaccagaacagcgagtcgagattgtgcctcgcgatctccgcatgaaggacaagttcctgaagcatctgacaggcccgctgtacttcagtcccaagtgtagcaaacactttcatcgcctgtatcacaacacacgggattgcaccatacccgcctattacaagaggtgtgcaaggctgctgactcgattggccgtttctcctgtgtgcatggaggacaaacaggagcccaagagctgcgacaagacgcacacctgtccaccgtgtg

SEQ ID NO: 5 - protein, 271 aa, Artificial
  Humanized ALK48 scFv
MVLLVTSLLLCELPHPAFLLIPDTQVQLQQSGAEVKKPGSSVKVSCKASGYAFSSYISWVRQAPGQGLEWMGGQIYPGDGDTNYAQKFQGRVTITADESTSTAYMELSSLRSEDTAVYYCVRYYYGSSGYFDYWWGQGTMVTVSSGGGGSGGGGSGGGGSEIVLTQSPATLSLSPGERATLSCRASESVDNYGISFAWYQQKPGQAPRLLIYRASRATGIPARFSGSGSGTDFTLTISSLEPEDFAVYYCQQNNKDPPTFFGQGTKLEIKR

SEQ ID NO: 6 - protein, 270 aa, Artificial
  Murine ALK48 scFv
MVLLVTSLLLCELPHPAFLLIPDTQVQLQQSGAELVKPGASVKISCKASGYAFSSYWMNWVKQRPGKGLEWIGQIYPGDGDTTYNGKFKGKATLTADKSSSTVYMQLNSLTSEDSAVYFCVRYYYGSSGYFDYWGQGTTLTVSSGGGGSGGGGSGGGGSDVQMIQTPDSLAVSLGQRATISCRASESVDNYGISFMHWYQQKPGQSPKLLIYRASNLESGIPARFSGSGSRTDFTLTINPVETDDVATYYCQQNNKDPPTFGGGTKLEIK

SEQ ID NO: 7 - DNA, 884 bp, Artificial
  XhoI-leader-humanized ALK48 scFv-Hinge-DraIII
ctcgagctcaagcttcgaattcgccaccatggtgctgctggttacctccctccttctctgcgagttgccgcatcccgcttttctcctgattcccgacacacaggtccagctccagcaatctggggcagaagtgaagaaacccgggtcaagtgtgaaagtctcctgtaaagcaagtggctatgcctttagcagctacatttcctgggttcgacaggcacctggccaaggattggaatggatgggtggacagatctatcctggggatggagacaccaattacgcccagaagtttcagggtcgcgtcacaataacggcagatgagtccactagcactgcctacatggagctgagctctcttcgctcagaggatacagccgtgtactattgcgtacggtactactacggctcttccggctatttcgactattggtggggacagggaaccatggtaaccgtgagttccggcggtggaggaagcggaggcggcgggtcaggaggcggtgggtctgagatcgttctgactcaatctccagctaccctgtctctgagcccaggcgaacgtgctaccttgtcatgcagagctagcgagtctgtggataactacggtatatcctttgcgtggtatcagcagaaacctgggcaagccccaaggctgctgatctatcgggccagtagggctactgggattccggccagatttagcgggagcggcagtggtacagacttcacgctgaccatctcaagccttgaacccgaagatttcgcggtgtattactgccagcagaacaataaggacccacctaccttcttcggccaaggcactaaactggagatcaagagagagcccaagtcctgtgacaagacacacacatgtccaccgtgtg

SEQ ID NO: 8 - DNA, 881 bp, Artificial
  XhoI-leader-murine ALK48 scFv-Hinge-DraIII
ctcgagctcaagcttcgaattcgccaccatggtcctgctcgtcacgtcactgcttctgtgcgagttgcctcatcctgccttcctgctgattcccgatacacaggtgcaacttcagcagtcaggcgcagaactggtcaaaccaggtgctagcgtgaagatctcctgcaaggctagtgggtacgcgttttcctcctactggatgaattgggtgaagcaacgaccaggcaaggggttggagtggataggtcagatttatccaggcgatggggatacaacatacaatgggaagttcaagggaaaggccacacttactgccgacaaatcctctagcaccgtgtacatgcagctgaactccctgacgtccgaagatagcgccgtttacttctgcgtgagatactactacggcagctcagggtatttcgactattggggtcaaggaaccactttgaccgtatcttctggcggaggcggcagtggtggaggtggctcaggaggcggagggtcagacgttcagatgatacagacacctgactctctggccgtttccctcgggcagagagccaccatcagttgtcgcgcttctgagtctgtggataactatgggatcagcttcatgcactggtatcagcagaaacctggccaaagtcccaaactcctcatctatcgggctagcaatctggaaagcggcattccggcacgttttagtggcagcggaagcaggaccgactttaccctgaccattaatccggtggaaactgacgatgtagcaacctactattgccagcagaacaacaaggatccacccacttttggaggtggaacaaagctggagatcaaagagcccaaaagctgtgacaaaacgcacacttgtccaccgtgtg

SEQ ID NO: 9 - DNA, 1746 bp, Artificial
  Car 002 translation region
atggagttcggactgtcctggctgtttctggtggccatactgaaaggggttcagtgcatgaggccactgaaaccgggagcacctcttcctgccctgtttctgctcgccttggcactgtctcctcatggcgctcatggcagaccacgcgggagaaggggtgctagagtcacggacaaagaacccaaacccctgctgtttcttccagctgcaggggctggacgaactccctcaggcagtcggtcagccgagattttccctcgggatagcaatctcaaggacaagttcatcaagcacttcaccggtcctgtgaccttctctcccgagtgtagcaagcactttcatcgcctgtattacaacacacgtgaatgctccacaccagcctactataagcggtgtgcgaggctcctcactcgcttggccgtaagtccgctttgcagccaaaccgaacccaagtcctgcgataaaacccacacatgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 10 - DNA, 1815 bp, Artificial
  Car 003 translation region
atggagtttgggctttcctggttgttcctcgtagccatcctcaaaggggttcagtgcatgagagggccaggacatcctctgcttctgggcctgctgctggtgttgggcgctgctggcagaggtcgtggcggagcagagcctagagagccagccgatgggcaagcccttctgcggcttgtggtcgaactcgtgcaggaactgcggaaacaccattccgctgaacacaaaggcctgcaactgctcggaagagactgtgccttgggaagggcagaagcggctggtctgggtccctcaccagaacagcgagtcgagattgtgcctcgcgatctccgcatgaaggacaagttcctgaagcatctgacaggcccgctgtacttcagtcccaagtgtagcaaacactttcatcgcctgtatcacaacacacgggattgcaccatacccgcctattacaagaggtgtgcaaggctgctgactcgattggccgtttctcctgtgtgcatggaggacaaacaggagcccaagagctgcgacaagacgcacacctgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 11 - DNA, 2115 bp, Artificial
  Car 004 translation region
atggtgctgctggttacctccctccttctctgcgagttgccgcatcccgcttttctcctgattcccgacacacaggtccagctccagcaatctggggcagaagtgaagaaacccgggtcaagtgtgaaagtctcctgtaaagcaagtggctatgcctttagcagctacatttcctgggttcgacaggcacctggccaaggattggaatggatgggtggacagatctatcctggggatggagacaccaattacgcccagaagtttcagggtcgcgtcacaataacggcagatgagtccactagcactgcctacatggagctgagctctcttcgctcagaggatacagccgtgtactattgcgtacggtactactacggctcttccggctatttcgactattggtggggacagggaaccatggtaaccgtgagttccggcggtggaggaagcggaggcggcgggtcaggaggcggtgggtctgagatcgttctgactcaatctccagctaccctgtctctgagcccaggcgaacgtgctaccttgtcatgcagagctagcgagtctgtggataactacggtatatcctttgcgtggtatcagcagaaacctgggcaagccccaaggctgctgatctatcgggccagtagggctactgggattccggccagatttagcgggagcggcagtggtacagacttcacgctgaccatctcaagccttgaacccgaagatttcgcggtgtattactgccagcagaacaataaggacccacctaccttcttcggccaaggcactaaactggagatcaagagagagcccaagtcctgtgacaagacacacacatgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 12 - DNA, 2112 bp, Artificial
  Car 005 translation region
atggtcctgctcgtcacgtcactgcttctgtgcgagttgcctcatcctgccttcctgctgattcccgatacacaggtgcaacttcagcagtcaggcgcagaactggtcaaaccaggtgctagcgtgaagatctcctgcaaggctagtgggtacgcgttttcctcctactggatgaattgggtgaagcaacgaccaggcaaggggttggagtggataggtcagatttatccaggcgatggggatacaacatacaatgggaagttcaagggaaaggccacacttactgccgacaaatcctctagcaccgtgtacatgcagctgaactccctgacgtccgaagatagcgccgtttacttctgcgtgagatactactacggcagctcagggtatttcgactattggggtcaaggaaccactttgaccgtatcttctggcggaggcggcagtggtggaggtggctcaggaggcggagggtcagacgttcagatgatacagacacctgactctctggccgtttccctcgggcagagagccaccatcagttgtcgcgcttctgagtctgtggataactatgggatcagcttcatgcactggtatcagcagaaacctggccaaagtcccaaactcctcatctatcgggctagcaatctggaaagcggcattccggcacgttttagtggcagcggaagcaggaccgactttaccctgaccattaatccggtggaaactgacgatgtagcaacctactattgccagcagaacaacaaggatccacccacttttggaggtggaacaaagctggagatcaaagagcccaaaagctgtgacaaaacgcacacttgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 13 - protein, 581 aa, Artificial
  Car 002 translation region
MEFGLSWLFLVAILKGVQCMRPLKPGAPLPALFLLALALSPHGAHGRPRGRRGARVTDKEPKPLLFLPAAGAGRTPSGSRSAEIFPRDSNLKDKFIKHFTGPVTFSPECSKHFHRLYYNTRECSTPAYYKRCARLLTRLAVSPLCSQTEPKSCDKTHTCPPCDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 604 aa, Artificial
  Car 003 translation region
MEFGLSWLFLVAILKGVQCMRGPGHPLLLGLLLVLGAAGRGRGGAEPREPADGQALLRLVVELVQELRKHHSAEHKGLQLLGRDCALGRAEAAGLGPSPEQRVEIVPRDLRMKDKFLKHLTGPLYFSPKCSKHFHRLYHNTRDCTIPAYYKRCARLLTRLAVSPVCMEDKQEPKSCDKTHTCPPCDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 746 aa, Artificial
  Car 004 translation region
MVLLVTSLLLCELPHPAFLLIPDTQVQLQQSGAEVKKPGSSVKVSCKASGYAFSSYISWVRQAPGQGLEWMGGQIYPGDGDTNYAQKFQGRVTITADESTSTAYMELSSLRSEDTAVYYCVRYYYGSSGYFDYWWGQGTMVTVSSGGGGSGGGGSGGGGSEIVLTQSPATLSLSPGERATLSCRASESVDNYGISFAWYQQKPGQAPRLLIYRASRATGIPARFSGSGSGTDFTLTISSLEPEDFAVYYCQQNNKDPPTFFGQGTKLEIKREPKSCDKTHTCPPCDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 703 aa, Artificial
  Car 005 translation region
MVLLVTSLLLCELPHPAFLLIPDTQVQLQQSGAELVKPGASVKISCKASGYAFSSYWMNWVKQRPGKGLEWIGQIYPGDGDTTYNGKFKGKATLTADKSSSTVYMQLNSLTSEDSAVYFCVRYYYGSSGYFDYWGQGTTLTVSSGGGGSGGGGSGGGGSDVQMIQTPDSLAVSLGQRATISCRASESVDNYGISFMHWYQQKPGQSPKLLIYRASNLESGIPARFSGSGSRTDFTLTINPVETDDVATYYCQQNNKDPPTFGGGTKLEIKEPKSCDKTHTCPPCDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 57 bp, Homo sapiens
atgggagccatcgggctcctgtggctcctgccgctgctgctttccacggcagctgtg

SEQ ID NO: 18 - DNA, 1149 bp, Homo sapiens
acagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacac

SEQ ID NO: 19 - protein, 8 aa, Artificial
  Histidine tag
HHHHHHHH

SEQ ID NO: 20 - protein, 12 aa, Artificial
  PA tag
GVAMPGAEDDVV

SEQ ID NO: 21 - DNA, 6 bp, Artificial
  Kozak sequence
gccacc

SEQ ID NO: 22 - DNA, 1278 bp, Artificial
  Kozak-ALK-His-PA
gccaccatgggtgccatcgggttgctgtggcttctccccctcttgctttctactgctgcagttactgccccgaaatcccgcaatctgtttgaacggaaccccaacaaggagctaaagcctggagagaattcaccgagacagactcccatattcgatccaacggtccattggctgttcaccacttgtggggctagtgggcctcatggccctacccaagcacagtgcaacaacgcctatcagaacagcaatctctctgtggaagtaggcagcgaagggccacttaaggggatccagatttggaaggtgcctgctacagacacctactccattagtggctatggtgcggcaggtgggaaaggaggcaagaacaccatgatgcgatcccatggcgtcagtgtactcggtatcttcaacctggagaaagatgacatgctgtacattctcgttggacaacagggtgaagacgcgtgcccatcgaccaaccagctgatccagaaagtgtgcataggcgagaataacgtcatcgaggaggagattcgggtgaatagatcggtgcatgagtgggcgggtgggggtggagggggcggaggggccacatacgttttcaagatgaaggatggcgtacccgttcccctgatcattgctgcaggtggcggtggaagggcctatggtgccaaaaccgacacctttcatcccgaaaggttagagaacaatagtagcgtgctaggcctgaatgggaattcaggcgctgcaggagggggaggcggatggaacgataatacgtccctcctgtgggccggcaaatccctgcaagagggcgctacaggaggacacagctgtcctcaggccatgaagaagtggggctgggaaacacgtggagggtttggggggggcggcggagggtgttcatctggaggcggcggcggtgggtatattggcggaaatgctgcatctaacaatgaccctgagatggatggcgaagatggtgtgtctttcatctcacccttgggcatactttacactcctgccctgaaagtgatggagggacacggagaggtgaatatcaagcactacttgaactgttcccactgcgaggtggacgaatgccacatggacccagaaagccacaaagtgatctgcttttgtgaccacggcacagtcttagccgaagatggagtcagctgcattgtgagccccacaccagagccgcaccaccatcatcaccatcaccaccatggggttgccatgccaggggctgaagacgatgtggtctgataa

SEQ ID NO: 23 - DNA, 1272 bp, Homo sapiens
atgggctgctggggacagctgctggtgtggttcggagccgcgggcgccattctctgctctagcccggggtcccaggagacttttctgcggtcctcgcccctgccgctggcaagtcccagcccccgggacccgaaagtcagcgccccgcctagtatcttggagccagcctccccgctgaattctccgggcaccgaggggtcttggctgttttctacctgcggggccagcggccggcatgggcccacacagacacaatgtgacggggcgtacgcggggaccagcgtggtggtgaccgtgggggccgccgggcagctgagaggcgtgcagctgtggcgcgtgccgggccctggccagtatctgatctcagcctacggagccgcgggcggcaaaggcgccaagaaccacctgtcgcgggcgcatggcgtcttcgtctcagcaatcttctccctcggtctcggggagtcgctgtacatcctggtggggcagcagggagaggacgcctgtcccggaggtagcccggagagccagctcgtctgcctcggggagtctcgagccgttgaagagcacgcggcgatggatgggagcgaaggggtcccggggtcgcggcgctgggcgggaggtggcgggggtggcgggggcgccacctacgttttccgggtgcgcgctggcgagctggaaccgttgctggtggcggccggaggcggcggtcgggcctacctgaggccgcgggaccgaggccggactcaggcctcccccgagaaactggagaaccgctcggaggcgcccgggagcggcgggagaggcggggcggcaggtggtgggggcggctggacgtcgcgggctccctctccgcaggccggccgctcactgcaggagggggcggagggcggccagggctgctccgaggcttgggcgacccttggctgggccgcggccggcggcttcgggggcggcggcggggcctgcactgcgggcggaggcggcggcggctacagggggggcgacgcttcagagactgacaacctctgggctgatggggaagatggagtatccttcatacaccccagcagcgagctcttcctgcagcctctggcagtcaccgagaaccacggagaggtagagatccgaaggcacctcaactgcagtcactgccctttgagagactgccaatggcaggcagagctccagctggctgaatgcctgtgcccagaaggcatggagctagctgtggataacgtcacctgcatggacctgcacaagcccccaggccct

SEQ ID NO: 24 - DNA, 1344 bp, Artificial
  Kozak-LTK-His-PA
gccaccatgggctgttggggtcagttgctggtgtggtttggagcagcaggggcaatcctctgcagctctccgggcagccaggaaacgttcctgaggtcctcccccttgcctttggccagcccttccccaagggatccgaaagtgtcagcgccaccctcgatcctcgagcctgcttcacccctgaatagccccggaactgaggggtcatggctattctctacttgcggcgcctctggtcggcatggccccacacagactcagtgcgatggtgcctatgcgggtaccagcgtagtggtgaccgttggggctgccggccagcttcggggggtacagctgtggagagtcccaggaccaggccagtacctgatatccgcatatggtgcagctggaggcaaaggcgcgaagaaccacttaagccgagcccatggcgtgttcgtctctgcgatcttttccctcggactcggggagtctttgtacattctggtagggcaacagggagaggatgcctgtcccggggggtcaccggaatcgcagcttgtgtgcttaggggaaagcagagccgtggaagagcacgcagccatggacggttctgaaggagtccctgggagtcgtcggtgggcaggtggaggtggaggaggcggaggggccacctacgtgttccgcgtgagagctggagaactcgaaccactcctggtggctgccgggggcggagggagggcatacttacgcccacgcgacagaggccggacacaagcctcaccagaaaagctcgagaacaggtccgaggcccccggaagtggcggcagaggaggagccgcaggcggtggcggtgggtggacaagtcgagcaccctcccctcaagctggaaggagtcttcaggaaggggctgagggcggacagggatgctcagaggcttgggccacccttggttgggctgccgcaggaggctttggggggggtggcggggcttgtactgccggcggaggtggcggcgggtatcgaggtggcgacgcttctgagacggacaatctgtgggccgatggcgaggacggcgttagcttcattcaccccagtagcgaactgtttctgcagccacttgctgtcacagagaaccatggcgaggttgagattcgtcggcacctaaactgctcccactgtcccctgcgcgattgtcagtggcaagccgaactgcaactggccgagtgtctgtgccctgaagggatggagctagcggtggacaatgtgacctgcatggacttgcacaagccacctggccctcaccatcatcaccaccatcaccatggggtcgctatgcctggcgccgaggatgatgtcgtttgataa

SEQ ID NO: 25 - protein, 8 aa, Artificial
  HRV3C protease cleavage sequence
LEVLFQGP

SEQ ID NO: 26 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 5 aa, Artificial
  G4S linker
GGGGS

SEQ ID NO: 28 - DNA, 792 bp, Artificial
  Kozak-HRV3C-Fc-His-PA
gccaccctggaagtgctgtttcagggacccgataaaacccatacctgtccaccatgtcctgctcccgaacttctgggaggaccaagtgtgttcctgtttccacctaaacccaaagacacgttgatgatatcccgtactccagaggtgacttgtgtggtggtggatgtgtctcatgaggatcctgaggtgaagttcaactggtatgtcgacggtgtggaagtccacaatgccaagaccaagcctagggaggagcagtacaactccacgtatcgggtagtctccgtactgacagtgctgcatcaggactggctcaatgggaaggagtacaaatgcaaggttagcaacaaggctcttcccgcgcccatcgaaaagaccatctctaaggccaaaggtcagcccagagaaccccaggtttatacactcccgccttcacgagatgagctgaccaagaaccaagtcagcctcacatgcttggtcaaaggcttttatccgagtgacattgcagtcgagtgggaatccaatggccaaccagagaacaactacaaaacaactccgcccgttctcgacagcgatgggagcttctttctgtactccaaactgaccgtagacaagtctcgctggcaacagggcaatgtgttcagttgcagcgtgatgcacgaagcccttcacaatcactacactcagaagtcactgtctctgagccctgggaaaggcggcggtggctcacatcaccatcaccaccaccatcacggagtggccatgcctggggcagaggatgacgttgtttgataa

SEQ ID NO: 29 - DNA, 447 bp, Artificial
  Kozak-FAM150A-HRV3C-His
gccaccatgcgcccactgaaaccaggtgctcctcttcctgccctgtttctgctggctctggcactgtctccccatggcgctcatggcagacctcgcggtcgtagaggagcaagggtgactgacaaagagcccaaaccgttgctcttccttccagccgcaggagctgggaggacaccctctggcagtaggtcagccgagattttccctcgggatagcaatctgaaggacaagttcatcaagcacttcacaggacccgtgacctttagcccagagtgctccaagcactttcaccgcctctactacaacacccgggaatgctccactcccgcctattataagcggtgtgccagactgctgacccgattggcggtttcaccgctctgtagccaaacgctcgaagtcctgtttcaggggcctcatcaccaccatcaccatcaccattgataa

SEQ ID NO: 30 - DNA, 516 bp, Artificial
  Kozak-FAM150B-HRV3C-His
gccaccatgagagggcctggacatcccctgcttctcggccttctcctggttctgggagctgctgggagaggcagaggaggggcagagcctcgggaaccagccgatggccaagcgcttctgaggctggtcgttgagctggtgcaggaactccggaaacaccacagtgccgagcacaaaggcctgcagctgcttggacgggactgtgcattgggtcgagccgaagctgcagggttgggtccctcaccggagcaaagggtggagatcgtgcctagagatctccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacagctggaagtcctctttcagggtcctcatcaccatcatcaccatcaccattgataa

SEQ ID NO: 31 - DNA, 35 bp, Artificial
  forward primer
gatccagcctccggactgccaccatgggtgccatc

SEQ ID NO: 32 - DNA, 35 bp, Artificial
  reverse primer
tgaaacagcacttccaggtgcggctctggtgtggg

SEQ ID NO: 33 - DNA, 35 bp, Artificial
  forward primer
gatccagcctccggactgccaccatgggctgttgg

SEQ ID NO: 34 - DNA, 35 bp, Artificial
  reverse primer
tgaaacagcacttccagagggccaggtggcttgtg

SEQ ID NO: 35 - DNA, 24 bp, Artificial
  forward primer
ctggaagtgctgtttcagggaccc

SEQ ID NO: 36 - DNA, 35 bp, Artificial
  reverse primer
atcaaactcattactaattatcaaacaacgtcatc

SEQ ID NO: 37 - protein, 662 aa, Artificial
  ALK 001P
MGAIGLLWLLPLLLSTAAVTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLEVLFQGPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSHHHHHHHHGVAMPGAEDDVV

SEQ ID NO: 38 - DNA, 1986 bp, Artificial
  ALK 001P coding sequence
atgggtgccatcgggttgctgtggcttctccccctcttgctttctactgctgcagttactgccccgaaatcccgcaatctgtttgaacggaaccccaacaaggagctaaagcctggagagaattcaccgagacagactcccatattcgatccaacggtccattggctgttcaccacttgtggggctagtgggcctcatggccctacccaagcacagtgcaacaacgcctatcagaacagcaatctctctgtggaagtaggcagcgaagggccacttaaggggatccagatttggaaggtgcctgctacagacacctactccattagtggctatggtgcggcaggtgggaaaggaggcaagaacaccatgatgcgatcccatggcgtcagtgtactcggtatcttcaacctggagaaagatgacatgctgtacattctcgttggacaacagggtgaagacgcgtgcccatcgaccaaccagctgatccagaaagtgtgcataggcgagaataacgtcatcgaggaggagattcgggtgaatagatcggtgcatgagtgggcgggtgggggtggagggggcggaggggccacatacgttttcaagatgaaggatggcgtacccgttcccctgatcattgctgcaggtggcggtggaagggcctatggtgccaaaaccgacacctttcatcccgaaaggttagagaacaatagtagcgtgctaggcctgaatgggaattcaggcgctgcaggagggggaggcggatggaacgataatacgtccctcctgtgggccggcaaatccctgcaagagggcgctacaggaggacacagctgtcctcaggccatgaagaagtggggctgggaaacacgtggagggtttggggggggcggcggagggtgttcatctggaggcggcggcggtgggtatattggcggaaatgctgcatctaacaatgaccctgagatggatggcgaagatggtgtgtctttcatctcacccttgggcatactttacactcctgccctgaaagtgatggagggacacggagaggtgaatatcaagcactacttgaactgttcccactgcgaggtggacgaatgccacatggacccagaaagccacaaagtgatctgcttttgtgaccacggcacagtcttagccgaagatggagtcagctgcattgtgagccccacaccagagccgcacctggaagtgctgtttcagggacccgataaaacccatacctgtccaccatgtcctgctcccgaacttctgggaggaccaagtgtgttcctgtttccacctaaacccaaagacacgttgatgatatcccgtactccagaggtgacttgtgtggtggtggatgtgtctcatgaggatcctgaggtgaagttcaactggtatgtcgacggtgtggaagtccacaatgccaagaccaagcctagggaggagcagtacaactccacgtatcgggtagtctccgtactgacagtgctgcatcaggactggctcaatgggaaggagtacaaatgcaaggttagcaacaaggctcttcccgcgcccatcgaaaagaccatctctaaggccaaaggtcagcccagagaaccccaggtttatacactcccgccttcacgagatgagctgaccaagaaccaagtcagcctcacatgcttggtcaaaggcttttatccgagtgacattgcagtcgagtgggaatccaatggccaaccagagaacaactacaaaacaactccgcccgttctcgacagcgatgggagcttctttctgtactccaaactgaccgtagacaagtctcgctggcaacagggcaatgtgttcagttgcagcgtgatgcacgaagcccttcacaatcactacactcagaagtcactgtctctgagccctgggaaaggcggcggtggctcacatcaccatcaccaccaccatcacggagtggccatgcctggggcagaggatgacgttgtt

SEQ ID NO: 39 - protein, 684 aa, Artificial
  LTK 001P
MGCWGQLLVWFGAAGAILCSSPGSQETFLRSSPLPLASPSPRDPKVSAPPSILEPASPLNSPGTEGSWLFSTCGASGRHGPTQTQCDGAYAGTSVVVTVGAAGQLRGVQLWRVPGPGQYLISAYGAAGGKGAKNHLSRAHGVFVSAIFSLGLGESLYILVGQQGEDACPGGSPESQLVCLGESRAVEEHAAMDGSEGVPGSRRWAGGGGGGGGATYVFRVRAGELEPLLVAAGGGGRAYLRPRDRGRTQASPEKLENRSEAPGSGGRGGAAGGGGGWTSRAPSPQAGRSLQEGAEGGQGCSEAWATLGWAAAGGFGGGGGACTAGGGGGGYRGGDASETDNLWADGEDGVSFIHPSSELFLQPLAVTENHGEVEIRRHLNCSHCPLRDCQWQAELQLAECLCPEGMELAVDNVTCMDLHKPPGPLEVLFQGPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSHHHHHHHHGVAMPGAEDDVV

SEQ ID NO: 40 - DNA, 2052 bp, Artificial
  LTK 001P coding sequence
atgggctgttggggtcagttgctggtgtggtttggagcagcaggggcaatcctctgcagctctccgggcagccaggaaacgttcctgaggtcctcccccttgcctttggccagcccttccccaagggatccgaaagtgtcagcgccaccctcgatcctcgagcctgcttcacccctgaatagccccggaactgaggggtcatggctattctctacttgcggcgcctctggtcggcatggccccacacagactcagtgcgatggtgcctatgcgggtaccagcgtagtggtgaccgttggggctgccggccagcttcggggggtacagctgtggagagtcccaggaccaggccagtacctgatatccgcatatggtgcagctggaggcaaaggcgcgaagaaccacttaagccgagcccatggcgtgttcgtctctgcgatcttttccctcggactcggggagtctttgtacattctggtagggcaacagggagaggatgcctgtcccggggggtcaccggaatcgcagcttgtgtgcttaggggaaagcagagccgtggaagagcacgcagccatggacggttctgaaggagtccctgggagtcgtcggtgggcaggtggaggtggaggaggcggaggggccacctacgtgttccgcgtgagagctggagaactcgaaccactcctggtggctgccgggggcggagggagggcatacttacgcccacgcgacagaggccggacacaagcctcaccagaaaagctcgagaacaggtccgaggcccccggaagtggcggcagaggaggagccgcaggcggtggcggtgggtggacaagtcgagcaccctcccctcaagctggaaggagtcttcaggaaggggctgagggcggacagggatgctcagaggcttgggccacccttggttgggctgccgcaggaggctttggggggggtggcggggcttgtactgccggcggaggtggcggcgggtatcgaggtggcgacgcttctgagacggacaatctgtgggccgatggcgaggacggcgttagcttcattcaccccagtagcgaactgtttctgcagccacttgctgtcacagagaaccatggcgaggttgagattcgtcggcacctaaactgctcccactgtcccctgcgcgattgtcagtggcaagccgaactgcaactggccgagtgtctgtgccctgaagggatggagctagcggtggacaatgtgacctgcatggacttgcacaagccacctggccctctggaagtgctgtttcagggacccgataaaacccatacctgtccaccatgtcctgctcccgaacttctgggaggaccaagtgtgttcctgtttccacctaaacccaaagacacgttgatgatatcccgtactccagaggtgacttgtgtggtggtggatgtgtctcatgaggatcctgaggtgaagttcaactggtatgtcgacggtgtggaagtccacaatgccaagaccaagcctagggaggagcagtacaactccacgtatcgggtagtctccgtactgacagtgctgcatcaggactggctcaatgggaaggagtacaaatgcaaggttagcaacaaggctcttcccgcgcccatcgaaaagaccatctctaaggccaaaggtcagcccagagaaccccaggtttatacactcccgccttcacgagatgagctgaccaagaaccaagtcagcctcacatgcttggtcaaaggcttttatccgagtgacattgcagtcgagtgggaatccaatggccaaccagagaacaactacaaaacaactccgcccgttctcgacagcgatgggagcttctttctgtactccaaactgaccgtagacaagtctcgctggcaacagggcaatgtgttcagttgcagcgtgatgcacgaagcccttcacaatcactacactcagaagtcactgtctctgagccctgggaaaggcggcggtggctcacatcaccatcaccaccaccatcacggagtggccatgcctggggcagaggatgacgttgtt

SEQ ID NO: 41 - DNA, 35 bp, Artificial
  forward primer for 1st PCR
gtgctcttccaaggaccagccgcaggagctgggag

SEQ ID NO: 42 - DNA, 39 bp, Artificial
  reverse primer for 1st and 2nd PCRs
ggtaccgcggccgcttcatcacgtttggctacagagcgg

SEQ ID NO: 43 - DNA, 43 bp, Artificial
  forward primer for 2nd PCR
cagagaacagattggaggtctggaagtgctcttccaaggacca

SEQ ID NO: 44 - DNA, 384 bp, Artificial
  synthetic polynucleotide for fusion protein
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggt

SEQ ID NO: 45 - protein, 128 aa, Artificial
  fusion protein
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGG

SEQ ID NO: 46 - protein, 216 aa, Artificial
  Ligand 001P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPAAGAGRTPSGSRSAEIFPRDSNLKDKFIKHFTGPVTFSPECSKHFHRLYYNTRECSTPAYYKRCARLLTRLAVSPLCSQT

SEQ ID NO: 47 - DNA, 648 bp, Artificial
  Ligand 001P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccagccgcaggagctgggaggacaccctctggcagtaggtcagccgagattttccctcgggatagcaatctgaaggacaagttcatcaagcacttcacaggacccgtgacctttagcccagagtgctccaagcactttcaccgcctctactacaacacccgggaatgctccactcccgcctattataagcggtgtgccagactgctgacccgattggcggtttcaccgctctgtagccaaacg

SEQ ID NO: 48 - DNA, 35 bp, Artificial
  forward primer for 1st PCR
gtgctcttccaaggaccagccgaagctgcagggtt

SEQ ID NO: 49 - DNA, 39 bp, Artificial
  reverse primer for 1st and 2nd PCRs
ggtaccgcggccgcttcatcactgtttgtcctccatgca

SEQ ID NO: 50 - protein, 218 aa, Artificial
  Ligand 002P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPAEAAGLGPSPEQRVEIVPRDLRMKDKFLKHLTGPLYFSPKCSKHFHRLYHNTRDCTIPAYYKRCARLLTRLAVSPVCMEDKQ

SEQ ID NO: 51 - DNA, 654 bp, Artificial
  Ligand 002P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccagccgaagctgcagggttgggtccctcaccggagcaaagggtggagatcgtgcctagagatctccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 52 - DNA, 33 bp, Artificial
  forward primer
gctgcaggggctggacgaactccctcaggcagt

SEQ ID NO: 53 - DNA, 34 bp, Artificial
  reverse primer
gcactgaacccctttcagtatggccaccagaaac

SEQ ID NO: 54 - DNA, 36 bp, Artificial
  forward primer
gcagaagcggctggtctgggtccctcaccagaacag

SEQ ID NO: 55 - DNA, 34 bp, Artificial
  reverse primer
gcactgaacccctttgaggatggctacgaggaac

SEQ ID NO: 56 - DNA, 1599 bp, Artificial
  CAR 007 translation region
atggagttcggactgtcctggctgtttctggtggccatactgaaaggggttcagtgcgctgcaggggctggacgaactccctcaggcagtcggtcagccgagattttccctcgggatagcaatctcaaggacaagttcatcaagcacttcaccggtcctgtgaccttctctcccgagtgtagcaagcactttcatcgcctgtattacaacacacgtgaatgctccacaccagcctactataagcggtgtgcgaggctcctcactcgcttggccgtaagtccgctttgcagccaaaccgaacccaagtcctgcgataaaacccacacatgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 57 - DNA, 1605 bp, Artificial
  CAR 008 translation region
atggagtttgggctttcctggttgttcctcgtagccatcctcaaaggggttcagtgcgcagaagcggctggtctgggtccctcaccagaacagcgagtcgagattgtgcctcgcgatctccgcatgaaggacaagttcctgaagcatctgacaggcccgctgtacttcagtcccaagtgtagcaaacactttcatcgcctgtatcacaacacacgggattgcaccatacccgcctattacaagaggtgtgcaaggctgctgactcgattggccgtttctcctgtgtgcatggaggacaaacaggagcccaagagctgcgacaagacgcacacctgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 58 - protein, 534 aa, Artificial
  Car 008 translation region
MEFGLSWLFLVAILKGVQCAEAAGLGPSPEQRVEIVPRDLRMKDKFLKHLTGPLYFSPKCSKHFHRLYHNTRDCTIPAYYKRCARLLTRLAVSPVCMEDKQEPKSCDKTHTCPPCDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - DNA, 41 bp, Artificial
  forward primer
gatccagcctccggactgccaccatggagacagacacactc

SEQ ID NO: 60 - DNA, 41 bp, Artificial
  reverse primer
atcaaactcattactaatcatcactgtttgtcctccatgca

SEQ ID NO: 61 - DNA, 35 bp, Artificial
  forward primer
gtgctcttccaaggaccaggggcagagcctcggga

SEQ ID NO: 62 - protein, 264 aa, Artificial
  Ligand 004P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPGAEPREPADGQALLRLVVELVQELRKHHSAEHKGLQLLGRDCALGRAEAAGLGPSPEQRVEIVPRDLRMKDKFLKHLTGPLYFSPKCSKHFHRLYHNTRDCTIPAYYKRCARLLTRLAVSPVCMEDKQ

SEQ ID NO: 63 - DNA, 792 bp, Artificial
  Ligand 004P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccaggggcagagcctcgggaaccagccgatggccaagcgcttctgaggctggtcgttgagctggtgcaggaactccggaaacaccacagtgccgagcacaaaggcctgcagctgcttggacgggactgtgcattgggtcgagccgaagctgcagggttgggtccctcaccggagcaaagggtggagatcgtgcctagagatctccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 64 - DNA, 35 bp, Artificial
  forward primer
ctcttccaaggaccagcattgggtcgagccgaagc

SEQ ID NO: 65 - DNA, 35 bp, Artificial
  forward primer
ctcttccaaggaccaggtcgagccgaagctgcagg

SEQ ID NO: 66 - DNA, 35 bp, Artificial
  forward primer
ctcttccaaggaccagctgcagggttgggtccctc

SEQ ID NO: 67 - DNA, 35 bp, Artificial
  forward primer
ctcttccaaggaccagggttgggtccctcaccgga

SEQ ID NO: 68 - DNA, 35 bp, Artificial
  forward primer
ctcttccaaggaccaggtccctcaccggagcaaag

SEQ ID NO: 69 - DNA, 35 bp, Artificial
  forward primer
ctcttccaaggaccatcaccggagcaaagggtgga

SEQ ID NO: 70 - DNA, 35 bp, Artificial
  forward primer
ctcttccaaggaccagagcaaagggtggagatcgt

SEQ ID NO: 71 - DNA, 35 bp, Artificial
  forward primer
ctcttccaaggaccaagggtggagatcgtgcctag

SEQ ID NO: 72 - DNA, 35 bp, Artificial
  forward primer
ctcttccaaggaccagagatcgtgcctagagatct

SEQ ID NO: 73 - DNA, 35 bp, Artificial
  forward primer
ctcttccaaggaccagtgcctagagatctccgcat

SEQ ID NO: 74 - DNA, 35 bp, Artificial
  forward primer
ctcttccaaggaccaagagatctccgcatgaagga

SEQ ID NO: 75 - DNA, 35 bp, Artificial
  forward primer
ctcttccaaggaccactccgcatgaaggacaagtt

SEQ ID NO: 76 - DNA, 35 bp, Artificial
  forward primer
ctcttccaaggaccaatgaaggacaagttcctgaa

SEQ ID NO: 77 - DNA, 35 bp, Artificial
  forward primer
ctcttccaaggaccagacaagttcctgaagcacct

SEQ ID NO: 78 - DNA, 35 bp, Artificial
  forward primer
ctcttccaaggaccattcctgaagcacctgacagg

SEQ ID NO: 79 - DNA, 35 bp, Artificial
  forward primer
ctcttccaaggaccaaagcacctgacaggcccact

SEQ ID NO: 80 - DNA, 35 bp, Artificial
  forward primer
ctcttccaaggaccactgacaggcccactgtactt

SEQ ID NO: 81 - DNA, 35 bp, Artificial
  forward primer
ctcttccaaggaccaggcccactgtacttcagccc

SEQ ID NO: 82 - DNA, 39 bp, Artificial
  reverse primer
caaactcattactaatcatcagtcctccatgcatacggg

SEQ ID NO: 83 - DNA, 39 bp, Artificial
  reverse primer
caaactcattactaatcatcacatgcatacgggagacac

SEQ ID NO: 84 - DNA, 24 bp, Artificial
  reverse primer
tggtccttggaagagcacttccag

SEQ ID NO: 85 - protein, 222 aa, Artificial
  Ligand 005P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPALGRAEAAGLGPSPEQRVEIVPRDLRMKDKFLKHLTGPLYFSPKCSKHFHRLYHNTRDCTIPAYYKRCARLLTRLAVSPVCMEDKQ

SEQ ID NO: 86 - DNA, 666 bp, Artificial
  Ligand 005P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccagcattgggtcgagccgaagctgcagggttgggtccctcaccggagcaaagggtggagatcgtgcctagagatctccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 87 - protein, 220 aa, Artificial
  Ligand 006P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPGRAEAAGLGPSPEQRVEIVPRDLRMKDKFLKHLTGPLYFSPKCSKHFHRLYHNTRDCTIPAYYKRCARLLTRLAVSPVCMEDKQ

SEQ ID NO: 88 - DNA, 660 bp, Artificial
  Ligand 006P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccaggtcgagccgaagctgcagggttgggtccctcaccggagcaaagggtggagatcgtgcctagagatctccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 89 - protein, 216 aa, Artificial
  Ligand 007P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPAAGLGPSPEQRVEIVPRDLRMKDKFLKHLTGPLYFSPKCSKHFHRLYHNTRDCTIPAYYKRCARLLTRLAVSPVCMEDKQ

SEQ ID NO: 90 - DNA, 648 bp, Artificial
  Ligand 007P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccagctgcagggttgggtccctcaccggagcaaagggtggagatcgtgcctagagatctccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 91 - protein, 214 aa, Artificial
  Ligand 008P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPGLGPSPEQRVEIVPRDLRMKDKFLKHLTGPLYFSPKCSKHFHRLYHNTRDCTIPAYYKRCARLLTRLAVSPVCMEDKQ

SEQ ID NO: 92 - DNA, 642 bp, Artificial
  Ligand 008P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccagggttgggtccctcaccggagcaaagggtggagatcgtgcctagagatctccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 93 - protein, 212 aa, Artificial
  Ligand 009P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPGPSPEQRVEIVPRDLRMKDKFLKHLTGPLYFSPKCSKHFHRLYHNTRDCTIPAYYKRCARLLTRLAVSPVCMEDKQ

SEQ ID NO: 94 - DNA, 636 bp, Artificial
  Ligand 009P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccaggtccctcaccggagcaaagggtggagatcgtgcctagagatctccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 95 - protein, 210 aa, Artificial
  Ligand 010P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPSPEQRVEIVPRDLRMKDKFLKHLTGPLYFSPKCSKHFHRLYHNTRDCTIPAYYKRCARLLTRLAVSPVCMEDKQ

SEQ ID NO: 96 - DNA, 630 bp, Artificial
  Ligand 010P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccatcaccggagcaaagggtggagatcgtgcctagagatctccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 97 - protein, 208 aa, Artificial
  Ligand 011P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPEQRVEIVPRDLRMKDKFLKHLTGPLYFSPKCSKHFHRLYHNTRDCTIPAYYKRCARLLTRLAVSPVCMEDKQ

SEQ ID NO: 98 - DNA, 624 bp, Artificial
  Ligand 011P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccagagcaaagggtggagatcgtgcctagagatctccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 99 - protein, 206 aa, Artificial
  Ligand 012P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPRVEIVPRDLRMKDKFLKHLTGPLYFSPKCSKHFHRLYHNTRDCTIPAYYKRCARLLTRLAVSPVCMEDKQ

SEQ ID NO: 100 - DNA, 618 bp, Artificial
  Ligand 012P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccaagggtggagatcgtgcctagagatctccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 101 - protein, 204 aa, Artificial
  Ligand 013P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPEIVPRDLRMKDKFLKHLTGPLYFSPKCSKHFHRLYHNTRDCTIPAYYKRCARLLTRLAVSPVCMEDKQ

SEQ ID NO: 102 - DNA, 612 bp, Artificial
  Ligand 013P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccagagatcgtgcctagagatctccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 103 - protein, 202 aa, Artificial
  Ligand 014P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPVPRDLRMKDKFLKHLTGPLYFSPKCSKHFHRLYHNTRDCTIPAYYKRCARLLTRLAVSPVCMEDKQ

SEQ ID NO: 104 - DNA, 606 bp, Artificial
  Ligand 014P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccagtgcctagagatctccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 105 - protein, 200 aa, Artificial
  Ligand 015P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPRDLRMKDKFLKHLTGPLYFSPKCSKHFHRLYHNTRDCTIPAYYKRCARLLTRLAVSPVCMEDKQ

SEQ ID NO: 106 - DNA, 600 bp, Artificial
  Ligand 015P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccaagagatctccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 107 - protein, 198 aa, Artificial
  Ligand 016P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPLRMKDKFLKHLTGPLYFSPKCSKHFHRLYHNTRDCTIPAYYKRCARLLTRLAVSPVCMEDKQ

SEQ ID NO: 108 - DNA, 594 bp, Artificial
  Ligand 016P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccactccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 109 - protein, 196 aa, Artificial
  Ligand 017P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPMKDKFLKHLTGPLYFSPKCSKHFHRLYHNTRDCTIPAYYKRCARLLTRLAVSPVCMEDKQ

SEQ ID NO: 110 - DNA, 588 bp, Artificial
  Ligand 017P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccaatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 111 - protein, 194 aa, Artificial
  Ligand 018P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPDKFLKHLTGPLYFSPKCSKHFHRLYHNTRDCTIPAYYKRCARLLTRLAVSPVCMEDKQ

SEQ ID NO: 112 - DNA, 582 bp, Artificial
  Ligand 018P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccagacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 113 - protein, 192 aa, Artificial
  Ligand 019P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPFLKHLTGPLYFSPKCSKHFHRLYHNTRDCTIPAYYKRCARLLTRLAVSPVCMEDKQ

SEQ ID NO: 114 - DNA, 576 bp, Artificial
  Ligand 019P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccattcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 115 - protein, 190 aa, Artificial
  Ligand 020P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPKHLTGPLYFSPKCSKHFHRLYHNTRDCTIPAYYKRCARLLTRLAVSPVCMEDKQ

SEQ ID NO: 116 - DNA, 570 bp, Artificial
  Ligand 020P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccaaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 117 - protein, 188 aa, Artificial
  Ligand 021P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPLTGPLYFSPKCSKHFHRLYHNTRDCTIPAYYKRCARLLTRLAVSPVCMEDKQ

SEQ ID NO: 118 - DNA, 564 bp, Artificial
  Ligand 021P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccactgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 119 - protein, 186 aa, Artificial
  Ligand 022P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPGPLYFSPKCSKHFHRLYHNTRDCTIPAYYKRCARLLTRLAVSPVCMEDKQ

SEQ ID NO: 120 - DNA, 558 bp, Artificial
  Ligand 022P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggacaaacag

SEQ ID NO: 121 - protein, 216 aa, Artificial
  Ligand 023P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPAEAAGLGPSPEQRVEIVPRDLRMKDKFLKHLTGPLYFSPKCSKHFHRLYHNTRDCTIPAYYKRCARLLTRLAVSPVCMED

SEQ ID NO: 122 - DNA, 648 bp, Artificial
  Ligand 023P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccagccgaagctgcagggttgggtccctcaccggagcaaagggtggagatcgtgcctagagatctccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatggaggac

SEQ ID NO: 123 - protein, 214 aa, Artificial
  Ligand 024P
METDTLLLWVLLLWVPGSTGDGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGLEVLFQGPAEAAGLGPSPEQRVEIVPRDLRMKDKFLKHLTGPLYFSPKCSKHFHRLYHNTRDCTIPAYYKRCARLLTRLAVSPVCM

SEQ ID NO: 124 - DNA, 642 bp, Artificial
  Ligand 024P coding sequence
atggagacagacacactcctgctatgggtactgctgctctgggttccaggttccactggtgacggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtctggaagtgctcttccaaggaccagccgaagctgcagggttgggtccctcaccggagcaaagggtggagatcgtgcctagagatctccgcatgaaggacaagttcctgaagcacctgacaggcccactgtacttcagcccaaaatgctccaagcactttcatcgcctgtatcacaacactcgcgattgcaccattcccgcctactataagcgttgtgccaggctgctgacgcgattggctgtgtctcccgtatgcatg

SEQ ID NO: 125 - protein, 114 aa, Homo sapiens
AAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCEL

SEQ ID NO: 126 - DNA, 372 bp, Artificial
  15bp+8a-41BB
ctgtctccgggtaaagccgcggcaaccaccactcctgctcccagaccacctactcccgctcccaccatagcctctcaacctctgagccttaggccagaagcctgtcgtcctgctgctggtggagccgttcacacaaggggattggactttgcctgcgacatctacatttgggcaccattggcaggcacatgtggcgtgcttctcctgtcactcgtcattaccctgtattgcaaacgagggcgcaagaagctgctgtacatcttcaaacagccctttatgcggccagtgcagacgacacaggaggaggatgggtgctcctgcagattcccggaagaggaggaaggtggctgtgagctgagagtgaagttcagc

SEQ ID NO: 127 - DNA, 33 bp, Artificial
  forward primer
ctgtctccgggtaaagccgcggcaaccaccact

SEQ ID NO: 128 - DNA, 33 bp, Artificial
  reverse primer
gctgaacttcactctcagctcacagccaccttc

SEQ ID NO: 129 - DNA, 28 bp, Artificial
  forward primer
agagtgaagttcagcaggagcgcagacg

SEQ ID NO: 130 - DNA, 28 bp, Artificial
  reverse primer
tttacccggagacagggagaggctcttc

SEQ ID NO: 131 - DNA, 1872 bp, Artificial
  CAR 009 translation region
atggagttcggactgtcctggctgtttctggtggccatactgaaaggggttcagtgcatgaggccactgaaaccgggagcacctcttcctgccctgtttctgctcgccttggcactgtctcctcatggcgctcatggcagaccacgcgggagaaggggtgctagagtcacggacaaagaacccaaacccctgctgtttcttccagctgcaggggctggacgaactccctcaggcagtcggtcagccgagattttccctcgggatagcaatctcaaggacaagttcatcaagcacttcaccggtcctgtgaccttctctcccgagtgtagcaagcactttcatcgcctgtattacaacacacgtgaatgctccacaccagcctactataagcggtgtgcgaggctcctcactcgcttggccgtaagtccgctttgcagccaaaccgaacccaagtcctgcgataaaacccacacatgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagccgcggcaaccaccactcctgctcccagaccacctactcccgctcccaccatagcctctcaacctctgagccttaggccagaagcctgtcgtcctgctgctggtggagccgttcacacaaggggattggactttgcctgcgacatctacatttgggcaccattggcaggcacatgtggcgtgcttctcctgtcactcgtcattaccctgtattgcaaacgagggcgcaagaagctgctgtacatcttcaaacagccctttatgcggccagtgcagacgacacaggaggaggatgggtgctcctgcagattcccggaagaggaggaaggtggctgtgagctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 132 - DNA, 1941 bp, Artificial
  CAR 010 translation region
atggagtttgggctttcctggttgttcctcgtagccatcctcaaaggggttcagtgcatgagagggccaggacatcctctgcttctgggcctgctgctggtgttgggcgctgctggcagaggtcgtggcggagcagagcctagagagccagccgatgggcaagcccttctgcggcttgtggtcgaactcgtgcaggaactgcggaaacaccattccgctgaacacaaaggcctgcaactgctcggaagagactgtgccttgggaagggcagaagcggctggtctgggtccctcaccagaacagcgagtcgagattgtgcctcgcgatctccgcatgaaggacaagttcctgaagcatctgacaggcccgctgtacttcagtcccaagtgtagcaaacactttcatcgcctgtatcacaacacacgggattgcaccatacccgcctattacaagaggtgtgcaaggctgctgactcgattggccgtttctcctgtgtgcatggaggacaaacaggagcccaagagctgcgacaagacgcacacctgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagccgcggcaaccaccactcctgctcccagaccacctactcccgctcccaccatagcctctcaacctctgagccttaggccagaagcctgtcgtcctgctgctggtggagccgttcacacaaggggattggactttgcctgcgacatctacatttgggcaccattggcaggcacatgtggcgtgcttctcctgtcactcgtcattaccctgtattgcaaacgagggcgcaagaagctgctgtacatcttcaaacagccctttatgcggccagtgcagacgacacaggaggaggatgggtgctcctgcagattcccggaagaggaggaaggtggctgtgagctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 133 - DNA, 1725 bp, Artificial
  CAR 011 translation region
atggagttcggactgtcctggctgtttctggtggccatactgaaaggggttcagtgcgctgcaggggctggacgaactccctcaggcagtcggtcagccgagattttccctcgggatagcaatctcaaggacaagttcatcaagcacttcaccggtcctgtgaccttctctcccgagtgtagcaagcactttcatcgcctgtattacaacacacgtgaatgctccacaccagcctactataagcggtgtgcgaggctcctcactcgcttggccgtaagtccgctttgcagccaaaccgaacccaagtcctgcgataaaacccacacatgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagccgcggcaaccaccactcctgctcccagaccacctactcccgctcccaccatagcctctcaacctctgagccttaggccagaagcctgtcgtcctgctgctggtggagccgttcacacaaggggattggactttgcctgcgacatctacatttgggcaccattggcaggcacatgtggcgtgcttctcctgtcactcgtcattaccctgtattgcaaacgagggcgcaagaagctgctgtacatcttcaaacagccctttatgcggccagtgcagacgacacaggaggaggatgggtgctcctgcagattcccggaagaggaggaaggtggctgtgagctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 134 - DNA, 1731 bp, Artificial
  CAR 012 translation region
atggagtttgggctttcctggttgttcctcgtagccatcctcaaaggggttcagtgcgcagaagcggctggtctgggtccctcaccagaacagcgagtcgagattgtgcctcgcgatctccgcatgaaggacaagttcctgaagcatctgacaggcccgctgtacttcagtcccaagtgtagcaaacactttcatcgcctgtatcacaacacacgggattgcaccatacccgcctattacaagaggtgtgcaaggctgctgactcgattggccgtttctcctgtgtgcatggaggacaaacaggagcccaagagctgcgacaagacgcacacctgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagccgcggcaaccaccactcctgctcccagaccacctactcccgctcccaccatagcctctcaacctctgagccttaggccagaagcctgtcgtcctgctgctggtggagccgttcacacaaggggattggactttgcctgcgacatctacatttgggcaccattggcaggcacatgtggcgtgcttctcctgtcactcgtcattaccctgtattgcaaacgagggcgcaagaagctgctgtacatcttcaaacagccctttatgcggccagtgcagacgacacaggaggaggatgggtgctcctgcagattcccggaagaggaggaaggtggctgtgagctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 135 - DNA, 2241 bp, Artificial
  CAR 013 translation region
atggtgctgctggttacctccctccttctctgcgagttgccgcatcccgcttttctcctgattcccgacacacaggtccagctccagcaatctggggcagaagtgaagaaacccgggtcaagtgtgaaagtctcctgtaaagcaagtggctatgcctttagcagctacatttcctgggttcgacaggcacctggccaaggattggaatggatgggtggacagatctatcctggggatggagacaccaattacgcccagaagtttcagggtcgcgtcacaataacggcagatgagtccactagcactgcctacatggagctgagctctcttcgctcagaggatacagccgtgtactattgcgtacggtactactacggctcttccggctatttcgactattggtggggacagggaaccatggtaaccgtgagttccggcggtggaggaagcggaggcggcgggtcaggaggcggtgggtctgagatcgttctgactcaatctccagctaccctgtctctgagcccaggcgaacgtgctaccttgtcatgcagagctagcgagtctgtggataactacggtatatcctttgcgtggtatcagcagaaacctgggcaagccccaaggctgctgatctatcgggccagtagggctactgggattccggccagatttagcgggagcggcagtggtacagacttcacgctgaccatctcaagccttgaacccgaagatttcgcggtgtattactgccagcagaacaataaggacccacctaccttcttcggccaaggcactaaactggagatcaagagagagcccaagtcctgtgacaagacacacacatgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagccgcggcaaccaccactcctgctcccagaccacctactcccgctcccaccatagcctctcaacctctgagccttaggccagaagcctgtcgtcctgctgctggtggagccgttcacacaaggggattggactttgcctgcgacatctacatttgggcaccattggcaggcacatgtggcgtgcttctcctgtcactcgtcattaccctgtattgcaaacgagggcgcaagaagctgctgtacatcttcaaacagccctttatgcggccagtgcagacgacacaggaggaggatgggtgctcctgcagattcccggaagaggaggaaggtggctgtgagctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 136 - DNA, 2238 bp, Artificial
  CAR 014 translation region
atggtcctgctcgtcacgtcactgcttctgtgcgagttgcctcatcctgccttcctgctgattcccgatacacaggtgcaacttcagcagtcaggcgcagaactggtcaaaccaggtgctagcgtgaagatctcctgcaaggctagtgggtacgcgttttcctcctactggatgaattgggtgaagcaacgaccaggcaaggggttggagtggataggtcagatttatccaggcgatggggatacaacatacaatgggaagttcaagggaaaggccacacttactgccgacaaatcctctagcaccgtgtacatgcagctgaactccctgacgtccgaagatagcgccgtttacttctgcgtgagatactactacggcagctcagggtatttcgactattggggtcaaggaaccactttgaccgtatcttctggcggaggcggcagtggtggaggtggctcaggaggcggagggtcagacgttcagatgatacagacacctgactctctggccgtttccctcgggcagagagccaccatcagttgtcgcgcttctgagtctgtggataactatgggatcagcttcatgcactggtatcagcagaaacctggccaaagtcccaaactcctcatctatcgggctagcaatctggaaagcggcattccggcacgttttagtggcagcggaagcaggaccgactttaccctgaccattaatccggtggaaactgacgatgtagcaacctactattgccagcagaacaacaaggatccacccacttttggaggtggaacaaagctggagatcaaagagcccaaaagctgtgacaaaacgcacacttgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagccgcggcaaccaccactcctgctcccagaccacctactcccgctcccaccatagcctctcaacctctgagccttaggccagaagcctgtcgtcctgctgctggtggagccgttcacacaaggggattggactttgcctgcgacatctacatttgggcaccattggcaggcacatgtggcgtgcttctcctgtcactcgtcattaccctgtattgcaaacgagggcgcaagaagctgctgtacatcttcaaacagccctttatgcggccagtgcagacgacacaggaggaggatgggtgctcctgcagattcccggaagaggaggaaggtggctgtgagctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 137 - protein, 623 aa, Artificial
  CAR 009 translation region
MEFGLSWLFLVAILKGVQCMRPLKPGAPLPALFLLALALSPHGAHGRPRGRRGARVTDKEPKPLLFLPAAGAGRTPSGSRSAEIFPRDSNLKDKFIKHFTGPVTFSPECSKHFHRLYYNTRECSTPAYYKRCARLLTRLAVSPLCSQTEPKSCDKTHTCPPCDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 646 aa, Artificial
  CAR 010 translation region
MEFGLSWLFLVAILKGVQCMRGPGHPLLLGLLLVLGAAGRGRGGAEPREPADGQALLRLVVELVQELRKHHSAEHKGLQLLGRDCALGRAEAAGLGPSPEQRVEIVPRDLRMKDKFLKHLTGPLYFSPKCSKHFHRLYHNTRDCTIPAYYKRCARLLTRLAVSPVCMEDKQEPKSCDKTHTCPPCDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 574 aa, Artificial
  CAR 011 translation region
MEFGLSWLFLVAILKGVQCAAGAGRTPSGSRSAEIFPRDSNLKDKFIKHFTGPVTFSPECSKHFHRLYYNTRECSTPAYYKRCARLLTRLAVSPLCSQTEPKSCDKTHTCPPCDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 576 aa, Artificial
  CAR 012 translation region
MEFGLSWLFLVAILKGVQCAEAAGLGPSPEQRVEIVPRDLRMKDKFLKHLTGPLYFSPKCSKHFHRLYHNTRDCTIPAYYKRCARLLTRLAVSPVCMEDKQEPKSCDKTHTCPPCDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 746 aa, Artificial; identical to SEQ ID NO: 15
  CAR 013 translation region

SEQ ID NO: 142 - protein, 745 aa, Artificial
  CAR 014 translation region
MVLLVTSLLLCELPHPAFLLIPDTQVQLQQSGAELVKPGASVKISCKASGYAFSSYWMNWVKQRPGKGLEWIGQIYPGDGDTTYNGKFKGKATLTADKSSSTVYMQLNSLTSEDSAVYFCVRYYYGSSGYFDYWGQGTTLTVSSGGGGSGGGGSGGGGSDVQMIQTPDSLAVSLGQRATISCRASESVDNYGISFMHWYQQKPGQSPKLLIYRASNLESGIPARFSGSGSRTDFTLTINPVETDDVATYYCQQNNKDPPTFGGGTKLEIKEPKSCDKTHTCPPCDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 19 aa, Homo sapiens
MEFGLSWLFLVAILKGVQC

SEQ ID NO: 144 - protein, 24 aa, Homo sapiens
MVLLVTSLLLCELPHPAFLLIPDT

SEQ ID NO: 145 - protein, 129 aa, Homo sapiens
MRPLKPGAPLPALFLLALALSPHGAHGRPRGRRGARVTDKEPKPLLFLPAAGAGRTPSGSRSAEIFPRDSNLKDKFIKHFTGPVTFSPECSKHFHRLYYNTRECSTPAYYKRCARLLTRLAVSPLCSQT

SEQ ID NO: 146 - protein, 80 aa, Homo sapiens
AAGAGRTPSGSRSAEIFPRDSNLKDKFIKHFTGPVTFSPECSKHFHRLYYNTRECSTPAYYKRCARLLTRLAVSPLCSQT

SEQ ID NO: 147 - protein, 152 aa, Homo sapiens
MRGPGHPLLLGLLLVLGAAGRGRGGAEPREPADGQALLRLVVELVQELRKHHSAEHKGLQLLGRDCALGRAEAAGLGPSPEQRVEIVPRDLRMKDKFLKHLTGPLYFSPKCSKHFHRLYHNTRDCTIPAYYKRCARLLTRLAVSPVCMEDKQ

SEQ ID NO: 148 - protein, 82 aa, Homo sapiens
AEAAGLGPSPEQRVEIVPRDLRMKDKFLKHLTGPLYFSPKCSKHFHRLYHNTRDCTIPAYYKRCARLLTRLAVSPVCMEDKQ

SEQ ID NO: 149 - protein, 29 aa, Artificial
  Hinge-Spacer-Hinge
EPKSCDKTHTCPPCDPAEPKSPDKTHTCP

SEQ ID NO: 150 - protein, 113 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 151 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 68 aa, Homo sapiens
FWVLVVVGGVLACYSLLVTVAFIIFWVRSKRSRLLHSDYMNMTPRRPGPTRKHYQPYAPPRDFAAYRS

SEQ ID NO: 153 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - protein, 102 aa, Homo sapiens
RPRGRRGARVTDKEPKPLLFLPAAGAGRTPSGSRSAEIFPRDSNLKDKFIKHFTGPVTFSPECSKHFHRLYYNTRECSTPAYYKRCARLLTRLAVSPLCSQT

SEQ ID NO: 155 - protein, 128 aa, Homo sapiens
GAEPREPADGQALLRLVVELVQELRKHHSAEHKGLQLLGRDCALGRAEAAGLGPSPEQRVEIVPRDLRMKDKFLKHLTGPLYFSPKCSKHFHRLYHNTRDCTIPAYYKRCARLLTRLAVSPVCMEDKQ

SEQ ID NO: 156 - DNA, 30 bp, Artificial
  forward primer for CAR 015 amplification
atgaaggacaagttcctgaagcatctgaca

SEQ ID NO: 157 - DNA, 30 bp, Artificial
  forward primer for CAR 016 amplification
gacaagttcctgaagcatctgacaggcccg

SEQ ID NO: 158 - DNA, 30 bp, Artificial
  forward primer for CAR 017 amplification
aagcatctgacaggcccgctgtacttcagt

SEQ ID NO: 159 - DNA, 30 bp, Artificial
  forward primer for CAR 018 amplification
ctgacaggcccgctgtacttcagtcccaag

SEQ ID NO: 160 - DNA, 30 bp, Artificial
  forward primer for CAR 019 amplification
ggcccgctgtacttcagtcccaagtgtagc

SEQ ID NO: 161 - DNA, 1539 bp, Artificial
  CAR 015 translation region
atggagtttgggctttcctggttgttcctcgtagccatcctcaaaggggttcagtgcatgaaggacaagttcctgaagcatctgacaggcccgctgtacttcagtcccaagtgtagcaaacactttcatcgcctgtatcacaacacacgggattgcaccatacccgcctattacaagaggtgtgcaaggctgctgactcgattggccgtttctcctgtgtgcatggaggacaaacaggagcccaagagctgcgacaagacgcacacctgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 162 - DNA, 1533 bp, Artificial
  CAR 016 translation region
atggagtttgggctttcctggttgttcctcgtagccatcctcaaaggggttcagtgcgacaagttcctgaagcatctgacaggcccgctgtacttcagtcccaagtgtagcaaacactttcatcgcctgtatcacaacacacgggattgcaccatacccgcctattacaagaggtgtgcaaggctgctgactcgattggccgtttctcctgtgtgcatggaggacaaacaggagcccaagagctgcgacaagacgcacacctgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 163 - DNA, 1522 bp, Artificial
  CAR 017 translation region
atggagtttgggctttcctggttgttcctcgtagccatcctcaaaggggttcagtgcaagcatctgacaggcccgctgtacttcagtcccaagtgtagcaaacactttcatcgcctgtatcacaacacacgggattgcaccatacccgcctattacaagaggtgtgcaaggctgctgactcgattggccgtttctcctgtgtgcatggaggacaaacaggagcccaagagctgcgacaagacgcacacctgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaag

SEQ ID NO: 164 - DNA, 1515 bp, Artificial
  CAR 018 translation region
atggagtttgggctttcctggttgttcctcgtagccatcctcaaaggggttcagtgcctgacaggcccgctgtacttcagtcccaagtgtagcaaacactttcatcgcctgtatcacaacacacgggattgcaccatacccgcctattacaagaggtgtgcaaggctgctgactcgattggccgtttctcctgtgtgcatggaggacaaacaggagcccaagagctgcgacaagacgcacacctgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 165 - DNA, 1509 bp, Artificial
  CAR 019 translation region
atggagtttgggctttcctggttgttcctcgtagccatcctcaaaggggttcagtgcggcccgctgtacttcagtcccaagtgtagcaaacactttcatcgcctgtatcacaacacacgggattgcaccatacccgcctattacaagaggtgtgcaaggctgctgactcgattggccgtttctcctgtgtgcatggaggacaaacaggagcccaagagctgcgacaagacgcacacctgtccaccgtgtgatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 166 - protein, 512 aa, Artificial
  CAR 015
MEFGLSWLFLVAILKGVQCMKDKFLKHLTGPLYFSPKCSKHFHRLYHNTRDCTIPAYYKRCARLLTRLAVSPVCMEDKQEPKSCDKTHTCPPCDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 167 - protein, 510 aa, Artificial
  CAR 016
MEFGLSWLFLVAILKGVQCDKFLKHLTGPLYFSPKCSKHFHRLYHNTRDCTIPAYYKRCARLLTRLAVSPVCMEDKQEPKSCDKTHTCPPCDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 168 - protein, 506 aa, Artificial
  CAR 017
MEFGLSWLFLVAILKGVQCKHLTGPLYFSPKCSKHFHRLYHNTRDCTIPAYYKRCARLLTRLAVSPVCMEDKQEPKSCDKTHTCPPCDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 169 - protein, 504 aa, Artificial
  CAR 018
MEFGLSWLFLVAILKGVQCLTGPLYFSPKCSKHFHRLYHNTRDCTIPAYYKRCARLLTRLAVSPVCMEDKQEPKSCDKTHTCPPCDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 170 - protein, 502 aa, Artificial
  CAR 019
MEFGLSWLFLVAILKGVQCGPLYFSPKCSKHFHRLYHNTRDCTIPAYYKRCARLLTRLAVSPVCMEDKQEPKSCDKTHTCPPCDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 171 - DNA, 39 bp, Artificial
  forward primer
gccgcggcaaccaccactcctgctcccagaccacctact

SEQ ID NO: 172 - DNA, 37 bp, Artificial
  reverse primer
tgggcatgtgtgagttttgtcaggagatttgggctcg

SEQ ID NO: 173 - DNA, 1071 bp, Artificial
  CAR 020 translation region
atggagtttgggctttcctggttgttcctcgtagccatcctcaaaggggttcagtgcgcagaagcggctggtctgggtccctcaccagaacagcgagtcgagattgtgcctcgcgatctccgcatgaaggacaagttcctgaagcatctgacaggcccgctgtacttcagtcccaagtgtagcaaacactttcatcgcctgtatcacaacacacgggattgcaccatacccgcctattacaagaggtgtgcaaggctgctgactcgattggccgtttctcctgtgtgcatggaggacaaacaggagcccaagagctgcgacaagacgcacacctgtccaccgtgtgatcccgccgagcccaaatctcctgacaaaactcacacatgcccagccgcggcaaccaccactcctgctcccagaccacctactcccgctcccaccatagcctctcaacctctgagccttaggccagaagcctgtcgtcctgctgctggtggagccgttcacacaaggggattggactttgcctgcgacatctacatttgggcaccattggcaggcacatgtggcgtgcttctcctgtcactcgtcattaccctgtattgcaaacgagggcgcaagaagctgctgtacatcttcaaacagccctttatgcggccagtgcagacgacacaggaggaggatgggtgctcctgcagattcccggaagaggaggaaggtggctgtgagctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 174 - protein, 356 aa, Artificial
  CAR 020
MEFGLSWLFLVAILKGVQCAEAAGLGPSPEQRVEIVPRDLRMKDKFLKHLTGPLYFSPKCSKHFHRLYHNTRDCTIPAYYKRCARLLTRLAVSPVCMEDKQEPKSCDKTHTCPPCDPAEPKSPDKTHTCPAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 175 - DNA, 2271 bp, Homo sapiens
atgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtg

SEQ ID NO: 176 - DNA, 2283 bp, Artificial
  HindIII-Kozak-ALK-DraIII
aagcttgccaccatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtg

SEQ ID NO: 177 - DNA, 2601 bp, Homo sapiens
cacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 178 - DNA, 2612 bp, Artificial
  DraIII-ALK-TGA-NotI
cacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgatgagcggccgc

SEQ ID NO: 179 - DNA, 4860 bp, Artificial
  pEHX-ALK translation region
atgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccc

SEQ ID NO: 180 - DNA, 39 bp, Artificial
  forward primer
taagcggccgccaccatgggctgttggggtcagttgctg

SEQ ID NO: 181 - DNA, 39 bp, Artificial
  reverse primer
ttagctagctccatcccttcagggcacagacactcggcc

SEQ ID NO: 182 - DNA, 1238 bp, Artificial
  NotI-Kozak-LTK-NheI
gcggccgccaccatgggctgttggggtcagttgctggtgtggtttggagcagcaggggcaatcctctgcagctctccgggcagccaggaaacgttcctgaggtcctcccccttgcctttggccagcccttccccaagggatccgaaagtgtcagcgccaccctcgatcctcgagcctgcttcacccctgaatagccccggaactgaggggtcatggctattctctacttgcggcgcctctggtcggcatggccccacacagactcagtgcgatggtgcctatgcgggtaccagcgtagtggtgaccgttggggctgccggccagcttcggggggtacagctgtggagagtcccaggaccaggccagtacctgatatccgcatatggtgcagctggaggcaaaggcgcgaagaaccacttaagccgagcccatggcgtgttcgtctctgcgatcttttccctcggactcggggagtctttgtacattctggtagggcaacagggagaggatgcctgtcccggggggtcaccggaatcgcagcttgtgtgcttaggggaaagcagagccgtggaagagcacgcagccatggacggttctgaaggagtccctgggagtcgtcggtgggcaggtggaggtggaggaggcggaggggccacctacgtgttccgcgtgagagctggagaactcgaaccactcctggtggctgccgggggcggagggagggcatacttacgcccacgcgacagaggccggacacaagcctcaccagaaaagctcgagaacaggtccgaggcccccggaagtggcggcagaggaggagccgcaggcggtggcggtgggtggacaagtcgagcaccctcccctcaagctggaaggagtcttcaggaaggggctgagggcggacagggatgctcagaggcttgggccacccttggttgggctgccgcaggaggctttggggggggtggcggggcttgtactgccggcggaggtggcggcgggtatcgaggtggcgacgcttctgagacggacaatctgtgggccgatggcgaggacggcgttagcttcattcaccccagtagcgaactgtttctgcagccacttgctgtcacagagaaccatggcgaggttgagattcgtcggcacctaaactgctcccactgtcccctgcgcgattgtcagtggcaagccgaactgcaactggccgagtgtctgtgccctgaagggatggagctagc

SEQ ID NO: 183 - DNA, 1384 bp, Artificial
  NheI-LTK-TAA-XbaI
gctagcggttgataacgtgacatgcatggatctgcacaaacctcccgggcctcttgtgctcatggtcgcagttgtcgctacttccacattgtccctgctgatggtttgcggggtcctcatcctggtcaagcagaagaagtggcagggtctgcaggagatgaggctgcccagccccgaacttgaactgtctaaactgaggaccagtgccattcggacagcacccaatccctactattgccaggttggcctcggccccgctcaaagctggccactccctcctggggtaacagaagtgagtccagccaatgtgaccctcctccgcgcacttggccatggagcctttggtgaggtgtatgaggggcttgtcattggcttgccaggagactcatctccactgcaagtggccatcaagaccctccctgagctgtgtagcccgcaggatgaactcgactttctgatggaagcgctgatcattagcaagtttcgccaccagaatatcgtgcgttgtgtggggctttccctgcgcgctactcctcggttgattttgctggagctcatgagtggcggagacatgaaatccttcctgagacattcccgaccgcatctggggcaacctagtcccctcgtaatgagggatctgctgcaactcgcacaggacatcgctcagggctgccactatctggaagagaaccacttcattcaccgagacattgcggccagaaattgcctgctgtcttgtgccgggccttctagggtggctaaaatcggggattttggcatggctcgggacatataccgggcctcatactaccgcagaggagatcgggcccttctcccggtgaagtggatgccacccgaagcctttctggaagggatcttcacctctaagacggattcctggagctttggcgtgcttctgtgggagatattctccctgggatatatgccttaccctggtagaacgaaccaggaggtgctggacttcgtcgtaggcggaggcaggatggatcctcccaggggatgtcctggaccagtttaccgaatcatgactcagtgctggcaacatgagcccgagcttagaccaagcttcgcatctatactggaacgcctgcagtattgcacccaggacccagacgtgttgaacagcctgctgcccatggaactgggccccactcccgaggaggagggcacaagtggactcgggaacagaagcctggagtgtttgcggccaccgcaaccccaggaacttagccctgagaagctgaaaagctggggtggctcaccattgggtccatggctgtcctcaggtctgaaaccactgaaatctcgcggactgcagccgcagaacttgtggaatcccacctaccgttcatgataatctaga

SEQ ID NO: 184 - DNA, 35 bp, Artificial
  forward primer
attgctagcggttgataacgtgacatgcatggatc

SEQ ID NO: 185 - DNA, 35 bp, Artificial
  reverse primer
taagcggccgcttatcatgaacggtaggtgggatt

SEQ ID NO: 186 - DNA, 1386 bp, Artificial
  NheI-LTK-TGATAA-NotI
gctagcggttgataacgtgacatgcatggatctgcacaaacctcccgggcctcttgtgctcatggtcgcagttgtcgctacttccacattgtccctgctgatggtttgcggggtcctcatcctggtcaagcagaagaagtggcagggtctgcaggagatgaggctgcccagccccgaacttgaactgtctaaactgaggaccagtgccattcggacagcacccaatccctactattgccaggttggcctcggccccgctcaaagctggccactccctcctggggtaacagaagtgagtccagccaatgtgaccctcctccgcgcacttggccatggagcctttggtgaggtgtatgaggggcttgtcattggcttgccaggagactcatctccactgcaagtggccatcaagaccctccctgagctgtgtagcccgcaggatgaactcgactttctgatggaagcgctgatcattagcaagtttcgccaccagaatatcgtgcgttgtgtggggctttccctgcgcgctactcctcggttgattttgctggagctcatgagtggcggagacatgaaatccttcctgagacattcccgaccgcatctggggcaacctagtcccctcgtaatgagggatctgctgcaactcgcacaggacatcgctcagggctgccactatctggaagagaaccacttcattcaccgagacattgcggccagaaattgcctgctgtcttgtgccgggccttctagggtggctaaaatcggggattttggcatggctcgggacatataccgggcctcatactaccgcagaggagatcgggcccttctcccggtgaagtggatgccacccgaagcctttctggaagggatcttcacctctaagacggattcctggagctttggcgtgcttctgtgggagatattctccctgggatatatgccttaccctggtagaacgaaccaggaggtgctggacttcgtcgtaggcggaggcaggatggatcctcccaggggatgtcctggaccagtttaccgaatcatgactcagtgctggcaacatgagcccgagcttagaccaagcttcgcatctatactggaacgcctgcagtattgcacccaggacccagacgtgttgaacagcctgctgcccatggaactgggccccactcccgaggaggagggcacaagtggactcgggaacagaagcctggagtgtttgcggccaccgcaaccccaggaacttagccctgagaagctgaaaagctggggtggctcaccattgggtccatggctgtcctcaggtctgaaaccactgaaatctcgcggactgcagccgcagaacttgtggaatcccacctaccgttcatgataagcggccgc

SEQ ID NO: 187 - DNA, 2592 bp, Artificial
  pEHX-LTK translation region
atgggctgttggggtcagttgctggtgtggtttggagcagcaggggcaatcctctgcagctctccgggcagccaggaaacgttcctgaggtcctcccccttgcctttggccagcccttccccaagggatccgaaagtgtcagcgccaccctcgatcctcgagcctgcttcacccctgaatagccccggaactgaggggtcatggctattctctacttgcggcgcctctggtcggcatggccccacacagactcagtgcgatggtgcctatgcgggtaccagcgtagtggtgaccgttggggctgccggccagcttcggggggtacagctgtggagagtcccaggaccaggccagtacctgatatccgcatatggtgcagctggaggcaaaggcgcgaagaaccacttaagccgagcccatggcgtgttcgtctctgcgatcttttccctcggactcggggagtctttgtacattctggtagggcaacagggagaggatgcctgtcccggggggtcaccggaatcgcagcttgtgtgcttaggggaaagcagagccgtggaagagcacgcagccatggacggttctgaaggagtccctgggagtcgtcggtgggcaggtggaggtggaggaggcggaggggccacctacgtgttccgcgtgagagctggagaactcgaaccactcctggtggctgccgggggcggagggagggcatacttacgcccacgcgacagaggccggacacaagcctcaccagaaaagctcgagaacaggtccgaggcccccggaagtggcggcagaggaggagccgcaggcggtggcggtgggtggacaagtcgagcaccctcccctcaagctggaaggagtcttcaggaaggggctgagggcggacagggatgctcagaggcttgggccacccttggttgggctgccgcaggaggctttggggggggtggcggggcttgtactgccggcggaggtggcggcgggtatcgaggtggcgacgcttctgagacggacaatctgtgggccgatggcgaggacggcgttagcttcattcaccccagtagcgaactgtttctgcagccacttgctgtcacagagaaccatggcgaggttgagattcgtcggcacctaaactgctcccactgtcccctgcgcgattgtcagtggcaagccgaactgcaactggccgagtgtctgtgccctgaagggatggagctagcggttgataacgtgacatgcatggatctgcacaaacctcccgggcctcttgtgctcatggtcgcagttgtcgctacttccacattgtccctgctgatggtttgcggggtcctcatcctggtcaagcagaagaagtggcagggtctgcaggagatgaggctgcccagccccgaacttgaactgtctaaactgaggaccagtgccattcggacagcacccaatccctactattgccaggttggcctcggccccgctcaaagctggccactccctcctggggtaacagaagtgagtccagccaatgtgaccctcctccgcgcacttggccatggagcctttggtgaggtgtatgaggggcttgtcattggcttgccaggagactcatctccactgcaagtggccatcaagaccctccctgagctgtgtagcccgcaggatgaactcgactttctgatggaagcgctgatcattagcaagtttcgccaccagaatatcgtgcgttgtgtggggctttccctgcgcgctactcctcggttgattttgctggagctcatgagtggcggagacatgaaatccttcctgagacattcccgaccgcatctggggcaacctagtcccctcgtaatgagggatctgctgcaactcgcacaggacatcgctcagggctgccactatctggaagagaaccacttcattcaccgagacattgcggccagaaattgcctgctgtcttgtgccgggccttctagggtggctaaaatcggggattttggcatggctcgggacatataccgggcctcatactaccgcagaggagatcgggcccttctcccggtgaagtggatgccacccgaagcctttctggaagggatcttcacctctaagacggattcctggagctttggcgtgcttctgtgggagatattctccctgggatatatgccttaccctggtagaacgaaccaggaggtgctggacttcgtcgtaggcggaggcaggatggatcctcccaggggatgtcctggaccagtttaccgaatcatgactcagtgctggcaacatgagcccgagcttagaccaagcttcgcatctatactggaacgcctgcagtattgcacccaggacccagacgtgttgaacagcctgctgcccatggaactgggccccactcccgaggaggagggcacaagtggactcgggaacagaagcctggagtgtttgcggccaccgcaaccccaggaacttagccctgagaagctgaaaagctggggtggctcaccattgggtccatggctgtcctcaggtctgaaaccactgaaatctcgcggactgcagccgcagaacttgtggaatcccacctaccgttca

SEQ ID NO: 188 - DNA, 23 bp, Artificial
  forward primer for ALK amplification
ccatcgtggcttttgacaatatc

SEQ ID NO: 189 - DNA, 23 bp, Artificial
  reverse primer for ALK amplification
ggtgctgtattctgcaggatctt

SEQ ID NO: 190 - DNA, 20 bp, Artificial
  forward primer for LTK amplification
gcggagggagggcatactta

SEQ ID NO: 191 - DNA, 20 bp, Artificial
  reverse primer for LTK amplification
tcggacctgttctcgagctt

SEQ ID NO: 192 - DNA, 930 bp, Artificial
  CAR 021 translation region
atggagtttgggctttcctggttgttcctcgtagccatcctcaaaggggttcagtgcgcagaagcggctggtctgggtccctcaccagaacagcgagtcgagattgtgcctcgcgatctccgcatgaaggacaagttcctgaagcatctgacaggcccgctgtacttcagtcccaagtgtagcaaacactttcatcgcctgtatcacaacacacgggattgcaccatacccgcctattacaagaggtgtgcaaggctgctgactcgattggccgtttctcctgtgtgcatggaggacaaacaggagcccaagagctgcgacaagacgcacacctgtccaccgtgtgatcccgccgagcccaaatctcctgacaaaactcacacatgcccattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 193 - DNA, 1437 bp, Artificial
  CAR 022 translation region
atggtcctgctcgtcacgtcactgcttctgtgcgagttgcctcatcctgccttcctgctgattcccgatacacaggtgcaacttcagcagtcaggcgcagaactggtcaaaccaggtgctagcgtgaagatctcctgcaaggctagtgggtacgcgttttcctcctactggatgaattgggtgaagcaacgaccaggcaaggggttggagtggataggtcagatttatccaggcgatggggatacaacatacaatgggaagttcaagggaaaggccacacttactgccgacaaatcctctagcaccgtgtacatgcagctgaactccctgacgtccgaagatagcgccgtttacttctgcgtgagatactactacggcagctcagggtatttcgactattggggtcaaggaaccactttgaccgtatcttctggcggaggcggcagtggtggaggtggctcaggaggcggagggtcagacgttcagatgatacagacacctgactctctggccgtttccctcgggcagagagccaccatcagttgtcgcgcttctgagtctgtggataactatgggatcagcttcatgcactggtatcagcagaaacctggccaaagtcccaaactcctcatctatcgggctagcaatctggaaagcggcattccggcacgttttagtggcagcggaagcaggaccgactttaccctgaccattaatccggtggaaactgacgatgtagcaacctactattgccagcagaacaacaaggatccacccacttttggaggtggaacaaagctggagatcaaagagcccaaaagctgtgacaaaacgcacacttgtccaccgtgtgatcccgccgagcccaaatctcctgacaaaactcacacatgcccattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 194 - protein, 310 aa, Artificial
  CAR 021 translation region
MEFGLSWLFLVAILKGVQCAEAAGLGPSPEQRVEIVPRDLRMKDKFLKHLTGPLYFSPKCSKHFHRLYHNTRDCTIPAYYKRCARLLTRLAVSPVCMEDKQEPKSCDKTHTCPPCDPAEPKSPDKTHTCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 195 - protein, 479 aa, Artificial
  CAR 022 translation region
MVLLVTSLLLCELPHPAFLLIPDTQVQLQQSGAELVKPGASVKISCKASGYAFSSYWMNWVKQRPGKGLEWIGQIYPGDGDTTYNGKFKGKATLTADKSSSTVYMQLNSLTSEDSAVYFCVRYYYGSSGYFDYWGQGTTLTVSSGGGGSGGGGSGGGGSDVQMIQTPDSLAVSLGQRATISCRASESVDNYGISFMHWYQQKPGQSPKLLIYRASNLESGIPARFSGSGSRTDFTLTINPVETDDVATYYCQQNNKDPPTFGGGTKLEIKEPKSCDKTHTCPPCDPAEPKSPDKTHTCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR